SEQUENCE LISTING (51 sequences)

SEQ ID NO: 1 - DNA, 24 bp, Artificial
  Forward primer for MMP-1 RNA in the human keratinocyte cell line HaCaT
agatgaaaggtggaccaacaattt

SEQ ID NO: 2 - DNA, 20 bp, Artificial
  Reverse primer for MMP-1 RNA in the human keratinocyte cell line HaCaT
ccaagagaatggccgagttc

SEQ ID NO: 3 - DNA, 32 bp, Artificial
  Probe for MMP-1 RNA in the human keratinocyte cell line HaCaT
agagagtacaacttacatcgtgttgcggctca

SEQ ID NO: 4 - DNA, 21 bp, Artificial
  Forward primer for MMP-10 RNA in the human keratinocyte cell line HaCaT.
aacagattttgtgggcaccag

SEQ ID NO: 5 - DNA, 21 bp, Artificial
  Reverse primer for MMP-10 RNA in the human keratinocyte cell line HaCaT.
ttcgcaagatgatgtgaatgg

SEQ ID NO: 6 - DNA, 27 bp, Artificial
  Probe for MMP-10 RNA in the human keratinocyte cell line HaCaT.
aggcagggggaggtccgtagagagact

SEQ ID NO: 7 - DNA, 15 bp, Artificial
  Forward primer for MMP-2 RNA in the human keratinocyte cell line HaCaT.
ccctcgcaagcccaa

SEQ ID NO: 8 - DNA, 22 bp, Artificial
  Reverse primer for MMP-2 RNA in the human keratinocyte cell line HaCaT.
cagatcaggtgtgtagccaatg

SEQ ID NO: 9 - DNA, 28 bp, Artificial
  Probe for MMP-2 RNA in the human keratinocyte cell line HaCaT.
tgggacaagaaccagatcacatacagga

SEQ ID NO: 10 - DNA, 21 bp, Artificial
  Forward primer for MMP-9 RNA in the human keratinocyte cell line HaCaT.
cctgagaaccaatctcaccga

SEQ ID NO: 11 - DNA, 21 bp, Artificial
  Reverse primer for MMP-9 RNA in the human keratinocyte cell line HaCaT.
gccacccgagtgtaaccatag

SEQ ID NO: 12 - DNA, 28 bp, Artificial
  Probe for MMP-9 RNA in the human keratinocyte cell line HaCaT.
aggcagctggcagaggaatacctgtacc

SEQ ID NO: 13 - DNA, 18 bp, Artificial
  Forward primer for TIMP-1 RNA in the human keratinocyte cell line HaCaT.
cacccacagacggccttc

SEQ ID NO: 14 - DNA, 20 bp, Artificial
  Reverse primer for TIMP-1 RNA in the human keratinocyte cell line HaCaT.
ctggtgtccccacgaacttg

SEQ ID NO: 15 - DNA, 24 bp, Artificial
  Probe for TIMP-1 RNA in the human keratinocyte cell line HaCaT.
ccctgatgacgaggtcggaattgc

SEQ ID NO: 16 - DNA, 21 bp, Artificial
  Forward primer for beta-carotene 15, 15′ - oxygenase RNA in the human keratinocyte cell line HaCaT.
aggaaagaacagctggagcct

SEQ ID NO: 17 - DNA, 18 bp, Artificial
  Reverse primer for beta-carotene 15, 15′ - oxygenase RNA in the human keratinocyte cell line HaCaT.
gttccctgcagccatgct

SEQ ID NO: 18 - DNA, 26 bp, Artificial
  Probe for beta-carotene 15, 15′ - oxygenase RNA in the human keratinocyte cell line HaCaT.
tgagggccaaagtgacaggcaagatt

SEQ ID NO: 19 - DNA, 23 bp, Artificial
  Forward primer for beta -carotene 9′, 10′ - oxygenase RNA in the human keratinocyte cell line HaCaT.
gctcaatggctctctacttcgaa

SEQ ID NO: 20 - DNA, 17 bp, Artificial
  Reverse primer for beta -carotene 9′, 10′ - oxygenase RNA in the human keratinocyte cell line HaCaT.
cagcgccatcccatcaa

SEQ ID NO: 21 - DNA, 31 bp, Artificial
  Probe for beta -carotene 9′, 10′ - oxygenase RNA in the human keratinocyte cell line HaCaT.
cgagtttgggaaggataagtacaatcattgg

SEQ ID NO: 22 - DNA, 22 bp, Artificial
  Forward primer for RAR-alpha RNA in the human keratinocyte cell line HaCaT.
gtcctcaggctaccactatggg

SEQ ID NO: 23 - DNA, 24 bp, Artificial
  Reverse primer for RAR-alpha RNA in the human keratinocyte cell line HaCaT.
tgtacaccatgttcttctggatgc

SEQ ID NO: 24 - DNA, 23 bp, Artificial
  Probe for RAR-alpha RNA in the human keratinocyte cell line HaCaT.
ctgcaagggcttcttccgccgca

SEQ ID NO: 25 - DNA, 24 bp, Artificial
  Forward primer for RAR-beta RNA in the human keratinocyte cell line HaCaT.
aaatcatcagggtaccactatggg

SEQ ID NO: 26 - DNA, 27 bp, Artificial
  Reverse primer for RAR-beta RNA in the human keratinocyte cell line HaCaT.
cggtgacaagtgtaaatcatattcttc

SEQ ID NO: 27 - DNA, 28 bp, Artificial
  Probe for RAR-beta RNA in the human keratinocyte cell line HaCaT.
ctgtgagggatgtaagggctttttccgc

SEQ ID NO: 28 - DNA, 19 bp, Artificial
  Forward primer for RAR-gamma RNA in the human keratinocyte cell line HaCaT.
gttcttctggatgcttcgg

SEQ ID NO: 29 - DNA, 22 bp, Artificial
  Reverse primer for RAR-gamma RNA in the human keratinocyte cell line HaCaT.
gtctacaagccatgcttcgtgt

SEQ ID NO: 30 - DNA, 23 bp, Artificial
  Probe for RAR-gamma RNA in the human keratinocyte cell line HaCaT.
aagaagcccttgcagccttcaca

SEQ ID NO: 31 - DNA, 19 bp, Artificial
  Forward primer for RXR-alpha RNA in the human keratinocyte cell line HaCaT.
aagcacatctgcgccatct

SEQ ID NO: 32 - DNA, 15 bp, Artificial
  Reverse primer for RXR-alpha RNA in the human keratinocyte cell line HaCaT.
tgcacccctcgcagc

SEQ ID NO: 33 - DNA, 25 bp, Artificial
  Probe for RXR-alpha RNA in the human keratinocyte cell line HaCaT.
accgctcctcaggcaagcactatgg

SEQ ID NO: 34 - DNA, 24 bp, Artificial
  Forward primer for RXR-beta RNA in the human keratinocyte cell line HaCaT
tctggatgatcaggtcatattgct

SEQ ID NO: 35 - DNA, 19 bp, Artificial
  Reverse primer for RXR-beta RNA in the human keratinocyte cell line HaCaT
tcggtgtgaaaaggaggca

SEQ ID NO: 36 - DNA, 24 bp, Artificial
  Probe for RXR-beta RNA in the human keratinocyte cell line HaCaT
cgggcaggctggaatgaactcctc

SEQ ID NO: 37 - DNA, 20 bp, Artificial
  Forward primer for RXR-gamma RNA in the human keratinocyte cell line HaCaT
gcctccaggaatcaacttgg

SEQ ID NO: 38 - DNA, 23 bp, Artificial
  Reverse primer for RXR-gamma RNA in the human keratinocyte cell line HaCaT
ttgatgtcctctgaactgctgac

SEQ ID NO: 39 - DNA, 28 bp, Artificial
  Probe for RXR-gamma RNA in the human keratinocyte cell line HaCaT
ccacccagctctcagctaaatgtggtca

SEQ ID NO: 40 - DNA, 20 bp, Artificial
  Forward primer for 18s RNA in the human keratinocyte cell line HaCaT.
cggctaccacatccaaggaa

SEQ ID NO: 41 - DNA, 18 bp, Artificial
  Reverse primer for 18s RNA in the human keratinocyte cell line HaCaT.
gctggaattaccgcggct

SEQ ID NO: 42 - DNA, 22 bp, Artificial
  Probe for 18s RNA in the human keratinocyte cell line HaCaT.
tgctggcaccagacttgccctc

SEQ ID NO: 43 - DNA, 24 bp, Artificial
  Forward primer for Integrin-alpha6 RNA in the human keratinocyte cell line HaCaT.
tttcccgtttctttcttgagttgt

SEQ ID NO: 44 - DNA, 23 bp, Artificial
  Reverse primer for Integrin-alpha6 RNA in the human keratinocyte cell line HaCaT.
tggaaaaggtaacttgtgagcca

SEQ ID NO: 45 - DNA, 35 bp, Artificial
  Probe for Integrin-alpha6 RNA in the human keratinocyte cell line HaCaT.
agactccgttaggttcagggagtttatctcctttt

SEQ ID NO: 46 - DNA, 18 bp, Artificial
  Forward primer for GADD34 RNA in the human keratinocyte cell line HaCaT.
cggaccctgagactcccc

SEQ ID NO: 47 - DNA, 23 bp, Artificial
  Reverse primer for GADD34 RNA in the human keratinocyte cell line HaCaT.
aaggccagaaaggtgcgcttctc

SEQ ID NO: 48 - DNA, 22 bp, Artificial
  Probe for GADD34 RNA in the human keratinocyte cell line HaCaT.
gaaatggacagtgaccttctcg

SEQ ID NO: 49 - DNA, 23 bp, Artificial
  Forward primer for gadd153 RNA in the human keratinocyte cell line HaCaT.
gcaagaggtcctgtcttcagatg

SEQ ID NO: 50 - DNA, 29 bp, Artificial
  Reverse primer for gadd153 RNA in the human keratinocyte cell line HaCaT.
cacctcctggaaatgaagaggaagaatca

SEQ ID NO: 51 - DNA, 24 bp, Artificial
  Probe for gadd153 RNA in the human keratinocyte cell line HaCaT.
gggtcaagagtggtgaagattttt

(19) United States
(12) Patent Application Publication
(10) Pub. No.: US 2016/0304957 A1
(43) Pub. Date: Oct. 20, 2016

(54) TREATMENT AND MODULATION OF GENE EXPRESSION AND SKIN AGING

(71) Applicant: DSM NUTRITIONAL PRODUCTS LTD., Basel (CH)

(72) Inventors: Karin WERTZ, Rheinfelden (DE); Petra Buchwald HUNZIKER, Magden (CH); Martin NEEB, Weil am Rhein (DE); Regina GORALCZYK, Grenzach-Wyhlen (DE); Willi HUNZIKER, Magden (CH); Georges RISS, Obermorschwiller (FR); Nicole SEIFERT, Rheinfelden (CH); Guido STEINER, Grenzach-Wyhlen (DE)

(21) Appl. No.: 15/085,907

(22) Filed: Mar. 30, 2016

Related U.S. Application Data
(63) Continuation of application No. 14/675,797, filed on Apr. 1, 2015, now abandoned.
Application No. 14/675,797 is a continuation of application No. 12/608,920, filed on Oct. 29, 2009, now abandoned.
Application No. 12/608,920 is a continuation-in-part of application No. 11/454,376, filed on Jun. 15, 2006, now abandoned.

(51) Int. Cl. C12Q 1/68; G01N 33/50

(52) U.S. Cl. CPC C12Q 1/6883; G01N 33/5044; C12Q 2600/148; C12Q 2600/158; G01N 2333/96494

(57) ABSTRACT

Methods and compositions for treating modulating and/or ameliorating non-light-induced, particularly non-UV-induced, skin aging in a human, for reducing the basal MMP-10 expression in unirradiated cells of an organism and/or reducing the basal MMP-1 RNA transcription and protein translation in unirradiated cells of an organism, and/or for modulating the effects of UVA-induced RNA transcription and polypeptide translation of a matrix metalloprotease, which include administering an effective amount of β-carotene, a precursor of β-carotene, a salt of β-carotene, or a combination of two or more thereof to an organism, particularly a mammal, more particularly a human, in need thereof.

### CROSS-REFERENCE TO RELATED APPLICATIONS

This application is a continuation of U.S. application Ser. No. 14/675,797, filed Apr. 1, 2015, which is a continuation of U.S. application Ser. No. 12/608,920, filed Oct. 29, 2009, (Abandoned), which is a continuation-in-part of U.S. application Ser. No. 11/454,376, filed Jun. 15, 2006, (Abandoned), the contents of which are incorporated herein by reference in their entireties.

### FIELD OF THE INVENTION

The present invention relates to methods and compositions for treating, preventing, and/or modulating aging of the skin. More particularly, the present invention relates to methods and compositions for modulating the expression of genes that effect or influence skin aging, including modulating matrix metalloprotease (MMP), particularly MMP-1, MMP-3, and MMP-10, transcription and protein expression in an organism, particularly a mammal, more particularly a human, by administering β-carotene, a precursor of β-carotene, a derivative of β-carotene, a salt of β-carotene, or a combination of two or more thereof to that organism. The invention also relates to methods of screening for compounds that modulate an effect of UV radiation on eukaryotic cells and/or promote cellular health.

### BACKGROUND OF THE INVENTION

Solar light has been implicated in the photoaging process via ultraviolet A (“UVA”) radiation (320 to 400 nm; UVA1 340-400 nm, UVA2 320-340 nm) [1, 2], in addition to ultraviolet B (“UVB”) radiation-mediated skin carcinogenesis [3, 4]. UVA induces reactive oxygen species, including singlet oxygen (“¹O₂”) [5-11], which, in turn, can mediate and/or regulate the expression level of a variety of genes, including genes involved in photoaging, via the transcription factor AP-2 [6], and like, UVB/UVA2, UVA1 activates stress-activated protein kinases [83]. ¹O₂-mediated gene induction has been shown for matrix metalloprotease-1 (“MMP-1”) [12, 13], heme oxygenase-1 [14], interleukin-1 (“IL-1”) and −6 (“IL-6”) [15], as well as ICAM-1 [16]. Inhibition or moderation of these molecular events may confer photoprotection on target cells.

Due to its excellent ¹O₂-quenching capacity [17-21, 66], β-carotene is a promising agent for the prevention of photoaging. Also, β-carotene accumulates in skin, with generally higher concentrations found in the epidermis than in the dermis [22]. In humans consuming a diet rich in fruits and vegetables, β-carotene is present in skin at concentrations of about 0.1-to-4 μM [22, 23], and may be further accumulated by supplementation[24]. A photoprotective effect of β-carotene is suggested by several observations. Various organisms, including bacteria, plants, and butterflies, employ β-carotene pigmentation as a means to increase their resistance to damage by irradiation[25]. In erythropoietic protoporphyria (EPP) patients, β-carotene supplementation at high doses (180 mg/d) alleviated the symptoms of photosensitization [26-29], which occurs due to accumulation of endogenous porphyrins. β-carotene quenches the ¹O₂ formed in the presence of these endogenous porphyrins in UVA-exposed skin [30]. β-carotene also has a mild sun screen effect (SPF2), if supplemented at a high concentration[26, 31-37]. β-carotene does not, however, act as an optical filter [38], since its absorption maximum lies outside the UVB/UVA range at around 460 nm.

In addition to its ¹O₂-quenching activity, β-carotene also represents the most important provitamin A, serving as a precursor for the signaling molecule retinoic acid (“RA”). It is thus conceivable that β-carotene could be locally metabolized to RA, and then act via retinoid pathways. Indeed, β-carotene metabolism to retinol has been shown in cultures of human skin fibroblasts, melanocytes and keratinocytes, which take up β-carotene and increase their intracellular retinol concomitantly [39, 40]. The efficacy of topical tretinoin (all-trans RA) in treating photoaging is well established [41-47]. RA acts by stimulating the proliferation of keratinocytes, while inhibiting terminal keratinocyte differentiation[48-51]. As a result, the thickness of the transit-amplifying (TA) keratinocyte layer in the epidermis is increased, leading to a smoother appearance of the skin. Moreover, RA can prevent UV induction of MMP-1 [45], and UV repression of dermal collagen expression[46].

Accordingly, it would be advantageous to provide methods and compositions for treating or preventing skin aging and reduction of basal matrix metalloprotease expression and MMP-1 RNA and protein expression in the cells of an organism susceptible to skin aging, as well as methods and compositions to promote cellular health and/or protect against cellular damage. In addition, it would be advantageous to provide a screening method that would allow for the identification of other compounds that produce similar effects on some or all of the genes that respond to treatment with β-carotene.

### SUMMARY OF THE INVENTION

One embodiment of the present invention is a method of treating or preventing non-light induced skin aging in an organism. This method includes administering an effective amount of β-carotene, a precursor of β-carotene, a derivative of β-carotene, a salt of β-carotene, or a combination of two or more thereof to an organism—particularly a mammal, more particularly a human—in need thereof.

Another embodiment of the present invention is a composition containing an amount of β-carotene, a precursor of β-carotene, a derivative of β-carotene, a salt of β-carotene, or a combination of two or more thereof effective to treat, ameliorate and/or prevent non-light-induced, particularly non-UV radiation-induced, skin aging—being effective to modulate the gene responsible for the non-UV radiation-induced skin aging.

A further embodiment of the present invention is a method of reducing the basal MMP-10 expression in unirradiated cells of an organism. This method includes administering an effective amount of β-carotene, a precursor of β-carotene, a derivative of β-carotene, a salt of β-carotene, or a combination of two or more thereof to the organism in need thereof.

An additional embodiment of the present invention is a method for the reduction of the basal MMP-1 RNA transcription and protein translation in unirradiated cells of an organism, including administering an effective amount of β-carotene, a precursor of β-carotene, a derivative of β-carotene, a salt of β-carotene, or a combination of two or more thereof to the organism in need thereof.

Another embodiment of the present invention is a method for modulating UVA-induced RNA transcription and polypeptide translation of a matrix metalloprotease (MMP). This method includes administering to an organism in need thereof an effective amount of a composition comprising β-carotene, a precursor of β-carotene, a derivative of β-carotene, a salt of β-carotene, or a combination of two or more thereof.

A further embodiment of the present invention is a method of treating or ameliorating UVA-induced photoaging. This method includes administering to an organism in need thereof an effective amount of a composition containing β-carotene, a precursor of β-carotene, a derivative of β-carotene, a salt of β-carotene, or a combination of two or more thereof, which is sufficient to ameliorate the UVA-induced photoaging.

Another embodiment of the present invention is a method of modulating the effects of UVA-induced gene expression on skin aging, comprising, prior to exposing the skin to UVA radiation, administering to an organism an amount of a composition containing a compound selected from the group consisting of β-carotene, a precursor of β-carotene, a derivative of β-carotene, a salt of β-carotene, and a combination of two or more thereof, which amount is effective to modulate the effects of UVA-induced gene expression on skin aging.

Another embodiment of the present invention is a composition for modulating the effects of UVA-induced gene expression on skin aging, comprising an amount of a compound selected from the group comprising or consisting of β-carotene, a precursor of β-carotene, a derivative of β-carotene, a salt of β-carotene, and a combination of two or more thereof, which amount is effective to modulate the effects of UVA-induced gene expression on skin aging.

An additional embodiment of the present invention is a composition for modulating the effect of UVA-induced RNA transcription and polypeptide translation of a matrix metalloprotease (MMP) containing an effective amount of β-carotene, a precursor of β-carotene, a derivative of β-carotene, a salt of β-carotene, or a combination of two or more thereof to modulate the transcription and translation of MMPs induced by exposure to UVA.

A further embodiment of the present invention is a method of enhancing UVA-induced tanning of the skin. This method includes administering to an organism, prior to exposure to UVA radiation, an amount of a composition containing a compound selected from the group comprising or consisting of β-carotene, a precursor of β-carotene, a derivative of β-carotene, a salt of β-carotene, and a combination of two or more thereof, which amount is effective to increase UVA-induced PAR-2 gene transcription.

Still a further embodiment of the present invention is a composition for enhancing UVA-induced tanning. This composition contains an amount of a compound selected from the group comprising or consisting of β-carotene, a precursor of β-carotene, a derivative of β-carotene, a salt of β-carotene, and a combination of two or more thereof, which amount is effective to increase UVA-induced PAR-2 gene transcription.

Another embodiment of the present invention is a method for promoting cell differentiation in UVA-irradiated cells of an organism, including administering to the organism in need thereof an amount of a compound selected from the group comprising or consisting of β-carotene, a precursor of β-carotene, a derivative of β-carotene, a salt of β-carotene, and a combination of two or more thereof, which amount is effective to downregulate transcription of a gene selected from the group comprising or consisting of BPAG1, integrin_{α6}, ILK, desmocollins, Cx45 and combinations thereof, or to up regulate transcription of a gene selected from the group comprising or consisting of Cx31, KLF4, GADD153, and a combination of two or more thereof.

A further embodiment of the present invention is a composition for promoting cell differentiation in UVA-irradiated cells of an organism. This composition contains an amount of a compound selected from the group comprising or consisting of β-carotene, a precursor of β-carotene, a derivative of β-carotene, a salt of β-carotene, and a combination of two or more thereof, which compound is effective to downregulate transcription of a gene selected from the group comprising or consisting of BPAG1, integrin_{α6}, ILK, desmocollins, Cx45, and combinations thereof, or to up regulate transcription of a gene selected from the group comprising or consisting of Cx31, KLF4, GADD153, and a combination of two or more thereof.

An additional embodiment of the present invention is a method for modulating stress-induced induction of a gene in an organism, which method includes administering to the organism an amount of a compound selected from the group comprising or consisting of β-carotene, a precursor of β-carotene, a derivative of β-carotene, a salt of β-carotene, and a combination of two or more thereof, which amount is effective to modulate the stress-induced induction of the gene.

Another embodiment of the present invention is a composition for modulating stress-induced induction of a gene in an organism. This composition contains a compound selected from the group comprising or consisting of β-carotene, a precursor of β-carotene, a derivative of β-carotene, a salt of β-carotene, and combinations thereof, wherein the compound is present in the composition in an amount effective to modulate the stress-induced induction of the gene.

Still an additional embodiment of the present invention is a method for screening for a compound that modulates an effect of UV irradiation on eukaryotic cells. This method includes the steps of a) contacting a sample of eukaryotic cells with the compound to be evaluated, b) irradiating the cells from (a) with UV radiation, c) comparing a gene expression profile of the cells contacted with the compound to a gene expression profile of control cells that were not contacted with the compound prior to the irradiation step in (b), and d) correlating a difference in the gene expression profile of the cells exposed to the compound and the control cells that were not exposed to the compound with an ability of the compound to modulate an effect of UV irradiation on the cells.

### BRIEF DESCRIPTION OF THE DRAWINGS

FIG. 1 shows dose and time dependency of β-carotene (βc) uptake by HaCaT cells. Cells were supplemented with 0.5, 1.5 or 3 μM β-carotene for various time periods. Media were changed daily during the first 3 days. Cellular β-carotene uptake was analyzed by HPLC. Values are means±standard deviation of an experiment with three replicates per time point and condition.

FIG. 2 shows depletion of cellular β-carotene stores by UVA irradiation. HaCaT cells were supplemented with 0.5, 1.5 or 3 μM β-carotene for 2 days prior to UVA (270 kJ/m²) irradiation. Cellular n-carotene content was analyzed by HPLC. Values are means±standard deviation from an experiment with three replicates.

FIGS. 3A and 3B show the time course of induction by ultraviolet A (“UVA”) irradiation of MMP-1 (3A) and MMP-10 (3B). HaCaT cells were pretreated with 1.5 μM β-carotene for 2 days prior to UVA (270 kJ/m²) irradiation. Gene expression at 1, 2.5, 5, and 18 hours after UVA irradiation was analyzed by quantitative reverse transcriptase-polymerase chain reaction (“QRT-PCR”). Gene regulations by UVA and β-carotene are expressed as fold induction relative to the placebo-treated, non-irradiated controls. The graphs show data from two independent experiments. Error bars indicate standard error.

FIGS. 4A-4F show the effect of β-carotene on UVA-induction of MMP-1 (4A), MMP-3 (4B), MMP-10 (4C), MMP-2 (4D), MMP-9 (4E), and TIMP-1 (4F). HaCaT cells were pretreated with 1.5 μM β-carotene for 2 days prior to UVA (270 kJ/m²) irradiation. Gene expression 5 hours after UVA irradiation was analyzed by QRT-PCR. Gene regulation by UVA and β-carotene is expressed as fold induction relative to the placebo-treated, non-irradiated controls. Values are geometric means±standard error from three independent experiments for MMP-2, MMP-9, and TIMP-1 and from eight independent experiments for MMP-1, MMP-3, and MMP-10.

FIGS. 5A-5C show the effect of β-carotene on D₂O-enhanced UVA induction of MMP-1 (5A), MMP-3 (5B), and MMP-10 (5C). HaCaT cells were pretreated for 2 days with 0.5, 1.5, or 3 μM β-carotene. The cells were irradiated with UVA (270 kJ/m²) either in D₂O-containing PBS or in H₂O-containing PBS, to analyze ¹O₂ (“singlet oxygen”) inducibility of genes. Gene expression five hours after UVA irradiation was analyzed by QRT-PCR. Values are geometric means±standard error from three independent experiments.

FIGS. 6A and 6B show the effect of β-carotene on UVA-induced secretion of MMP-1 (6A) and TIMP-1 (6B) by HaCaT cells. HaCaT cells were supplemented with 0.5, 1.5, or 3 μM β-carotene for 2 days prior to UVA (270 kJ/m²) irradiation. MMP-1 and TIMP-1 secretion 24 hours after irradiation was analyzed by ELISA. Each condition was represented by three replicates in the experiment. Values are means±standard error.

FIG. 7 shows the effect of β-carotene on transactivation of an RA-dependent reporter construct. HaCaT cells were transiently transfected with the reporter construct pGL3 (RARE)5 tk luc, containing five DR5-type retinoic acid response elements (“RAREs”). Luciferase activity was determined after 40 hours treatment with β-carotene. Values are means±standard error from two experiments with four replicates each.

FIG. 8 shows β-carotene non-significantly induced retinoic acid receptor β (“RARβ”) in a dose-dependent manner. HaCaT cells were pretreated for 2 days with 0.5, 1.5, or 3 μM β-carotene. The cells were irradiated with UVA (270 kJ/m²) either in D₂O-containing PBS or in H₂O-containing PBS, to analyze ¹O₂ inducibility of RARβ. RARβ expression 5 hours after UVA irradiation was analyzed by QRT-PCR. Gene regulation by UVA, D₂O, and β-carotene is expressed as fold induction relative to the placebo-treated, non-irradiated controls. Values are geometric means±standard error from three independent experiments.

FIGS. 9A-9C show β-carotene-induced inhibition of integrin_{α6} transcription in irradiated and unirradiated HaCaT cells (FIG. 9A) and enhancement of UVA-induced GADD34 (FIG. 9B) and GADD153 transcription (FIG. 9C). Cells were supplemented with β-carotene for 2 days prior to UVA irradiation (270 kJ/m²) either in normal PBS or D₂O-PBS. Gene expression 5 hours after irradiation was determined by quantitative real time polymerase chain reaction. (“QRT-PCR”). Values are geometric mean±standard error of three experiments.

FIG. 10 shows dose-dependent induction of caspase-3 activity in UVA-irradiated keratinocytes by β-carotene. Cells were supplemented with β-carotene for 2 days and prior to UVA irradiation (270 kJ/m²). Caspase-3 activity was determined at 5 hours after irradiation. Values are mean±standard error of an experiment with four replicates.

FIGS. 11A-11F show a model of molecular events, as deduced from the microarray data below; 11A and 11B show the effect of β-carotene treatment in unirradiated keratinocytes; 11C and 11D show the effect of UVA-irradiation in keratinocytes; and 11E and 11F show the effect of β-carotene treatment in UVA-irradiated keratinocytes. Genes labeled red were upregulated and genes labeled green were downregulated by the treatment. β-carotene treatment quenched the effect of UVA irradiation for genes labeled blue.

FIG. 12 shows the relationship of the modes of action of β-carotene to its influence on UVA-induced biological processes deduced from the experiments below.

### DETAILED DESCRIPTION OF THE INVENTION

One embodiment of the present invention is a method of treating or preventing non-light: induced skin aging in an organism. This method includes administering an effective amount of β-carotene, a precursor of β-carotene, a derivative of β-carotene, a salt of β-carotene, or a combination of two or more thereof to an organism in need thereof.

As used herein, the term “organism in need thereof” means an organism suffering from or susceptible to skin aging, for example, non-light-induced skin aging. Preferably, the organism is a mammal, more preferably, a human.

As used herein, the term “effective amount” means the amount of a composition or substance sufficient to produce the desired effect in the organism to which the composition or substance is administered. Preferably, an effective amount of β-carotene, a precursor of β-carotene, a derivative of β-carotene, a salt of β-carotene, or a combination of two or more thereof, is from about 1 milligram to about 30 milligrams per day. More preferably, an effective amount of β-carotene, a precursor of β-carotene, a derivative of β-carotene, a salt of β-carotene, or a combination of two or more thereof, is from about 5 milligrams to about 20 milligrams, even more preferably from about 10 milligrams to about 15 milligrams per day.

Another embodiment of the present invention is a composition containing an amount of β-carotene, a precursor of β-carotene, a derivative of β-carotene, a salt of β-carotene, or a combination of two or more thereof effective to treat or prevent non-light induced skin aging.

Effective dosage forms, modes of administration, and dosage amounts of β-carotene, a precursor of β-carotene, a derivative of β-carotene, a salt of β-carotene, or a combination of two or more thereof, or compositions containing β-carotene, a precursor of β-carotene, a derivative of β-carotene, a salt of β-carotene, or a combination of two or more thereof, according to the present invention, may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of animal, and like factors well known in the arts of medicine and veterinary medicine. In general, a suitable dose of β-carotene, a precursor of β-carotene, a derivative of β-carotene, a salt of β-carotene, or a combination of two or more thereof, according to the invention, will be that amount of the compound, which is the lowest dose effective to produce the desired effect. The effective dose of β-carotene, a precursor of β-carotene, a derivative of β-carotene, a salt of β-carotene, or a combination of two or more thereof, may be administered as a single dose or as two, three, four, five, six or more sub-doses, administered separately at appropriate intervals throughout the day.

The β-carotene, a precursor of β-carotene, a derivative of β-carotene, a salt of β-carotene, or a combination of two or more thereof, may be administered in any desired and effective manner: as a pharmaceutical compositions for oral ingestion, or for parenteral or other administration in any appropriate manner, such as intraperitoneal, subcutaneous, topical, intradermal, inhalation, intrapulmonary, rectal, vaginal, sublingual, intramuscular, intravenous, intraarterial, intrathecal, or intralymphatic. Preferably, the compound or composition is administered orally or topically. Further, the β-carotene, a precursor of β-carotene, a derivative of β-carotene, a salt of β-carotene, or a combination of two or more thereof, may be administered in conjunction with other treatments. The compound or composition may be encapsulated or otherwise protected against gastric or other secretions, if desired.

While it is possible for the β-carotene, a precursor of β-carotene, a derivative of β-carotene, a salt of β-carotene, or a combination of two or more thereof, of the invention to be administered alone, it is preferable to administer the β-carotene, a precursor of β-carotene, a derivative of β-carotene, a salt of β-carotene, or a combination of two or more thereof, as a pharmaceutical formulation (composition). The pharmaceutically-acceptable compositions of the invention comprise β-carotene, a precursor of β-carotene, a derivative of β-carotene, a salt of β-carotene, or a combination of two or more thereof, as an active ingredient in admixture with one or more pharmaceutically-acceptable carriers and, optionally, one or more other compounds, drugs, ingredients, and/or materials. Regardless of the route of administration selected, the β-carotene, a precursor of β-carotene, a derivative of β-carotene, a salt of n-carotene, or a combination of two or more thereof, of the present invention is formulated into pharmaceutically-acceptable dosage forms by conventional methods well known to those of skill in the art. See, e.g., Remington's Pharmaceutical Sciences (Mack Publishing Co., Easton, Pa.).

Pharmaceutical carriers are well known in the art (see, e.g., Remington's Pharmaceutical Sciences, op. cit., and The National Formulary (American Pharmaceutical Association, Washington, D.C.)), and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, and lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen β-carotene dosage form and method of administration may be determined using ordinary skill in the art.

The pharmaceutically-acceptable compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution: retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) emulsifying and suspending agents; (21) solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (22) propellants, such as chlorofluorohydrocarbons and volatile unsubstituted hydrocarbons, such as butane and propane; (23) antioxidants; (24) agents that render the formulation isotonic with the blood of the intended recipient, such as sugars and sodium chloride; (25) thickening agents; (26) coating materials, such as lecithin; and (27) sweetening, flavoring, coloring, perfuming and preservative agents. Each such ingredient or material, as with carriers, must be “acceptable” in the sense of being compatible with the other ingredients of the formulation and not injurious to the subject. Ingredients and materials suitable for a selected dosage form and intended route of administration are well known in the art, and acceptable ingredients and materials for a chosen β-carotene, a precursor of β-carotene, a derivative of β-carotene, a salt of β-carotene, or a combination of two or more thereof, dosage form and method of administration may be determined using ordinary skill in the art.

Pharmaceutical formulations suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution: retarding agents, absorption accelerators, wetting agents, absorbents, lubricants, and/or coloring agents. Solid compositions of a similar type maybe employed as fillers in soft- and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow: or controlled: release of the active ingredient(s) therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient may also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise β-carotene, a precursor of β-carotene, a derivative of β-carotene, a salt of β-carotene, or a combination of two or more thereof, in combination with one or more pharmaceutically-acceptable sterile isotonic aqueous or non-aqueous solutions, dispersions, suspensions or emulsions, or sterile powders that may be reconstituted into sterile injectable solutions or dispersions just prior to use, which may contain suitable antioxidants, buffers, solutes that render the formulation isotonic with the blood of the intended recipient, or suspending or thickening agents. Proper fluidity may be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug, it is desirable to slow its absorption from subcutaneous or intramuscular injection. This may be accomplished by the use of a liquid suspension of crystalline or amorphous material having poor water solubility.

The rate of absorption of the drug then depends upon its rate of dissolution, which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsule matrices of the active ingredient in biodegradable polymers. Depending on the ratio of the active ingredient to polymer and the nature of the particular polymer employed, the rate of active ingredient release can be controlled. Depot injectable formulations are also prepared by entrapping the drug in liposomes or microemulsions which are compatible with body tissue. The injectable materials may be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example, water for injection, immediately prior to use. Extemporaneous injection solutions and suspensions may be prepared from sterile powders, granules and tablets of the type described above.

In the present invention, the β-carotene, a precursor of β-carotene, a derivative of β-carotene, a salt of β-carotene, or a combination of two or more thereof, may be incorporated into various finished products, such as for example, a food, fortified food, functional food, food additive, clinical nutrition formulation, feed, fortified feed, functional feed, feed additive, beverage, dietary supplement, personal care product, nutraceutical, lotion, cream, spray, etc.

A further embodiment of the present invention is a method of reducing the basal MMP-10 expression in unirradiated cells of an organism. This method includes administering an effective amount of β-carotene, a precursor of β-carotene, a derivative of β-carotene, a salt of β-carotene, or a combination of two or more thereof to the organism in need thereof. In the present embodiment, the organisms, amounts of β-carotene, a precursor of β-carotene, a derivative of β-carotene, a salt of β-carotene, or a combination of two or more thereof, as well as delivery routes, and composition forms are as defined above.

An additional embodiment of the present invention is a method for the reduction of the basal MMP-1 RNA transcription and protein translation in unirradiated cells of an organism. This method includes administering an effective amount of β-carotene, a precursor of β-carotene, a derivative of β-carotene, a salt of β-carotene, or a combination of two or more thereof to the organism in need thereof. In the present embodiment, the organisms, amounts of β-carotene, a precursor of β-carotene, a derivative of β-carotene, a salt of β-carotene, or a combination of two or more thereof, as well as delivery routes, and composition forms are as defined above.

Another embodiment of the present invention is a method for ameliorating the effects of non-UV radiation-induced skin aging. This method includes administering to an organism in need thereof an amount of a compound selected from the group comprising or consisting of β-carotene, a precursor of β-carotene, a derivative of β-carotene, a salt of β-carotene, and combinations of two or more thereof, which amount is effective to modulate a gene responsible for the non-UV radiation-induced skin aging.

A further embodiment of the present invention is a composition for ameliorating the effects of non-UV radiation induced skin aging. This compound contains an amount of a compound selected from the group consisting of β-carotene, a precursor of β-carotene, a derivative of β-carotene, a salt of β-carotene, and combinations of two or more thereof, which amount is effective to modulate a gene responsible for the non-UV radiation induced skin aging. In the present invention, other forms of β-carotene are also contemplated.

Another embodiment of the present invention is a method for modulating UVA-induced RNA transcription and polypeptide translation of a matrix metalloprotease (MMP). This method includes administering to an organism in need thereof an effective amount of a composition comprising β-carotene, a precursor of β-carotene, a derivative of β-carotene, a salt of β-carotene, or a combination of two or more thereof.

As used herein, the term “modulation” means a reduction in the MMP RNA or protein levels compared to an organism to which the composition of β-carotene, a precursor of β-carotene, a derivative of β-carotene, a salt of β-carotene, or a combination of two or more thereof, is not administered.

Preferably, the MMP is selected from the group consisting of MMP-1, MMP-3, MMP-10, and combinations of two or more thereof. More preferably, the MMP is MMP-1 and MMP-10.

In the present embodiment, the organisms, amounts of β-carotene, a precursor of β-carotene, a derivative of β-carotene, a salt of β-carotene, or a combination of two or more thereof, as well as delivery routes, and composition forms are as defined above.

A further embodiment of the present invention is a method of treating or ameliorating UVA-induced photoaging. This method includes administering to an organism in need thereof an effective amount of a composition containing β-carotene, a precursor of β-carotene, a derivative of β-carotene, a salt of β-carotene, or a combination of two or more thereof, which is sufficient to ameliorate the UVA-induced photoaging.

Preferably, the effective amount of β-carotene, a precursor of β-carotene, a derivative of β-carotene, a salt of β-carotene, or a combination of two or more thereof is sufficient to reduce the level of MMP RNA transcripts and protein in the skin cells of the organism compared to the level in an organism to which the composition of β-carotene, a precursor of β-carotene, a derivative of β-carotene, a salt of β-carotene, or a combination of two or more thereof, is not administered. Preferably, the MMP is selected from the group consisting of MMP-1, MMP-3, MMP-10, and combinations of two or more thereof. More preferably, the MMP is MMP-1 and MMP-10.

In the present embodiment, the organisms, amounts of β-carotene, a precursor of β-carotene, a derivative of β-carotene, a salt of β-carotene, or a combination of two or more thereof, as well as delivery routes, and composition forms are as defined above.

Still another embodiment of the present invention is a method for screening for a compound that modulates an effect of UV irradiation on eukaryotic cells. This method includes the steps of: a) contacting a sample of eukaryotic cells with the compound to be evaluated, b) irradiating the cells from (a) with UV radiation, c) comparing a gene expression profile of the cells contacted with the compound to a gene expression profile of control cells that were not contacted with the compound prior to the irradiation step in (b), and d) correlating a difference in the gene expression profile of the cells exposed to the compound and the control cells that were not exposed to the compound with an ability of the compound to modulate an effect of UV irradiation on the cells.

In the present invention, the genetic profile analyzed is a transcriptome profile. A complete transcriptome refers to the complete set of mRNA transcripts produced by the genome at any one time. Unlike the genome, the transcriptome is dynamic and varies considerably in differing circumstances due to different patterns of gene expression. Transcriptomics, the study of the transcriptome, is a comprehensive means of identifying gene expression patterns. The transcriptome analyzed can include the complete known set of genes transcribed, i.e. the mRNA content or corresponding cDNA of a host cell or host organism. The cDNA can be a chain of nucleotides, an isolated polynucleotide, nucleotide, nucleic acid molecule, or any fragment or complement thereof that originated recombinantly or synthetically and be double-stranded or single-stranded, coding and/or noncoding, an exon or an intron of a genomic DNA molecule, or combined with carbohydrate, lipids, protein or inorganic elements or substances. The nucleotide chain can be at least 5, 10, 15, 30, 40, 50, 60, 70, 80, 90 or 100 nucleotides in length. The transcriptome can also include only a portion of the known set of genetic transcripts. For example, the transcriptome can include less than 98%, 95, 90, 85, 80, 70, 60, or 50% of the known transcripts in a host. The transcriptome can also be targeted to a specific set of genes.

In the present invention, the screening process can include screening using an array or a microarray to identify a genetic profile. In the present invention, the transcriptome or gene expression profile can be analyzed by using known processes such as hybridization in blot assays such as northern blots. In the present invention, the process can include PCR-based processes such as RT-PCR that can quantify expression of a particular set of genes.

The process can include analyzing the transcriptome or gene expression profile using a microarray or equivalent technique. In this process, the microarray can include at least a portion of the transcribed genome of the host cell, and typically includes binding partners to samples from genes of at least 50% of the transcribed genes of the organism. More typically, the microarray or equivalent technique includes binding partners for samples from at least 80%, 90%, 95%, 98%, 99% or 100% of the transcribed genes in the genome of the host cell. However, it is also possible that the microarray can include binding partners only to a selected subset of genes from the genome, including but not limited to putative genes that control or influence cellular health or protect against cellular damage. A microarray or equivalent technique can typically also include binding partners to a set of genes that are used as controls, such as housekeeper genes. A microarray or equivalent technique can also include genes clustered into groups such as genes coding for immediate early genes, oxidative defense genes, extracellular matrix genes, pro-inflammatory genes, VEGF-related ligand and receptor genes, IFNα/β genes, interleukin genes, proteinase-activated receptor genes, prostaglandin synthesis and signalling genes, EGF-related ligand and receptor genes, FGF-related ligand and receptor genes, TGF-β-related ligand and receptor genes, Wnt signalling genes, IGF/insulin signalling genes, Jagged/Delta signalling genes, MAPK pathway genes, differentiation marker genes, cell cycle genes, apoptosis genes, and combinations thereof.

A microarray is generally formed by linking a large number of discrete binding partners, which can include polynucleotides, aptamers, chemicals, antibodies or other proteins or peptides, to a solid support such as a microchip, glass slide, or the like, in a defined pattern. By contacting the microarray with a sample obtained from a cell of interest and detecting binding of the binding partners expressed in the cell that hybridize to sequences on the chip, the pattern formed by the hybridizing polynucleotides allows the identification of genes or clusters of genes that are expressed in the cell. Furthermore, where each member linked to the solid support is known, the identity of the hybridizing partners from the nucleic acid sample can be identified. One strength of microarray technology is that it allows the identification of differential gene expression simply by comparing patterns of hybridization.

Examples of high throughput screening processes include hybridization of host cell mRNA or substantially corresponding cDNA, to a hybridizable array(s) or microarray(s). The array or microarray can be one or more array(s) of nucleic acid or nucleic acid analog oligomers or polymers. In the present invention, the array(s) or microarray(s) may be independently or collectively a host-cell-genome-wide array(s) or microarray(s), containing a population of nucleic acid or nucleic acid analog oligomers or polymers whose nucleotide sequences are hybridizable to representative portions of all genes known to encode or predicted as encoding genes that control or influence cellular health or protect against cellular damage in the host cell strain. A genome-wide microarray includes sequences that bind to a representative portion of all of the known or predicted open reading frame (ORE) sequences, such as from mRNA or corresponding cDNA of the host.

The oligonucleotide sequences or analogs in the array typically hybridize to the mRNA or corresponding cDNA sequences from the host cell and typically comprise a nucleotide sequence complimentary to at least a portion of a host mRNA or cDNA sequence, or a sequence homologous to the host mRNA or cDNA sequence. Single DNA strands with complementary sequences can pair with each other and form double-stranded molecules.

Microarrays generally apply the hybridization principle in a highly parallel format. Instead of one identified, thousands of different potential identifieds can be arrayed on a miniature solid support. Instead of a unique labeled DNA probe, a complex mixture of labeled DNA molecules is used, prepared from the RNA of a particular cell type or tissue. The abundances of individual labeled DNA molecules in this complex probe typically reflect the expression levels of the corresponding genes. In a simplified process, when hybridized to the array, abundant sequences will generate strong signals and rare sequences will generate weak signals. The strength of the signal can represent the level of gene expression in the original sample.

In the present invention, a genome-wide array or microarray may be used. The array may represent more than 50% of the open reading frames in the genome of the host, or more than 55%, 60%, 65%, 70%, 75%, 80%, 85%, 90%, 91%, 92%, 93%, 94%, 95%, 96%, 97%, 98%, 99% or 100% of the known open reading frames in the genome. The array may also represent at least a portion of at least 50% of the sequences known to encode protein in the host cell. Alternatively, the array represents more than 50% of the genes or putative genes of the host cell, or more than 55%, 60%, 65%, 70%, 75%, 80%, 85%, 90%, 91%, 92%, 93%, 94%, 95%, 96%, 97%, 98%, 99% or 100% of the known genes or putative genes. In the present invention, more than one oligonucleotide or analog can be used for each gene or putative gene sequence or open reading frame. In the present invention, these multiple oligonucleotide or analogs represent different portions of a known gene or putative gene sequence. For each gene or putative gene sequence, from about 1 to about 10000 or from 1 to about 100 or from 1 to about 50, 45, 40, 35, 30, 25, 20, 15, 10 or less oligonucleotides or analogs can be present on the array.

A microarray or a complete genome-wide array or microarray may be prepared according to any process known in the art, based on knowledge of the sequence(s) of the host cell genome, or the proposed coding sequences in the genome, or based on the knowledge of expressed mRNA sequences in the host cell or host organism.

For different types of host cells, the same type of microarray can be applied. The types of microarrays include complementary DNA (cDNA) microarrays (Schena, M. et al. (1995) Quantitative monitoring of gene expression patterns with a complementary DNA microarray. Science 270:467-70) and oligonucleotide microarrays (Lockhart, et al. (1996) Expression monitoring by hybridization to high-density oligonucleotide arrays. Nat Biotechnol 14:1675-80). For cDNA microarray, the DNA fragment of a partial or entire open reading frame is printed on the slides. The hybridization characteristics can be different throughout the slide because different portions of the molecules can be printed in different locations. For the oligonucleotide arrays, 20-80-mer oligos can be synthesized either in situ (on-chip) or by conventional synthesis followed by on-chip immobilization, however in general all probes are designed to be similar with regard to hybridization temperature and binding affinity (Butte, A. (2002) The use and analysis of microarray data. Nat Rev Drug Discov 1:951-60).

In analyzing the transcriptome profile or gene expression, the nucleic acid or nucleic acid analog oligomers or polymers can be RNA, DNA, or an analog of RNA or DNA. Such nucleic acid analogs are known in the art and include, e.g.: peptide nucleic acids (PNA); arabinose nucleic acids; altritol nucleic acids; bridged nucleic acids (BNA), e.g., 2′-O,4′-C-ethylene bridged nucleic acids, and 2′-O,4′-C-methylene bridged nucleic acids; cyclohexenyl nucleic acids; 2′,5′-linked nucleotide-based nucleic acids; morpholino nucleic acids (nucleobase-substituted morpholino units connected, e.g., by phosphorodiamidate linkages); backbone-substituted nucleic acid analogs, e.g., 2′-substituted nucleic acids, wherein at least one of the 2′ carbon atoms of an oligo- or poly-saccharide-type nucleic acid or analog is independently substituted with, e.g., any one of a halo, thio, amino, aliphatic, oxyaliphatic, thioaliphatic, or aminoaliphatic group (wherein aliphatic is typically C₁-C₁₀ aliphatic).

Oligonucleotides or oligonucleotide analogs in the array can be of uniform size and, for example, can be about 10 to about 1000 nucleotides, about 20 to about 1000, 20 to about 500, 20 to about 100, about 20, about 25, about 30, about 40, about 50, about 60, about 70, about 80, about 90 or about 100 nucleotides long.

The array of oligonucleotide probes can be a high density array comprising greater than about 100, or greater than about 1,000 or more different oligonucleotide probes. Such high density arrays can comprise a probe density of greater than about 60, more generally greater than about 100, most generally greater than about 600, often greater than about 1000, more often greater than about 5,000, most often greater than about 10,000, typically greater than about 40,000 more typically greater than about 100,000, and in certain instances is greater than about 400,000 different oligonucleotide probes per cm² (where different oligonucleotides refers to oligonucleotides having different sequences). The oligonucleotide probes range from about 5 to about 500, or about 5 to 50, or from about 5 to about 45 nucleotides, or from about 10 to about 40 nucleotides and most typically from about 15 to about 40 nucleotides in length. Particular arrays contain probes ranging from about 20 to about 25 oligonucleotides in length. The array may comprise more than 10, or more than 50, or more than 100, and typically more than 1000 oligonucleotide probes specific for each identified gene. In the present invention, the array may comprise at least 10 different oligonucleotide probes for each gene. Alternatively, the array may have 20 or fewer oligonucleotides complementary each gene. Although a planar array surface is typical, the array may be fabricated on a surface of virtually any shape or even on multiple surfaces.

The array may further comprise mismatch control probes. Where such mismatch controls are present, the quantifying step may comprise calculating the difference in hybridization signal intensity between each of the oligonucleotide probes and its corresponding mismatch control probe. The quantifying may further comprise calculating the average difference in hybridization signal intensity between each of the oligonucleotide probes and its corresponding mismatch control probe for each gene.

In some assay formats, the oligonucleotide probe can be tethered, i.e., by covalent attachment, to a solid support. Oligonucleotide arrays can be chemically synthesized by parallel immobilized polymer synthesis processes or by light directed polymer synthesis processes, for example on poly-L-lysine substrates such as slides. Chemically synthesized arrays are advantageous in that probe preparation does not require cloning, a nucleic acid amplification step, or enzymatic synthesis. The array includes test probes which are oligonucleotide probes each of which has a sequence that is complementary to a subsequence of one of the genes (or the mRNA or the corresponding antisense cRNA) whose expression is to be detected. In addition, the array can contain normalization controls, mismatch controls and expression level controls as described herein.

An array may be designed to include one hybridizing oligonucleotide per known gene in a genome. The oligonucleotides or equivalent binding partners can be 5′-amino modified to support covalent binding to epoxy-coated slides. The oligonucleotides can be designed to reduce cross-hybridization, for example by reducing sequence identity to less than 25% between oligonucleotides. Generally, melting temperature of oligonucleotides is analyzed before design of the array to ensure consistent GC content and Tₘ, and secondary structure of oligonucleotide binding partners is optimized. For transcriptome or gene expression profiling, secondary structure is typically minimized. An array may have each oligonucleotide printed at at least two different locations on the slide to increase accuracy. Control oligonucleotides can also be designed based on sequences from different species than the host cell or organism to show background binding.

The samples in the genetic profile can be analyzed individually or grouped into clusters. The clusters can typically be grouped by similarity in gene expression. In the present invention, the clusters may be grouped individually as genes that are regulated to a similar extent in a host cell. The clusters may also include groups of genes that are regulated to a similar extent in a recombinant host cell, for example genes that are up-regulated or down-regulated to a similar extent compared to a host cell or a modified or an unmodified cell. The clusters can also include groups related by gene or protein structure, function or, in the case of a transcriptome or gene expression array, by placement or grouping of binding partners to genes in the genome of the host.

Groups of binding partners or groups of genes or proteins analyzed can include, but are not limited to: immediate early genes, oxidative defense genes, extracellular matrix genes, pro-inflammatory genes, VEGF-related ligand and receptor genes, IFNα/β genes, interleukin genes, proteinase-activated receptor genes, prostaglandin synthesis and signalling genes, EGF-related ligand and receptor genes, FGF-related ligand and receptor genes, TGF-β-related ligand and receptor genes, Wnt signalling genes, IGF/insulin signalling genes, Jagged/Delta signalling genes, MAPK pathway genes, Differentiation marker genes, cell cycle genes, apoptosis genes, and combinations thereof. Genes in these groups include, but are not limited to: genes coding for putative or known C-FOS, FRA-1, JUN-D, JUN-B, MAF-F, C-MYC, OSR-1, GEM, DKK-1, GADD34, GADD153, IEX-1, TSSC3/IPL, TDAG51, MMP-1, MMP-3, MMP-10, serpinB1, lekti, PAR-2, VEGF, IL-6, HB-EGF, SMADs, EGFR HER3, Wnt5A, FGFR2, cyclin E, ODC, ID1-3, ID-4, RB, KI67, thymidylate synthase, DNA ligase III, CENP-E, centromere and spindle protein genes, COL4, COL7, Cx31, BPAG1, integrin_{α6}, KLF4, and ILK.

As used herein, the term “organism in need thereof” means an organism suffering from or susceptible to skin aging, for example, non-light induced skin aging. Preferably, the organism is a mammal, more preferably, a human.

As used herein, the terms “effective amount” “amount . . . effective” or like terms mean the amount of a composition or substance sufficient to produce modulation of the expression of the gene or genes of interest in the organism to which the composition or substance is administered. Preferably, an effective amount of β-carotene or other compound according to the present invention is from about 1 milligram to about 30 milligrams per day. More preferably, an effective amount of β-carotene is from about 5 milligrams to about 20 milligrams, even more preferably from about 10 milligrams to about 15 milligrams per day. In the present invention, “modulation,” “modulate,” or like terms mean an up regulation, down regulation or quenching of gene expression caused by β-carotene or other compound/composition of interest.

Non-limiting examples of genes responsible for non-UV radiation skin aging are genes selected from the group comprising or consisting of a member of the stress signal family of genes, a member of the ECM degradation family of genes, a member of the immune modulation family of genes, a member of the inflammation-causing family of genes, a member of the cellular differentiation family of genes, and combinations thereof. Preferably, the cellular differentiation family of genes is selected from the group comprising or consisting of growth factor signalling genes, cell cycle regulation genes, differentiation genes, apoptosis genes, and combinations thereof. Preferably, the growth factor signalling genes are selected from the group comprising or consisting of EGFR, HER-3, FGF3, FRZ-6, NOTCH3, BMP2a, Wnt5a, and combinations thereof and the cell cycle regulation genes are selected from the group comprising or consisting of G1, RB, p21, ID-2, DNA ligase III, DNA-PK G2/M, BUB1, and combinations thereof.

Preferably, the immune modulation and inflammation family of genes are selected from the group comprising or consisting of VEGF, IL-18, COX-2, and combinations thereof. Preferably, the ECM degradation family of genes is selected from the group comprising or consisting of MMP-1, MMP-10, and combinations thereof. Preferably, the stress signal family of genes is selected from the group comprising or consisting of JUN-B, FRA-2, NRF-2, GEM, EGRa, TSSC3/IPL, and combinations thereof.

An additional embodiment of the present invention is a composition for modulating the effect of UVA-induced RNA transcription and polypeptide translation of a matrix metalloprotease containing an effective amount of β-carotene, a precursor of β-carotene, a derivative of β-carotene, a salt of β-carotene, or a combination thereof to modulate the transcription and translation of MMPs induced by exposure to UVA.

In the present embodiment, the organisms, amounts of β-carotene, a precursor of β-carotene, a derivative of β-carotene, a salt of β-carotene, or a combination of two or more thereof, as well as delivery routes, and composition forms are as defined above.

Still an additional embodiment of the present invention is a method of modulating the effects of UVA-induced gene expression on skin aging. This method includes, prior to exposure to UV-A radiation, administering to an organism an amount of a composition containing a compound selected from the group comprising or consisting of β-carotene, a precursor of β-carotene, a derivative of β-carotene, a salt of β-carotene, and combinations thereof, which amount is effective to modulate the effects of UV-A-induced gene expression on skin aging.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

Another embodiment of the present invention is a composition for modulating the effects of UVA-induced gene expression on skin aging. This composition includes an amount of a compound selected from the group comprising or consisting of β-carotene, a precursor of β-carotene, a derivative of β-carotene, a salt of β-carotene, and combinations thereof, which amount is effective to modulate the effects of UVA-induced gene expression on skin aging.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

A further embodiment of the present invention is a method of enhancing UVA-induced tanning of the skin. This method includes administering to an organism, prior to exposure to UVA radiation, an amount of a composition containing a compound selected from the group comprising or consisting of β-carotene, a precursor of β-carotene, a derivative of β-carotene, a salt of β-carotene, and combinations thereof, which amount is effective to increase UVA-induced PAR-2 gene transcription.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

An additional embodiment of the present invention is a composition for enhancing UVA-induced tanning. This composition contains an amount of a compound selected from the group comprising or consisting of β-carotene, a precursor of β-carotene, a derivative of β-carotene, a salt of β-carotene, and combinations thereof, which amount is effective to increase UVA-induced PAR-2 gene transcription.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

Another embodiment of the present invention is a method for promoting cell differentiation in UVA-irradiated cells of an organism. This method includes administering to the organism in need thereof an amount of a compound selected from the group comprising or consisting of β-carotene, a precursor of β-carotene, a derivative of β-carotene, a salt of β-carotene, and combinations thereof, which amount is effective to downregulate transcription of a gene selected from the group comprising or consisting of BPAG1, integrin_{α6}, ILK, desmocollins, Cx45 and combinations thereof or upregulate transcription of a gene selected from the group comprising or consisting of Cx31, KLF4, GADD153, and combinations thereof.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

A further embodiment of the present invention is a composition for promoting cell differentiation in UVA irradiated cells of an organism. This composition contains an amount of a compound selected from the group comprising or consisting of β-carotene, a precursor of β-carotene, a derivative of β-carotene, a salt of β-carotene, and combinations thereof, which compound is effective to downregulate transcription of a gene selected from the group comprising or consisting of BPAG1, integrin_{α6}, ILK, desmocollins, Cx45, and combinations thereof or to up regulate transcription of a gene selected from the group comprising or consisting of Cx31, KLF4, GADD153, and combinations thereof.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

An additional embodiment of the present invention is a method for modulating stress-induced induction of a gene in an organism. This method includes administering to the organism an amount of a compound selected from the group comprising or consisting of β-carotene, a precursor of β-carotene, a derivative of β-carotene, a salt of β-carotene, and combinations thereof, which amount is effective to modulate the stress-induced induction of the gene.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

Another embodiment of the present invention is a composition for modulating stress-induced induction of a gene in an organism. This composition contains a compound selected from the group comprising or consisting of β-carotene, a precursor of β-carotene, a derivative of β-carotene, a salt of β-carotene, and combinations thereof, wherein the compound is present in the composition in an amount effective to modulate the stress-induced induction of the gene.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

The following examples are provided to further illustrate the compositions and methods of the present invention. These examples are illustrative only and are not intended to limit the scope of the invention in any way.

### EXAMPLES

### Summary

UVA exposure causes skin photoaging by ¹O₂-mediated induction of, e.g., matrix metalloproteases. We assessed whether pretreatment with β-carotene, a ¹O₂ quencher and retinoic acid (RA) precursor, interferes with UVA-induced gene regulation. HaCaT keratinocytes were precultured with β-carotene at physiological concentrations (0.5, 1.5 and 3.0 μM) prior to UVA exposure from a Hönle solar simulator (270 kJ/m²). HaCaT cells accumulated β-carotene in a time and dose-dependent manner. UVA irradiation massively reduced the cellular β-carotene contents. β-carotene suppressed UVA-induction of MMP-1, MMP-3, and MMP-10, three major matrix metalloproteases involved in photoaging. We show that not only MMP-1, but also MMP-10, regulation involves ¹O₂-dependent mechanisms. β-carotene dose-dependently quenched ¹O₂-mediated induction of MMP-1 and MMP-10. Thus, as in chemical solvent systems, β-carotene quenches ¹O₂ also in living cells. Vitamin E did not cooperate with β-carotene to further inhibit MMP induction. HaCaT cells produced weak retinoid activity from β-carotene, as demonstrated by mild upregulation of RAR and activation of an RARE-dependent reporter gene. β-Carotene did not regulate the genes encoding other RARs, retinoid receptors (“RXR”), or the two β-carotene cleavage enzymes. These results demonstrate that β-carotene is photoprotective, and that this effect is mediated by ¹O₂ quenching.

### Materials and Methods

### Cell Culture

HaCaT cells were obtained from Prof. Fusenig, German Cancer Research Centre, Heidelberg [64]. To gather cells more representative for the upper epidermal layer, we cloned the original cells by endpoint dilution. A subclone was selected, which subclone had a polygonal epithelial morphology and exhibited the highest differentiation capacity. The clone expressed cytokeratins 1 and 10 starting from day 3 in culture, as detected by Western blotting. Cytokeratins were detected using anti-cytokeratin clones AE1/AE3 (Boehringer Mannheim, Germany) and anti-cytokeratin 1,10 antibody (Biogenesis Ltd., Poole, UK), respectively. Moreover, this clone expressed cytokeratins 1 and 10, as well as involucrin on the RNA level at 3 days post seeding (Wertz et al., unpublished observations). The doubling time of the clone was 16 hours and identical to the parent cell line. Cells were propagated in FAD medium (DMEM/HAM's F12 3:1, Invitrogen); 5% NuSerum IV culture supplement and Miton™ 1:1000 (both Becton Dickinson, Bedford, Mass., USA). On day 0 of the experiment, cells were seeded at 2×10⁵ cells per 60 mm dish. Cells were counted using a Coulter Multisizer (IG Instrumenten Gesellschaft, Zurich, Switzerland). The accuracy of cell counting was approximately 99%. On days 1 and 2, the media were replaced with fresh β-carotene-containing FAD medium without phenol red; 2% NuSerum; penicillin/streptomycin.

### Preparation of β-Carotene-Containing Medium

β-Carotene stock solutions and β-carotene-containing media were prepared under reduced light conditions. All-E-β-carotene was synthesized by DSM Nutritional Products (Kaiseraugst, Switzerland). β-carotene was dissolved in tetrahydrofurane (THF containing 0.025% butyl hydroxytoluol; Fluka Chemie A G, Buchs, Switzerland). Immediately before preparing the β-carotene stock solution, THF was purified over a basic aluminum oxide grade 1 (Camag, Muttenz, Switzerland) column. The n-carotene stock solution was prepared fresh for each experiment, and stored under argon at −20° C. until use. To prepare β-carotene-containing medium, the β-carotene stock solution was first diluted 1:1 with ethanol. This β-carotene/solvent mixture was then added to the cell culture medium to give a final concentration of 0.5, 1.5, or 3 μM β-carotene. β-carotene-containing medium was prepared fresh for the daily medium changes. The solvent concentration in the medium was kept constant at 0.5% for all treatment conditions. In previous experiments, it had been verified that the solvent at this concentration is not toxic for HaCaT cells.

### Preparation of Vitamin E-Containing Medium

Vitamin E (RRR-α-tocopherol; DSM Nutritional Products, Kaiseraugst, Switzerland) stock solutions and vitamin E-containing media were prepared as described for β-carotene-containing solutions, except that vitamin E was dissolved in ethanol. In experiments addressing the vitamin E effect, vitamin E was used in a final concentration of 50 μM. Again, the solvent concentration was kept constant at 0.03% ethanol for all conditions.

### UVA/Simulated Solar Radiation (SSR) Exposure

On day 3 of the experiment, cells were washed six times with Ca/Mg-free PBS containing 2% BSA, and then irradiated with light with a Hönle sun lamp Sol 500 (Dr. Hönle, Plannegg, Germany) at a dose of 270 kJ/m² in Ca/Mg-free PBS (2 hour exposure time at 3.77 mW/m²). The experimental schedule was chosen because the cells had optimal UVA sensitivity after 48 hours in culture. At that time, the cultures had a confluency of about 95%. Confluent cultures were much less sensitive to irradiation.

The spectrum of the Honle lamp simulates natural sun light with the majority of the spectrum between 320 and 750 nm. The minor UVB component was further reduced to 0.7 W/m² by placing a glass plate adjacent to the metal-halogenide light source. Thus, the light contained mainly the UVA 1 and UVA2 and visible light fraction. The dose calculation was based on the UVA measurement. Since an effect of visible light on human skin requires higher doses [65] of 1260 kJ/m², we refer to the major active light spectrum as UVA. Pilot experiments with increasing doses of UVA ranging from 50 to 300 kJ/m² showed a maximum response of HaCaT cells with 270 to 300 kJ/m² with respect to MMP-1 induction.

After irradiation, the cells were supplied with fresh β-carotene-containing serum-free medium and kept in the incubator at 37° C., 5% CO₂ until harvest of the samples. In experiments in which the half-life of ¹O₂ was prolonged by D₂O to enhance its effect [18], PBS was prepared in D₂O, instead of in H₂O, as it was done for the standard conditions described above. Cells were washed twice in D₂O-PBS prior to irradiation in D₂O-PBS. After irradiation, cells were maintained with fresh β-carotene-containing serum-free medium. Sham controls were treated in an identical manner, by placing them under the solar simulator but shielded from light.

### HPLC Analysis of Cell Culture Media and Cell Cultures

All extraction and analytical procedures were carried out in brown glass, and under reduced light conditions. Acetonitrile, tert-butylmethylether, acetone and ethanol p. a. were from E. Merck K G (Darmstadt, Germany). Ammonium acetate p.a., butylated hydroxy toluene p.a., tetrahydrofuran p.a., triethylamine p.a., were from Fluka Chemie A G (Buchs, Switzerland). HPLC grade solvents for stock solutions, dilutions, and sample solvent mixtures were additionally purified over basic aluminum oxide grade 1 (Camag, Muttenz, Switzerland). To determine the β-carotene content of cells, the cell layer was washed 5 times with PBS/2% BSA. The cells were detached by trypsin/EDTA 0.05/0.02% and centrifuged at 10,000×g for 1 minute Cell pellets were lysed with acetone containing 0.025% BHT (v/w), vortex-mixed and dried in a speed-vac. The dried residue was extracted with ethanol/tBME/THF, 9:5:1, containing 0.025% of BHT (v/w) by vigorous vortex-mixing for one minute, and centrifugation for 3 minutes at 10,000×g. An aliquot of the clear supernatant was injected into the HPLC system. Cell culture medium was directly extracted with the solvent mixture described above, and the extract was treated as described for the cell pellets.

The HPLC system consisted of a 520 pump, a 565 autosampler cooled at 6° C., a 540+ diode array detector, a SDU 2003 solvent degasser unit and the Chroma 3000 data analysis system from Bio-Tek Instruments (Basel, Switzerland). A Vydac 218TP54 column (250×4.5 mm i.d., 300 angstrom pore wide) from the Separation Group (Hesperia, USA) was used for separation. The mobile phase consisted of acetonitrile/tert.-butylmethylether/aqueous ammonium acetate 80 mM/triethylamine, 73:20:7:0.05, (v/v/v/v) eluted under isocratic condition. The flow rate was adjusted to 1.5 ml/min and the injected sample volume was 25 μl. The effluent was monitored at 325 nm for retinol and retinyl palmitate, 450 nm for β-carotene, and scanned between 190 and 500 nm by the DAD to detect β-carotene-isomers and apocarotenals. Standard solutions from DSM Nutritional Products (Kaiseraugst, Switzerland) in the range of expected sample concentration were used to quantify all-E-β-carotene, (9Z)-β-carotene, (13Z)-β-carotene, 4′-β-apocarotenal, 8′-β-apocarotenal, 12′-3-apocarotenal, all-E-retinol, and retinyl palmitate in cell culture extracts. From the HPLC chromatograms of the standards an average value of the relevant peak areas was divided by the corresponding photometrically: measured concentration in a defined injection volume. This resulted in specific HPLC response factors (RF values) for each compound at defined chromatographic conditions. Limits of β-carotene and apocarotenals quantification (LOQ) were in the range of 0.05-to-0.1 μmol·L⁻¹ for media and 0.6-to-1.0 μmol for 1×10⁶ cells. The limit of detection for retinol and retinyl palmitate was below 0.5 μmol for 1×10⁶ cells.

The identification of the major β-carotene metabolites formed in cells was based on expected elution order as well as on absorption spectra obtained by photodiode array detection. To confirm these results, some cell extracts were analysed by APCI⁺ tandem mass spectrometry. The main metabolites formed were identified as (13Z)-β-carotene (m/z:536), 4′-β-apocarotenal (m/z:482), 8′-β-apocarotenal (m/z:416) and monoepoxy-β-carotene (m/z:620). In addition, a number of minor, yet unresolved, peaks were detected between 360 and 450 nm. Since the expected amount of RA was below the limit of detection, we used an RARE-driven reporter construct to indirectly measure retinoid activity (see below).

### RNA Isolation and Quantitative RT-PCR (QRT-PCR)

Total RNA was isolated by using Trizol™ (Invitrogen, Basel, Switzerland) according to the instructions of the manufacturer. Random-primed cDNA was synthesized using the Superscript pre-amplification system for first strand cDNA synthesis (Invitrogen).

cDNA corresponding to 10 ng total RNA was used as template to quantify the relative RNA expression of the genes of interest by TaqMan® real time PCR. The sequences of the primers and probes are shown in Table 1.

TABLE 1
Primers and probes used for QRT-PCR.

| | | | | Basal |
|---|---|---|---|---|
| Tran- | Forward | Reverse | | Expres- |
| script | Primer | Primer | Probe | sion |
| MMP-1 | AGATGAAA | CCAAGAG | AGAGAGTACAA | 12.67± |
| | GGTGGACC | AATGGCC | CTTACATCGTG | 0.57 |
| | AACAATTT | GAGTTC | TTGCGGCTCA | |
| | (SEQ ID | (SEQ ID | (SEQ ID | |
| | NO: 1) | NO: 2) | NO: 3) | |
| MMP-3 | Hs00233962 m1 Assay-on-Demand | | | 22.80± |
| | (Applied Biosystems) | | | 0.70 |
| MMP-10 | AACAGATTTTG | TTCGCAAGATG | AGGCAGGGGG | 15.35± |
| | TGGGCACCA G | ATGTGAATGG | AGGTCCGTAG | 0.569 |
| | (SEQ ID | (SEQ ID | AGAGACT | |
| | NO: 4) | NO: 5) | SEQ ID | |
| | | | NO: 6) | |
| MMP-2 | CCCTCGCA | CAGATCAGGTG | TGGGACAAGA | 18.64± |
| | AGCCCAA | TGTAGCCAATG | ACCAGATCAC | 1.35 |
| | (SEQ ID | (SEQ ID | ATACAGGA | |
| | NO: 7) | NO: 8) | (SEQ ID | |
| | | | NO: 9) | |
| MMP-9 | CCTGAGAACCA | GCCACCCGAGT | AGGCAGCTGG | 21.08± |
| | ATCTCACCG A | GTAACCATAG | CAGAGGAATA | 0.89 |
| | (SEQ ID | (SEQ ID | CCTGTACC | |
| | NO: 10) | NO: 11) | (SEQ ID | |
| | | | NO: 12) | |
| TIMP-1 | CACCCACAG | CTGGTGTCCC | CCCTGATGAC | 5.90± |
| | ACGGCCTTC | CACGAACTTG | GAGGTCGGAA | 2.40 |
| | (SEQ ID | SEQ ID | TTGC | |
| | NO: 13) | NO: 14) | (SEQ ID | |
| | | | NO: 15) | |
| P- | AGGAAAGAACA | GTTCCCTGC | TGAGGGCCA | 23.76± |
| carotene | GCTGGAGC CT | AGCCATGCT | AAGTGACAG | 1.27 |
| 15,15′- | (SEQ ID | (SEQ ID | GCAAGATT | |
| | NO: 16) | NO: 17) | (SEQ ID | |
| | | | NO: 18) | |
| p-carotene | GCTCAATGGCT | CAGCGCCAT | CGAGTTTG | 19.28± |
| 9′,10′- | CTCTACTTCGM | CCCATCAA | GGAAGGAT | 0.469 |
| oxygenase | (SEQ ID | (SEQ ID | AAGTACAA | |
| | NO: 19) | NO: 20) | TCATTGG | |
| | | | (SEQ ID | |
| | | | NO: 21) | |
| RARa | GTCCTCAG | TGTACACCA | CTGCAAGG | 9.81± |
| | GCTACCAC | TGTTCTTCT | GCTTCTTC | 1.29 |
| | TATGGG | GGAT GC | CGCCGCA | |
| | (SEQ ID | (SEQ ID | (SEQ ID | |
| | NO: 22) | NO: 23) | NO: 24) | |
| RARP | AAATCAT | CGGTGACA | CTGTGAGG | 14.49± |
| | CAGGGTA | AGTGTAAA | GATGTAAG | 0.44 |
| | CCACTAT | TCATATTC | GGCTTTTT | |
| | GGG | TTC | CCGC | |
| | (SEQ ID | (SEQ ID | (SEQ ID | |
| | NO: 25) | NO: 26)_ | NO: 27) | |
| RARy | GTTCTTC | GTCTACAA | AAGAAGCC | 13.20± |
| | TGGATGC | GCCATGC | CTTGCAGC | 0.46 |
| | TTCGGC | TTCGTGT | CTTC ACA | |
| | (SEQ ID | (SEQ ID | (SEQ ID | |
| | NO: 28) | NO: 29) | NO: 30) | |
| RXRa | AAGCACA | TGCACCCC | ACCGCTCCT | 9.73± |
| | TCTGCGC | TCGCAGCT | CAGGCAAG | 0.71 |
| | CATCT | (SEQID | CACTATGG | |
| | (SEQ ID | NO: 32) | (SEQ ID | |
| | NO: 31) | | NO: 33) | |
| RXRP | TCTGGATG | TCGGTGT | CGGGCAGGC | 12.97± |
| | ATCAGGTC | GAAAAGG | TGGAATGA | 0.486 |
| | ATATTGCT | AGGCA | ACT CCTC | |
| | (SEQ ID | (SEQ ID | (SEQ ID | |
| | NO: 34) | NO: 35) | NO: 36) | |
| RXRy | GCCTCCA | TTGATGTC | CCACCCAGCAT | 17.90± |
| | GGAATCA | CTCTGAAC | CTCAGCTAAA | 1.53 |
| | ACTTGG | TGCTGA C | TGTGGTCA_ | |
| | (SEQ ID | (SEQ ID | (SEQ ID | |
| | NO: 37) | NO: 38) | NO: 39) | |
| 185 | CGGCTAC | GCTGGAA | TGCTGGCAC | 0 |
| rRNA | CACATCC | TTACCGC | CAGACTTGC | (base- |
| | AAGGAA | GGCT | CC TC | line) |
| | (SEQ ID | (SEQ ID | (SEQ ID | |
| | NO: 40) | NO: 41) | NO: 42) | |

The PCR analyses were carried out in triplicate and in a multiplex setup, using 18S rRNA as a calibrator gene. The rRNA primers were used at a final concentration of 50 nM, the probe at 100 nM. For quantification of the genes of interest, the primer concentrations were optimized for sensitivity of template detection. Moreover, it was verified that the amplification of the calibrator gene did not interfere with the detection of the gene of interest. PCR reactions were carried out for 40 cycles of 95° C. for 15 seconds and 60° C. for 1 minute in an ABI7700 (Applied Biosystems, Rotkreuz, Switzerland). Regulation of gene expression was calculated as described in user bulletin #2 provided by the manufacturer. A threshold cycle (“CT”) is the first PCR cycle in which an amplification signal is detected. Expression levels are given as δCT values. The δCT value describes the level of gene expression as the average PCR cycle, in which the gene of interest was detected first (CT value), subtracted by the CT value of the calibrator gene 18S rRNA (CT_{gene} of interest−CT_{calibrating gene}), 18S rRNA served as a measure for the amount of template in the reaction. Routinely, 18S rRNA was detected between PCR cycle 12 and 13 (SE=standard error) A low δCT corresponds to a high mRNA level.

Treatment-induced gene regulations are given as fold change relative to the placebo-treated controls. Transcripts were classified as low abundant, if the δCT value was below 23. This expression level is the approximate limit of quantification of the method. Transcripts were called ‘medium abundant’, if their δCT was between 23 and 13. Transcripts detected earlier than at a δCT of 13 were categorized as high abundance transcripts.

### ELISA

Release of MMP-1, and TIMP-1 into cell culture supernatant was determined by ELISA at 24 hours after irradiation. MMP-1 and TIMP-1 release was measured using MMP-1 and TIMP ELISA from CALBIOCHEM (San Diego, Calif., USA). The ELISAs were performed according to the manufacturer's instructions.

### Reporter Gene Assay

HaCaT cells were seeded at a density of 3×10⁶ cells/well in 6 well plates (BD Biosciences, Basel, Switzerland) in FAD medium containing 2% NuSerum. The cells were transfected the next day using 1 μg reporter plasmid (pGL3 (RARE)5 tk luc) and 5 μl Lipofectin (1 μg/μL; Invitrogen, Basel, Switzerland) per well. The RARE is a DR5 element identical to the wild type element of the RARβ2 promoter. The spacing between the DR5 sites is 25 nucleotides. The transfections were performed for 7.5 hours in serum-free FAD medium according to the manufacturer's protocol. The transfections were stopped by replacing the media with FAD/2% NuSerum, or FAD/2% NU serum containing 1 or 3 μM β-carotene, respectively. The solvent concentration was 0.5% THF/ethanol (1:1) for all media. The cells received fresh media the next day. Transactivation of the reporter gene was determined after 40 hours of β-carotene treatment.

To generate the RA standard curve, 9-cis RA and all-trans RA were used together at concentrations ranging from 10⁻¹⁰ to 10⁻⁸ M each. Forty hours later, the cells were washed 6 times with PBS/2% BSA. Cells were irradiated as described above. To prepare cell extracts, the cells were detached from the culture dishes by trypsinisation, and washed in PBS. The cell pellets were dissolved in 500 μl of 0.1 M KHPO₄, and the cells were disrupted by three freeze/thaw cycles. Relative luciferase units (RLU) were quantified in a luminoskan reader (Thermo Labsystems, Vantaa, Finland), and corrected by protein concentrations as determined with the BCA assay (Pierce, Rockford, USA).

### Statistical Analysis

The results were analyzed for significant treatment effects by ANOVA. If the ANOVA returned a P value below 0.05, the treatment effect was considered significant. Only significant effects were further analyzed by the post-hoc test Fisher's PLSD test, to allow for multiple pair-wise comparisons of treatment conditions, and to detect dose-dependent effects. Again, effects with a P value below 0.05 were regarded as significant. The statistical analysis was done using the software package Statview (SAS Institute Inc., Cary, USA).

### Results

### Time- and Dose-Dependent Accumulation of β-Carotene in HaCaT Cells

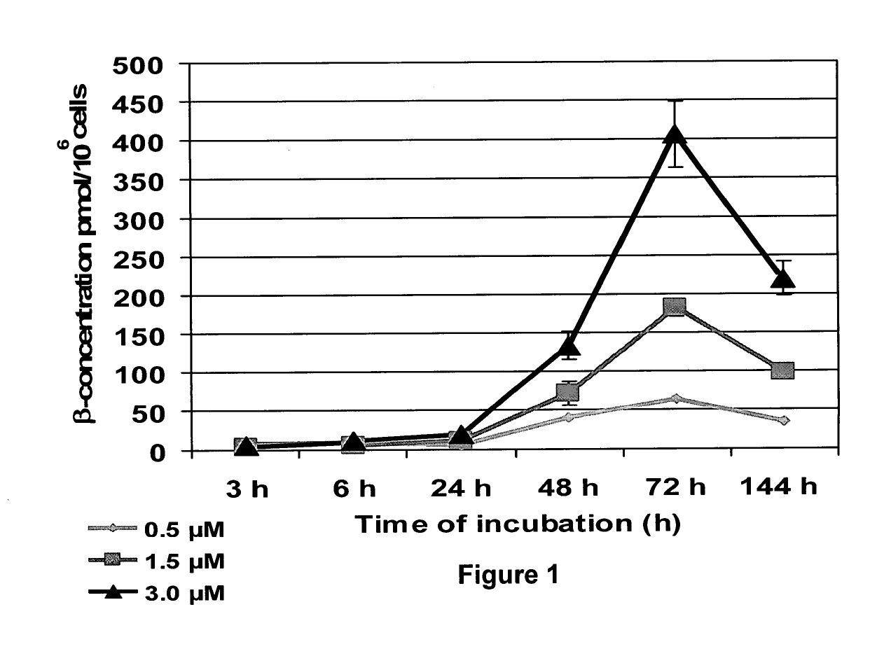

HaCaT cells were supplemented with β-carotene-containing medium for 3, 6, 24, 48, 72, or 144 hours and subsequently analyzed for their β-carotene content by HPLC analysis. β-carotene was time-dependently accumulated in HaCaT cells, with the peak β-carotene concentration being achieved after 72 hours of supplementation (FIG. 1). After this time point, when the cells were kept for an additional 3 days without adding fresh β-carotene-containing media, the β-carotene contents dropped to about half the concentration observed at 72 hours. The β-carotene concentration in cells was dose-dependent, and increased from 63 μmol/million cells at 0.5 μM to 406 μmol/million cells at 3.0 μM within a culture period of 72 hours.

### UVA Irradiation Lead to Depletion of the Cellular β-Carotene Content

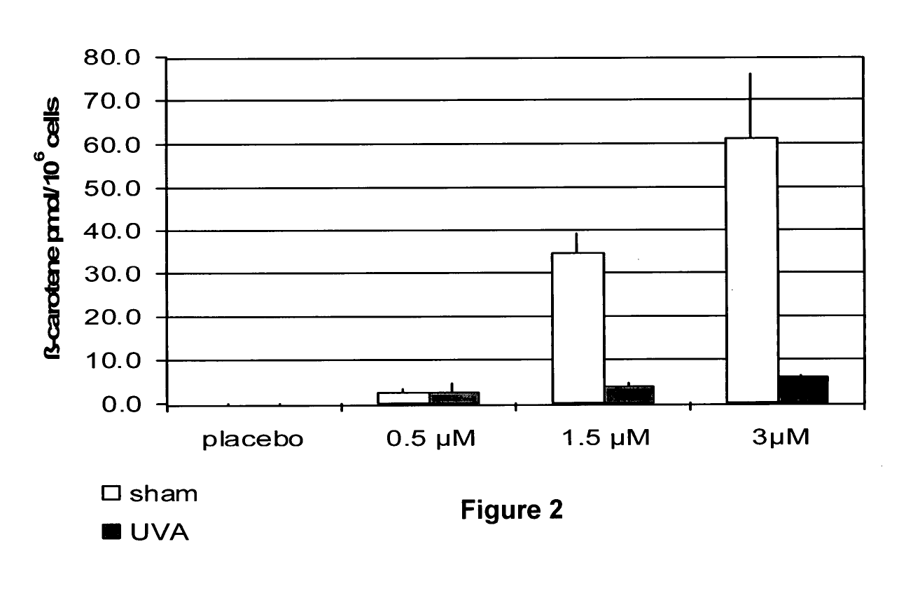

Cells supplemented with 0.5, 1.5, or 3 μM β-carotene for 2 days, were irradiated with 270 kJ/m² UVA, to determine the effect of irradiation on the β-carotene content of the cells. UVA irradiation diminished the β-carotene stores to about 13% in cells incubated in 1.5 or 3 μM β-carotene (FIG. 2). UVA did not reduce the cellular β-carotene content after incubation with 0.5 μM β-carotene.

### β-Carotene Reduced UVA-Induced MMP-1, MMP-3, and MMP-10 Induction

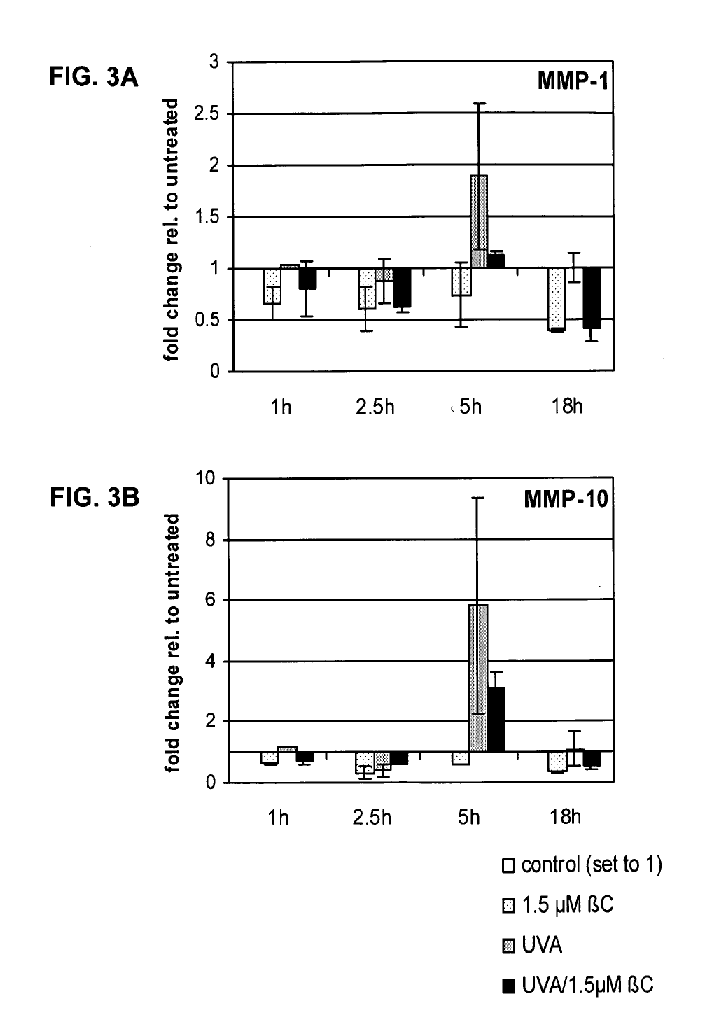

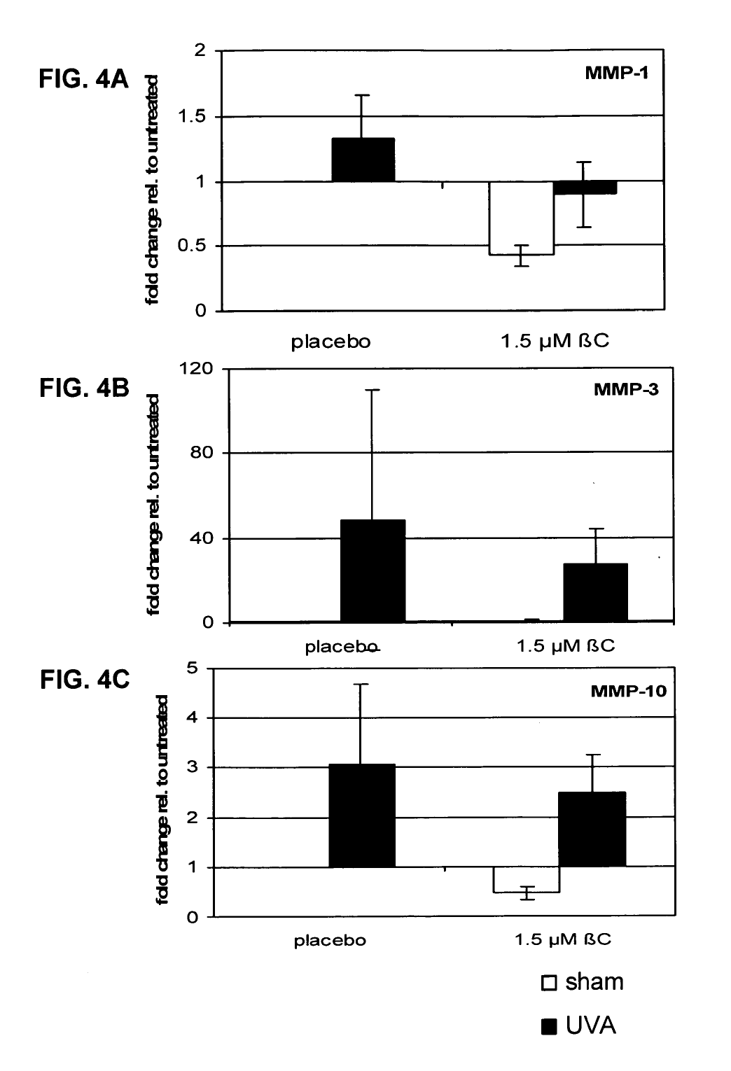

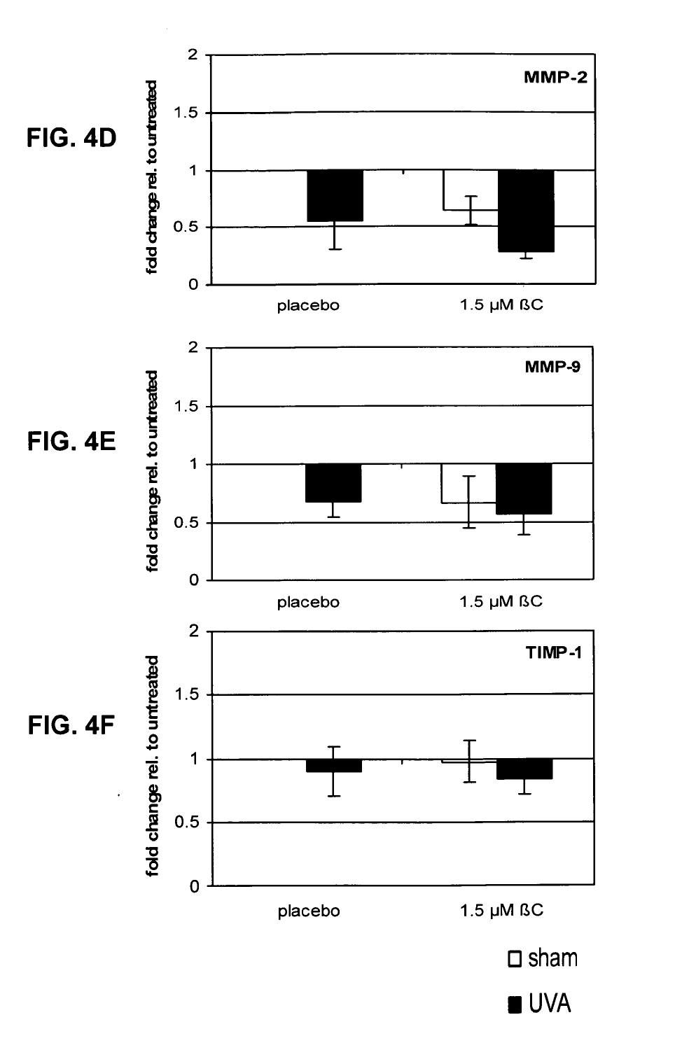

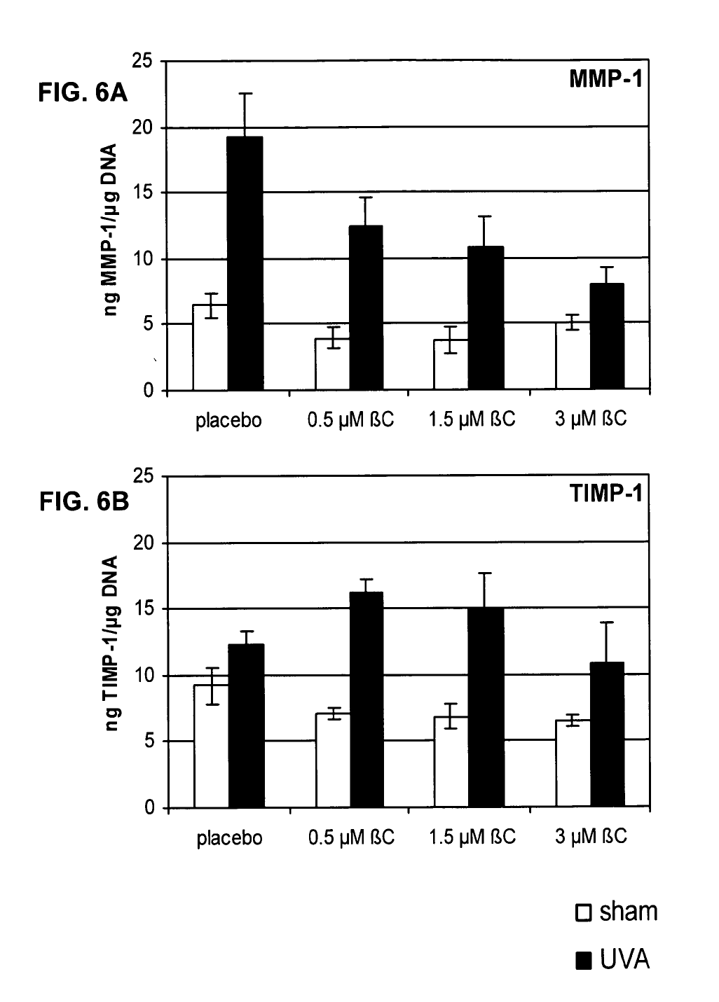

β-Carotene, like other carotenoids, is an excellent ¹O₂ quencher [66, 67]. Since ¹O₂-dependent induction of MMPs upon UVA exposure is thought to be a major mechanism of photoaging, β-carotene inhibition of MMP induction upon UVA exposure was measured. Among MMPs, MMP-1 is best characterized in terms of induction by UV light, and is the most accepted marker for photoaging. MMP-1 transcripts were present at medium to high levels in HaCaT cells, and were detected at a δCT of 12.67 in controls. We found a 2.4 fold (SE±0.7) induction of MMP-1 by UVA at 5 hours after irradiation, but only little inducibility at the other time points analyzed (FIG. 3 a). Therefore, the 5-hour time point was chosen to analyze the effect of treatments on gene expression in all further experiments. The degree of UVA inducibility of MMP-1 expression varied between experiments. In any case, β-carotene at a concentration of 1.5 μM significantly reduced UVA-induced MMP-1 induction from 1.3 fold to 0.9 fold on average (FIG. 4 a; P=0.047). Downregulation of UVA-induced MMP-1 production by β-carotene was also confirmed on the protein level (FIG. 6 a; ANOVA P=0.005).

Microarray analysis [53] showed that among the MMP genes detected by the array MMP-10 (stromelysin-2) was the most strongly induced by UVA in HaCaT cells at 5 hours after irradiation. Pretreatment with 1.5 μM β-carotene moderately reduced UVA induction of MMP-10 by 30% from 4.6 fold to 3.2 fold. This result was confirmed by QRT-PCR. MMP-10 was a medium abundant transcript in untreated HaCaT cells (δCT 15.35). UVA induced MMP-10 expression to about 3 fold relative to the expression in unirradiated cells (FIG. 4 c). 1.5 μM β-carotene reduced UVA induction of MMP-10 to approx. 2.5 fold, an effect that reached marginal significance (P=0.088). As with MMP-1, MMP-10 was maximally induced 5 hours after UVA irradiation (FIG. 3 b; UVA effect at 5 hours P<0.0001). UVA exposure increased MMP-10 expression 5.8 fold (SE±3.56) at this time point.

MMP-3 (stromelysin-1) was analyzed as a close relative to MMP-10. MMP-3 was present at medium abundance in HaCaT cells (δCT 22.8). MMP-3 is also known to be induced by UVA1 light (340-450 nm) [68, 69]. Accordingly, MMP-3 was induced approx. 49 fold by UVA exposure, and 1.5 μM β-carotene non-significantly reduced UVA-induction of MMP-3 to 27 fold relative to unirradiated controls (FIG. 4 b).

The expression profiles of the two gelatinases, MMP-2 and MMP-9, were analyzed. MMP-9 is induced by UV irradiation in skin [70]. Both MMP-2 and MMP-9 were expressed at medium abundance with δCTs of 18.64 and 21.08, respectively, in controls. Unexpectedly, neither of the gelatinases was induced by this irradiation regimen, and β-carotene did not influence their expression significantly (FIGS. 4 d and 4 e).

TIMP-1, an endogenous MMP inhibitor, was strongly expressed (δCT 5.9), but not significantly influenced by the treatments on the RNA (FIG. 4 f) or protein level (FIG. 6 b).

β-Carotene Acted as a ¹O₂-Quencher in Living Cells

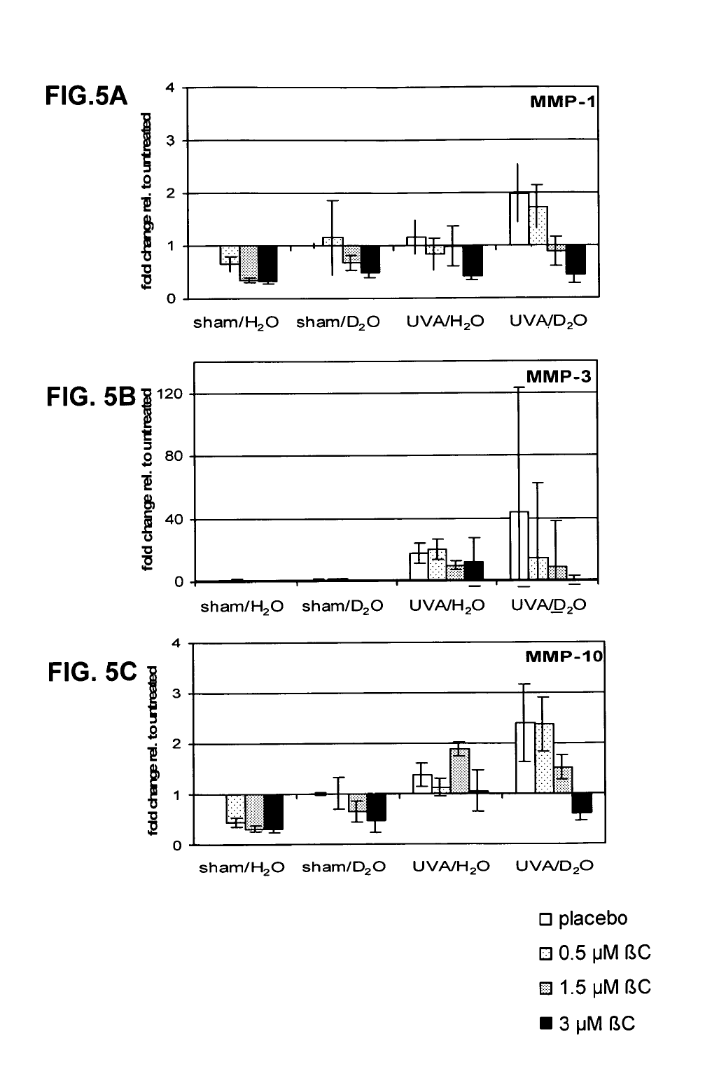

To test whether the mechanism by which β-carotene interferes with UVA induction of MMPs involves ¹O₂ quenching, cells were irradiated either in D₂O-containing buffer or in H₂O-containing buffer. D₂O is able to prolong the lifetime of ¹O₂ [18]. Thus, the probability of ¹O₂ reacting with a relevant target is increased. Accordingly, ¹O₂-dependent MMP induction upon UVA exposure should be more pronounced after irradiation in the presence of D₂O. β-carotene should then be able to reduce MMP induction by UVA/D₂O treatment. Wlaschek et al. [13, 15, 71] have described that MMP-1 induction by UVA involves ¹O₂-dependent mechanisms. In line with this, QRT-PCR analysis indeed revealed greater induction of MMP-1, when the cells were irradiated in the presence of D₂O (FIG. 5 a; 1.9 fold vs. 1.2 fold; ANOVA P for D₂O effect=0.0505; Fisher's PLSD test D₂O vs. H₂O P=0.0011). β-carotene significantly and dose-dependently reduced UVA/D₂O-induced MMP-1 induction (ANOVA P for β-carotene effect=0.0563; Fisher's PLSD: β-carotene at 1.5 μM P=0.0405; β-carotene at 3 μM P<0.0001). Moreover, β-carotene treatment also tended to reduce basal MMP-1 RNA and protein expression in unirradiated cells (Protein: FIG. 6 a; P=0.0537).

MMP-10 is known to be induced by UV light [72]. So far, it has not been demonstrated whether MMP-10 regulation also involves ¹O₂-dependent pathways. Provided below is evidence that MMP-10 is also a ¹O₂-regulated gene. D₂O significantly enhanced UVA induction of MMP-10 from 1.4 fold to 2.4 fold relative to unirradiated controls (FIG. 5 c; ANOVA P for D₂O effect=0.0017; Fisher's PLSD H₂O vs. D₂O P=0.0004). This shows that MMP-10 induction by UVA irradiation involves ¹O₂-dependent mechanisms.

Pretreatment of cells with different doses of β-carotene opposed MMP-10 induction by UVA and D₂O in a dose-dependent fashion (ANOVA P for β-carotene effect=0.0368; Fisher's PLSD β-carotene at 3 μM P=0.0021). Like for MMP-1, β-carotene also tended to reduce the basal MMP-10 expression in unirradiated cells.

Both the expression profiles of MMP-1 and MMP-10 prove that β-carotene can act as a ¹O₂ quencher in living cells.

MMP-3 induction by UVA was enhanced by irradiation in D₂O-containing buffer from 18 fold to 43 fold (FIG. 5 b). Since the degree of MMP-3 inducibility by UVA/D₂O varied between experiments, the effect of D₂O did not reach significance (ANOVA P=0.24). Although it remains unclear, whether MMP-3 regulation includes ¹O₂-dependent mechanisms, β-carotene prevented MMP-3 induction by UVA irradiation in the presence or absence of D₂O (ANOVA P=0.04; Fisher's PLSD P for β-carotene 3 μM=0.007).

MMP-2 and MMP-9 were not induced by UVA/D₂O treatment, and β-carotene had no significant effect on their expression (data not shown). For MMP-9, β-carotene tended to lower expression in irradiated and unirradiated cells (ANOVA P for β-carotene effect=0.08; Fisher's PLSD P for β-carotene 1.5 μM=0.028; P for β-carotene 3 μM=0.012).

TIMP-1 was again not significantly influenced by the treatments (data not shown).

Vitamin E Did not Synergize with β-Carotene to Further Reduce MMP-1, MMP-3, or MMP-10 Expression

The chain-breaking antioxidant Vitamin E is thought to protect the ¹O₂ quencher β-carotene from destruction by other reactive oxygen species, and is therefore expected to potentiate the effect of β-carotene [73]. Thus, whether vitamin E at the physiologically: relevant concentration of 50 μM synergizes with β-carotene at 1.5 μM to reduce MMP-1, MMP-3, and MMP-10 expression was tested. In at least four independent experiments, vitamin E did not cooperate with β-carotene in reducing UVA-induction of MMP-1, MMP-3, or MMP-10. In fact, vitamin E alone showed no effect on MMP-1, MMP-3, or MMP-10 expression (data not shown). TIMP-1 expression was reduced by UVA in this set of experiments (P=0.0008). UVA-suppressed TIMP-1 expression was restored by vitamin E (P=0.016; data not shown).

Weak Retinoid Activity is Generated from β-Carotene in HaCaT Cells and Reduced by UVA

By HPLC analysis, we found that HaCaT keratinocytes do not produce detectable amounts of retinol or retinyl esters from β-carotene. In contrast, apocarotenals were detected. HaCaT cells were treated with 0.5, 1.5, or 3 μM β-carotene for 2 days. Cellular contents of β-carotene and β-carotene metabolites were quantified by HPLC. The results are reported in Table 2. The cellular apocarotenal contents increased dose dependently, and amounted to maximum 5 μmol/million cells treated with 3 μM β-carotene. Moreover, a fraction of the supplemented all-E β-carotene was isomerized to (Z) isomers. The amount of (Z) isomers also increased dose-dependently, and was maximum 0.8 pmol/million cells after supplementation with 3 μM β-carotene.

TABLE 2
β-Carotene uptake and metabolism m HaCaT cells.

| P-Carotene | aii-E-P- | (Z)-13- | | | Retinyl |
|---|---|---|---|---|---|
| Supplementation (IJM) | Carotene | Carotene | Apocarotenals | Retinol | Palmitate |
| placebo | <LOD | <LOD | <LOD | <LOD | <LOD |
| 0.5 | 9.7 ± 0.09 | 0.2 ± 0.07 | 1.18 ± 0.04 | <LOD | <LOD |
| 1.5 | 34.3 ± 0.05 | 0.41 ± 0.02 | 3.21 ± 0.19 | <LOD | <LOD |
| 3.0 | 63.90 ± 0.22 | 0.82 ± 0.16 | 5.04 ± 0.11 | <LOD | <LOD |

(pmol/10⁶ cells) (LOD—below limit of detection.)

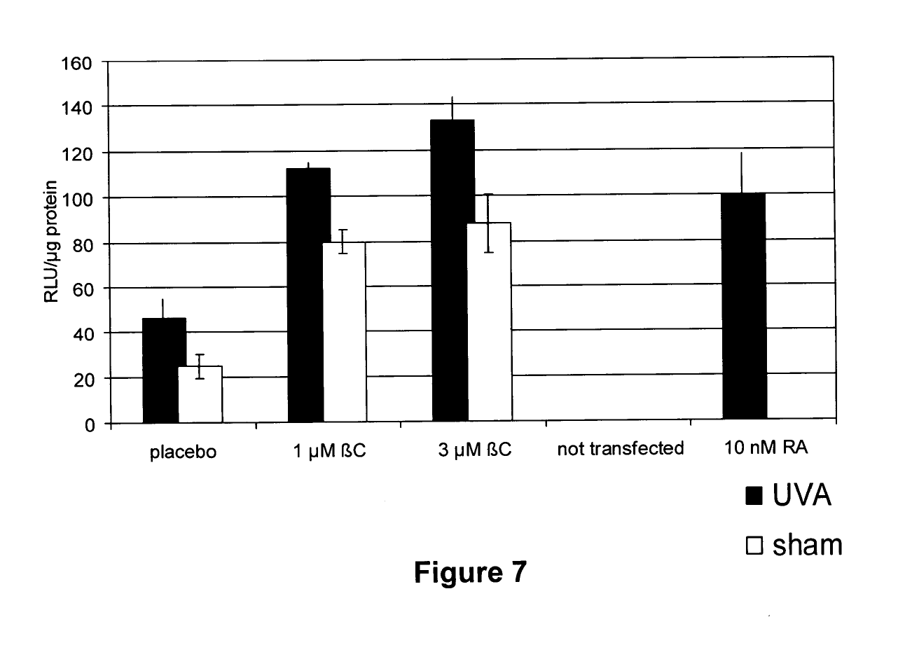

Despite undetectable retinol formation from β-carotene, RA was formed after β-carotene treatment, as shown by transactivation of an RA-dependent reporter gene (FIG. 7). Treatment of HaCaT cells with 1 or 3 μM β-carotene caused activation of the luciferase reporter to a degree comparable to what was achieved after treating the cells with a combination of all-trans RA and 9-cis RA at 10 nM each. RARE-dependent gene activation by β-carotene was reduced to about 70%, if the cells were irradiated with UVA prior to the activation measurement.

Next, results were correlated on β-carotene metabolism and RA-dependent gene activation with the expression profiles of the two β-carotene cleavage enzymes and the nuclear receptors responsible for transducing the RA effect on gene expression.

β-Carotene-15,15′-oxygenase[74-77] cleaves β-carotene centrally to yield retinal. β-Carotene-15,15′-oxygenase was expressed at a relatively low level with a δCT of 23.8 in controls. Transcripts for β-carotene-9′,10′-oxygenase[78], which produces 10′-apocarotenal and β-ionone from β-carotene[78], were present at about 23 fold higher abundance δCT 19.3). The RNA levels of both enzymes were not significantly influenced by the treatments.

HaCaT cells were pretreated for 2 days with 0.5, 1.5 or 3 μM β-carotene. The cells were irradiated with UVA (270 kJ/m²) either in D₂O-containing PBS or in H₂O-containing PBS, to analyze ¹O₂ inducibility of genes. Gene expression 5 hours after UVA irradiation was analyzed by QRT-PCR. The results are reported in Table 3. Values are geometric means±standard error from three independent experiments. Upregulations greater than 1.5-fold are labelled in bold black, downregulations below 0.66-fold are bold grey.

TABLE 3
Fold induction effect of β-carotene on expression of retinoid
receptors after UVA or D₂O-enhanced UVA irradiation.

| | H₂O | | | | | | | |
|---|---|---|---|---|---|---|---|---|
| Retinoid | | | UVA/βC | UVA/βC | UVA/βC | βC | βC | βC |
| Receptor | Control | UVA | 0.5 μM | 1.5 μM | 3 μM | 0.5 μM | 1.5 μM | 3 μM |
| RARα | 1.00 | 0.97 | 1.45 | 0.47 | 0.71 | 0.56 | 0.67 | 0.55 |
| RARβ | 1.00 | 0.38 | 0.44 | 0.90 | 0.86 | 0.81 | 1.20 | 1.84 |
| RARγ | 1.00 | 0.51 | 0.85 | 0.15 | 0.54 | 0.74 | 0.86 | 0.90 |
| RXRα | 1.00 | 0.57 | 0.96 | 0.27 | 0.58 | 0.89 | 0.98 | 1.13 |
| RXRβ | 1.00 | 0.63 | 0.93 | 0.71 | 1.04 | 0.67 | 0.99 | 0.99 |
| RXRγ | 1.00 | 0.45 | 0.61 | 0.08 | 0.67 | 0.39 | 0.77 | 0.33 |
| βC-15,15′- | 1.00 | 0.61 | 0.60 | 0.84 | 0.91 | 1.09 | 1.19 | 1.08 |
| oxygenase | | | | | | | | |
| βC-9′,10′- | 1.00 | 0.56 | 0.75 | 0.70 | 0.99 | 0.90 | 0.85 | 0.76 |
| oxygenase | | | | | | | | |
| | D₂O | | | | | | | |
| Retinoid | | | UVA/βC | UVA/βC | UVA/βC | βC | βC | βC |
| Receptor | Control | UVA | 0.5 μM | 1.5 μM | 3 μM | 0.5 μM | 1.5 μM | 3 μM |
| RARα | 1.00 | 1.58 | 1.06 | 1.21 | 0.52 | 1.01 | 1.02 | 1.18 |
| RARβ | 1.00 | 1.15 | 2.49 | 1.69 | 2.35 | 1.25 | 2.46 | 2.79 |
| RARγ | 1.00 | 1.39 | 0.63 | 0.67 | 0.76 | 0.94 | 1.11 | 1.12 |
| RXRα | 1.00 | 0.79 | 0.68 | 0.31 | 0.38 | 1.25 | 0.82 | 1.06 |
| RXRβ | 1.00 | 1.32 | 1.23 | 1.09 | 0.76 | 1.32 | 1.17 | 1.61 |
| RXRγ | 1.00 | 3.17 | 1.72 | 2.70 | 1.57 | 1.47 | 2.78 | 2.57 |
| βC-15,15′- | 1.00 | 0.58 | 1.08 | 0.90 | 0.91 | 1.19 | 1.03 | 0.43 |
| oxygenase | | | | | | | | |
| βC-9′,10′- | 1.00 | 1.45 | 1.43 | 2.30 | 2.19 | 0.93 | 1.19 | 1.11 |
| oxygenase | | | | | | | | |

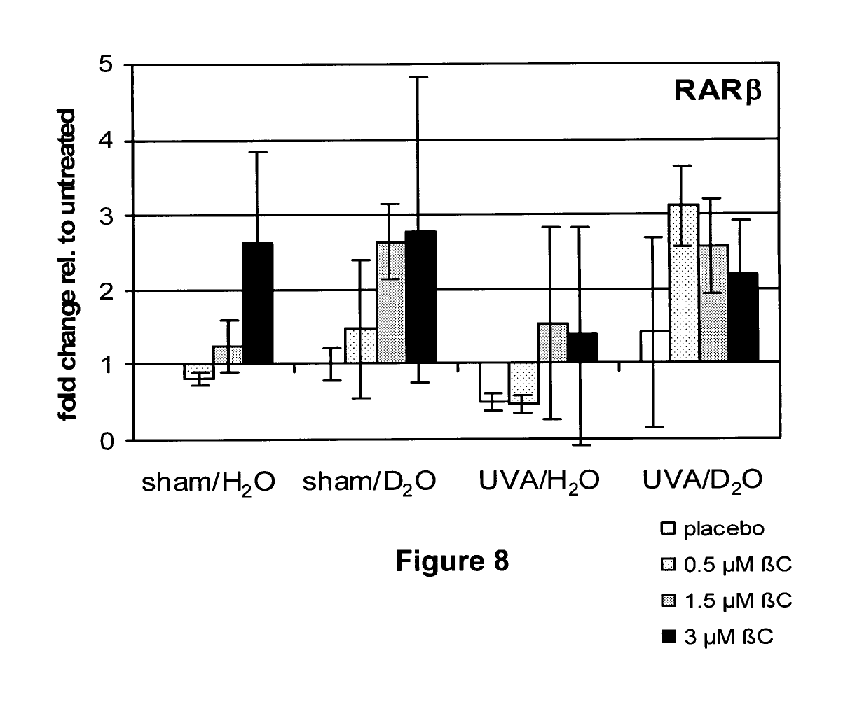

Expression of all six retinoid receptor genes (RARα, RARβ, and RARγ and RXRα, RXRβ, and RXRγ) was detected in HaCaT cells. RXRα was expressed the strongest among retinoid receptors with a δCT of 9.7 in controls, followed by RARα(9.8), RXRβ(13.0), RARγ(13.2), RARβ(14.5), and RXRγ(17.9). UVA downregulated all retinoid receptors approximately 2-fold, except for RARα, which was not influenced by UVA. UVA downregulation of RARs and RXRs reached significance only for RXRα. Apparently, regulation of RARα and γ expression, as well as regulation of RXRα and γ has a ¹O₂-dependent component, as D₂O treatment had a significant effect on these transcripts. β-Carotene had no significant effect on the basal or UVA-regulated expression levels of RARs and RXRs. Of note, β-carotene non-significantly induced RARβ in a dose-dependent manner, an effect observed predominantly in unirradiated cells (FIG. 8).

### Conclusion

The ¹O₂ quencher β-carotene alleviates UVA induction of MMP-1, MMP-3, and MMP-10, three major metalloproteases involved in premature skin aging. Moreover, the β-carotene effects were exerted mainly via RA-independent pathways. HaCaT cells produce low amounts of RA from β-carotene, as shown by monitoring RA-dependent gene activation. Thus, HaCaT cells are an excellent model to analyze the provitamin A-independent effects of β-carotene.

### Time- and Dose-Dependent Accumulation of 11-Carotene in HaCaT Cells

HaCaT keratinocytes took up β-carotene in a time: and dose-dependent manner (FIG. 1). HaCaT cells had to be supplemented at least for two days to achieve meaningful β-carotene accumulation. The cells continued to take up β-carotene thereafter, such that maximum β-carotene levels were found after three days of supplementation. After that, daily supplementation was ceased, to monitor the cellular β-carotene content over time, if no fresh β-carotene was added. As a result, β-carotene decreased, demonstrating that a daily supply of fresh β-carotene is critical to maintain cellular β-carotene content.

### UVA Irradiation Depleted Cellular β-Carotene Content

The UVA dose applied destroyed all β-carotene but about 13% of the content before irradiation, which confirms similar reports from β-carotene supplemented fibroblasts after UVA exposure [79] (FIG. 2). Consistent with this finding, RARE-dependent gene activation by β-carotene was reduced, if the cells were irradiated with UVA (FIG. 7). These results are in line with in vivo observations that UVA exposure depletes epidermal vitamin A stores [80]. Moreover, UVA irradiation reduces carotenoid concentrations in skin [24] and even in plasma [81]. In view of the role of vitamin A in maintaining skin integrity, depletion of vitamin A and provitamin A stores by UV light calls for increased vitamin A uptake in situations with extensive sun exposure.

β-Carotene Reduced Basal and ¹O₂-Induced MMP-1 and MMP-10 Induction

According to the current model of photoaging [45], UV irradiation activates growth factor and cytokine receptors, which via PKC, MAP kinases, and the NFκB pathway activate genes involved in photoaging, such as MMPs [82]. UVA1, on the other hand, is thought to induce genes associated with photoaging by ¹O₂-mediated pathways that target the transcription factor AP-2 [16]. Of the genes involved in photoaging, MMP-1 [12, 71], IL-6 [71], and ICAM-1 [16] have been shown to be induced in a ¹O₂-dependent fashion upon UVA exposure. Other reports suggest that the cellular reaction to UVA1, like UVB/UVA2, also includes activation of the stress-activated protein kinases [β-85]. Therefore, the response to UVA1 vs. UVB/UVA2 exposure, and the pathways involved, overlap. The extent to which the MMPs mainly responsible for extracellular matrix degradation are transcriptionally regulated by ¹O₂ exposure (i.e. UVA/D₂O treatment), and how the ¹O₂ quencher β-carotene would interfere with this regulation, were investigated.

That UVA induction of MMP-1 involves a ¹O₂-dependent mechanism in keratinocytes was confirmed. β-Carotene inhibited UVA/D₂O-induced MMP-1 expression in a dose-dependent manner to below control levels, demonstrating that under appropriately controlled conditions, β-carotene acts as a ¹O₂ quencher also in living cells (FIG. 5). Our results are in contrast to those reported by Obermuller-Jevic et al. [86, 87], and Offord et al. [88]. Both groups have addressed the effect of β-carotene on MMP-1 or HO-1 induction by UVA in fibroblasts. In these studies, no photoprotective effect of β-carotene was found. Rather, β-carotene enhanced UVA-induced MMP-1 and HO-1 induction. On the other hand, Trekli et al. [79] found a photoprotective effect of β-carotene against UVA irradiation in fibroblasts, as determined by HO-1 expression. These contradicting results exclude a fibroblast-specific effect, and point to experimental differences, most likely the mode of β-carotene application. In the studies, where a prooxidative effect of β-carotene was described, β-carotene was delivered to the cells either in methyl-β-cyclodextrin [86, 87], or as a nanoparticle formulation containing vitamin E [88]. Both studies, where β-carotene was photoprotective, THF containing 0.025% BHT was used as a vehicle for β-carotene[79]. A likely explanation for the different experimental outcomes is that BHT protected β-carotene better than the much lower concentration of vitamin E in the nanoparticle formulation. In the studies by Obermüller et al., β-carotene was added to the cells without antioxidant protection. In addition, the vehicle methyl-β-cyclodextrin used by Obermuller et al. is known to remove cholesterol from the cell membranes [89, 90], with drastic consequences for cell signaling events. Although it appears that the major difference is the use of BHT-containing solvent for β-carotene, the presence of the photoprotective effect of β-carotene in the present studies was not due to the protection of β-carotene by BHT. But rather that replacement with fresh β-carotene-containing medium each day and after irradiation was crucial to remove β-carotene degradation products.

Further support for a photoprotective effect of β-carotene comes from the finding that β-carotene protects against mitochondrial common deletions, a mitochondrial DNA mutation, which is induced by repeated UVA irradiation and is associated with photoaging [91]. Protection of fibroblasts against UVB irradiation by β-carotene was demonstrated by Eichler et al. [92].

MMP-10 is a ¹O₂-induced gene (FIG. 5 c). As with MMP-1, β-carotene dose-dependently inhibited UVA/D₂O-induced MMP-10 induction. For both MMP-1 and MMP-10, β-carotene also tended to reduce expression in unirradiated cells, pointing towards a preventive role of β-carotene against intrinsic skin aging. MMP-10 was more strongly induced by UVA than was MMP-1. However, the overall expression profiles of MMP-1 and MMP-10 were remarkably similar, indicating co-regulation. This is in contrast to the RNA expression profiles of the two gelatinases MMP-2 and MMP-9, which were not induced by the irradiation regimen, and which were also not regulated by β-carotene.

The stromelysin MMP-3, which is highly related to MMP-10, was strongly induced by UVA and UVA/D₂O. β-Carotene had a significant reducing effect on MMP-3 expression, although the D₂O effect on MMP-3 induction did not reach significance. The expression profile indicates that MMP-3 regulation may involve ¹O₂-dependent pathways, and Herrmann et al. also suggested that MMP-3 is ¹O₂-inducible [69]. Other mechanisms appear to dominate, however. It is unclear, whether β-carotene reduction of UVA and UVA/D₂O-induced MMP-3 expression was due to its ¹O₂ quenching ability or whether other mechanisms were involved.

Of the three MMPs regulated by UVA and β-carotene, MMP-1 was by far the strongest expressed. MMP-1 mRNA levels were approximately 6 fold higher than those of MMP-10, and 1000 fold higher than those of MMP-3. MMP-1 has a dominant role in UVA-induced degradation of fibrillar collagen, especially of collagen types III and I [93, 94]. Brennan et al. [95] found that blocking antibodies to MMP-1 removed 95% of the collagenolytic activity in the organ culture fluid from UV-treated skin. MMP3 and MMP-10 have broader substrate specificity than MMP-1, and cleave collagen IV, fibronectin, aggrecan and nidogen. Most importantly, both MMP-3 and MMP-10 have an additional role in activating other MMPs, including MMP-1 [93]. Thus, despite their lower expression level in comparison with MMP-1, they have a major impact on ECM degradation. The combined reduction by β-carotene of UVA-induced expression of MMP1, 3, and 10 indicates that β-carotene has a physiologically relevant photoprotective effect.

Vitamin E Did not Synergize with β-Carotene to Further Reduce MMP-1, MMP-3, or MMP-10 Expression

The absence of a synergistic effect of vitamin E and β-carotene may be explained by sufficient amounts of intact β-carotene being present for protection against ¹O₂-mediated MMP induction under our culture conditions, even if some β-carotene was destroyed by oxidative breakdown. The finding that vitamin E alone did not reduce UVA/D₂O-induced expression of any of the MMPs tested is less easily explained, since it has been shown that vitamin E also inhibited some UVA (¹O₂)-induced mechanisms, such as common mitochondrial deletions [96]. Although we did not measure the cellular vitamin E content, it has been shown that HaCaT cells are able to accumulate vitamin E [97], arguing that our findings are not due to a lack of vitamin E uptake. Like β-carotene, vitamin E was reported to be destroyed by UV light in skin [98, 99].

Weak Retinoid Activity is Generated from β-Carotene in HaCaT Cells and Reduced by UVA

β-Carotene served as a precursor for RA in HaCaT cells, although only to a minor degree, as demonstrated by the transactivation of an RA-dependent reporter gene. No retinol or retinyl esters were detected after β-carotene supplementation in HaCaT cells. This is consistent with the low expression level of the central β-carotene cleavage enzyme, β-carotene-15,15′-oxygenase. In addition, Torma et al. have shown defective retinol esterification in HaCaT cells [100]. Also, HaCaT cells are known to express the RA-degrading enzyme CYP26 at high levels [101]. Such a constellation should cause the low amounts of RA formed from β-carotene to be rapidly degraded, leaving trace amounts of RA for gene regulation. Eccentric cleavage products of β-carotene, apocarotenals, were present at detectable concentrations in HaCaT cells. Although apocarotenals can also be formed by oxidative breakdown, their prevalence is in accord with the higher expression of the eccentric cleavage enzyme β-carotene-9′,10′-oxygenase. Apocarotenals can be metabolized to RA via β-oxidation[102], and may well serve as the precursors for the RA that was indirectly detected by monitoring gene regulation. There is only scarce information available for the regulation of the two cloned β-carotene cleavage enzymes, β-carotene-15,15′-oxygenase[103, 104] and β-carotene-9′,10′-oxygenase. Both enzymes were not influenced by the treatments on the RNA level. β-Carotene-15,15′-oxygenase activity in duodenum, a tissue with high β-carotene cleaving activity, is suppressed by β-carotene, apo-8′-carotenal, retinol, or RA in rats [103]. Takeda et al. reported that β-carotene-15,15′-oxygenase activity was induced in skin of UV-irradiated SKH-1 hairless mice [105]. In HaCaT cells, this regulation is less obvious, most likely due to marginal RA production from β-carotene in HaCaT cells.

To differentiate the pro-retinoid effects of β-carotene in interaction with UVA from its ¹O₂ quenching, the gene expression profiles of RARs and RXRs, which are required to transduce the RA effects, were characterized. Moreover, RAR represents one of the best characterized RA target genes. Human epidermis expresses RARα, RARγ, RXRα, and RXRβ, as detected by Northern blot analysis [106]. Transcript levels for RAR reportedly are low or undetectable, and RXRγRNA was not detected. HaCaT cells were shown to express RARβ, in addition to RARα, RARγ, and RXRα[100]. Thus, HaCaT cells express all six retinoid receptor genes.

UVA downregulation of retinoid receptors is in line with reports from Wang et al. [107]. They showed that UV irradiation of human skin causes downregulation of RARγ and RXRα, which can be prevented by pretreatment with RA. β-Carotene had no significant effect on the expression levels of RARγ and RXRα, but non-significantly induced RAR in a dose-dependent manner. This result is consistent with low amounts of RA being formed from β-carotene, which suffice for a mild induction of the RA target gene RAR [108, 109], and for induction of the extremely sensitive artificial promoter of the reporter gene containing five RAREs. The RARs and RXRs other than RAR also contain autoregulatory elements in their promoters [110-112], but they are much less sensitive to induction by RA than RARβ.

In addition to activating RARE-dependent transcription, RA inhibits gene expression by transrepression of AP-1. Since MMP induction by UV light is mainly regulated by AP-2 and AP-1, RA would be expected to suppress UV-induced MMP expression. Indeed, Fisher and Voorhees have shown that UV(B) induction of MMPs 1, 3, and 9 in human skin can be prevented by RA pretreatment [70]. At the same time, DNA binding by AP-1 was reduced. The dose response curve for AP-1 transrepression by RA is not necessarily identical to transactivation of an RARE, or of a reporter construct driven by 5 RAREs. For two fibroblast cell lines collagenase expression is reduced by 10 nM RA [113, 114]. However, 1, 10, or 100 nM RA had no effect on UVA-induced MMP-1 secretion in this system (Goralczyk, unpublished observations). This rules out that downregulation of UVA-induced MMP-1 expression is mediated by β-carotene-derived retinoid activity.

Moreover, RA, and 1, 25-dihydroxyvitamin D3 (1,25-(OH)_{2D3}), may play a role in the regulation of the genes analyzed in this study. HaCaT cells were shown to synthesize 1,25-(OH)_{2D3} upon stimulation, e.g., with EGF [115]. Since the cellular response to UV involves an activation of the EGFR and downstream signalling pathways, UVA irradiation may well increase 1,25-(OH)_{2D3} synthesis in HaCaT cells.

In rheumatoid synovial fibroblasts, 1,25-(OH)_{2D3} inhibited IL1-induced MMP-1 and MMP-3 secretion[116]. If this is also the case in HaCaT cells, it would imply that MMP-1 and 3 induction by UVA would be even higher than if no 1,25-(OH)_{2D3} was synthesized upon UVA irradiation. If (all-trans)-β-carotene contributes to increased 9-cis RA formation, such an increased ligand concentration of both 1,25-(OH)_{2D3} and 9-cis RA could mediate a further decrease of MMP expression and thus contribute to the photoprotective effect of β-carotene. On the other hand, the microarray data show that UVA irradiation caused a downregulation of both VDR and RXRα(both downregulated by about 50%; [53]), indicating that the VDR system does not play a major role in this setting.

In sum, β-carotene suppressed UVA-induction of MMP-1, MMP-3, and MMP-10, which represent matrix metalloproteases crucially involved in degradation of the extracellular matrix during premature skin aging. Not only MMP-1, but also MMP-10 is regulated by ¹O₂-dependent pathways, and that β-carotene quenched ¹O₂-mediated induction of both MMP-1 and MMP-10. Vitamin E did not cooperate with β-carotene to further reduce UVA-induced MMP-1, MMP-3, or MMP-10 expression. HaCaT cells produced minute amounts of compounds with retinoid activity from β-carotene, as detected by marginal induction of RARβ and an RARE-dependent reporter gene. This feature renders HaCaT cells an excellent cell system to dissect and characterize the effect of the intact β-carotene molecule from the vitamin A activity of its metabolites.

### Example A

UVA exposure is thought to cause skin aging mainly by singlet oxygen (¹O₂)-dependent pathways. Using microarray hybridization the effect of pretreatment with the ¹O₂ quencher β-carotene (1.5 μM) on prevention of UVA-induced gene regulation in HaCaT human keratinocytes was explored.

### β-Carotene and UVA Treatment of Keratinocytes

The cell culture experiments were carried out as described [137]. Briefly, a subclone of passage 65 HaCaT keratinocytes, selected for differentiation capacity, was used at passages 16 to 23 after subcloning. 2×10⁵ cells were seeded per 60 millimeter dish. Starting the following day, the cells were pretreated for 2 days with β-carotene at 1.5 μM, a typical concentration in human plasma after moderate dietary supplementation [135])

β-carotene-containing medium was prepared as follows. Fresh all-E-β-carotene (DSM Nutritional Products, Kaiseraugst, Switzerland) stock solution in THF (containing 0.025% BHT; Fluka Chemie A G, Switzerland) was diluted 1:2 with ethanol and added to cell culture medium to a final concentration of 1.5 μM β-carotene. The solvent concentration in the medium was 0.5% for all treatments. β-carotene-containing medium was prepared fresh for the daily medium changes.

On day 3 of the experiment, the cells were irradiated with a Hönle sun lamp Sol 500 (270 kJ/m²; Dr. Hönle, Germany).

Cellular uptake of β-carotene from the culture medium was confirmed by HPLC analysis. Cells contained 20.06±5.66 μmol β-carotene/10⁶ cells after incubation with medium containing 1.85±0.09 μM β-carotene. During the 24 hours of incubation, the β-carotene concentration dropped to approximately 50% (not shown), irrespective of the presence of cells. No β-carotene was detected in placebo controls.

### Affymetrix GeneChip® Analysis

Five independent, factorially designed cell irradiation experiments were analyzed by microarray hybridization. For each experiment, one chip was hybridized per treatment condition. GeneChip® analysis was done as described in [132], which is incorporated by reference, as if recited in full herein. Gene regulation by β-carotene and/or UVA was calculated relative to placebo.

Gene regulation is reported as “change factors”, defined as “(treatment/control)−1” (in case of an increase), or “−(control/treatment)+1” (in case of a decrease), or zero (in case of no change). Changes in gene expression were included in further analysis only if the change factor was ≧0.5 or 0.5, and if unpaired t-tests yielded p values≦0.05. Upregulations by a change factor of 0.5 are labeled bold, downregulations by a change factor of 5-0.5 are labeled bold italics. (Table 1) To identify the pathways affected by the treatments functional information on the genes was retrieved from public literature databases.

It was determined that 1458 genes were significantly regulated by at least one of the treatments. β-carotene regulated 381 genes. UVA radiation influenced 568 genes. 1142 genes were regulated by co-treatment with UVA radiation and β-carotene. Of these, 610 were not regulated by treatments with only UVA radiation or β-carotene alone.

UVA irradiation produced downregulation of growth factor signalling, moderate induction of proinflammatory genes, upregulation of immediate early genes including apoptotic regulators, and suppression of cell cycle genes. Of the 568 UVA-regulated genes, β-carotene reduced the UVA-induced effect for 143 genes, enhanced it for 180 genes, and had no effect for 245 genes. The different interaction modes imply that β-carotene/UVA interaction involved multiple mechanisms.

In unirradiated keratinocytes, gene regulations suggest that β-carotene reduced stress signals and extracellular matrix (“ECM”) degradation, and promoted keratinocyte differentiation. In irradiated cells, expression profiles indicate that β-carotene inhibited UVA-induced ECM-degradation, and enhanced UVA induction of tanning-associated PAR-2. Combination of β-carotene-promoted keratinocyte differentiation with the cellular “UV response” caused synergistic induction of cell cycle arrest and apoptosis.

β-carotene at physiological concentrations interacted with UVA radiation effects in keratinocytes by mechanisms that included, but were not restricted to ¹O₂ quenching. The retinoid effect of β-carotene was minor, indicating that the β-carotene effects reported here were predominantly mediated through vitamin A-independent pathways.

TABLE 1
Transcriptional response to β-carotene and/or UVA treatment.

| | | | | |
|---|---|---|---|---|
| | | β- | UVA | UVA and β- |
| Acc. No. | Gene | Carotene | Radiation | Carotene |

Immediate Early Genes/Oxidative Defense

| | | | | |
|---|---|---|---|---|
| AB020315 | DKK-1; dickkopf-1 | −0.3 | 0.76 | 1.4 |
| U10550 | GEM | −2.6 | 0.91 | 3.91 |
| AB017642 | OSR1; oxidative-stress responsive 1 | −0.18 | 0.74 | 0.59 |
| U60207 | KRS-2; stress responsive serine/threonine | −0.8 | −0.7 | −0.35 |
| | protein kinase | | | |
| AL022312 | ATF4; activating transcription factor 4 | 0.11 | 0.76 | 0.96 |
| V01512 | C-FOS | −0.14 | 0.81 | 2.49 |
| V01512 | C-FOS | −0.16 | 0.36 | 1.36 |
| X16707 | FRA-1 | 0.14 | 0.61 | 0.71 |
| X16706 | FRA-2 | −0.7 | 0 | −0.13 |
| J04111 | C-JUN | −0.06 | −0.13 | 0.93 |
| M29039 | JUNB | −0.38 | −0.19 | −0.72 |
| X51345 | JUNB | −0.5 | −0.28 | −0.7 |
| X56681 | JUND | 0.6 | 1.65 | 3.2 |
| X56681 | JUND | 0.01 | 1.31 | 1.83 |
| X56681 | JUND | 0 | 1.17 | 1.66 |
| AL021977 | MAF-F | −0.26 | 1.49 | 1.71 |
| V00568 | c-myc | 0.1 | 0.37 | 3.22 |
| V00568 | c-myc | 0.1 | 0.34 | 2.45 |
| M55914 | c-myc binding protein (mbp-1) | 0.5 | 0 | 0.54 |
| U40992 | hsp40; heat shock protein 40 | −0.6 | −0.36 | −0.07 |
| M32011 | NCF2; p67-phox; neutrophil oxidase factor | −0.8 | 0.26 | −0.16 |
| AF020761 | stimulator of Fe transport | 0.03 | 0.71 | 1.03 |
| M13699 | ceruloplasmin (ferroxidase) | 1 | 0.58 | 1 |
| X01060 | transferrin receptor | −0.05 | 0.49 | 0.91 |
| L20941 | ferritin heavy chain | 0.36 | −0.03 | 0.59 |
| U60319 | haemochromatosis protein (hla-h) | 0.15 | −0.5 | −0.61 |
| Y00451 | 5-aminolevulinate synthase | 0.08 | 0.53 | 0.41 |
| D38537 | protoporphyrinogen oxidase | −0.32 | −0.34 | −0.54 |
| J03824 | uroporphyrinogen III synthase | −0.15 | −0.16 | −0.61 |
| M57951 | bilirubin udp-glucuronosyltransferase isozyme 2 | −0.25 | −0.15 | −0.66 |
| D16611 | coproporphyrinogen oxidase | −0.21 | −0.06 | −0.7 |
| L24123 | NRF1 | −0.27 | −0.08 | −0.65 |
| U13045 | NRF2, subunit beta 1 | −1.5 | −0.42 | −0.11 |
| X91247 | thioredoxin reductase | −0.08 | 0.53 | 1.48 |
| S62138 | GADD153 | −0.45 | 4.11 | 7.64 |
| Z50194 | TDAG51; PQ-rich protein; PHLDA1 | 0.27 | 2.3 | 6.19 |
| U83981 | GADD34 | −0.11 | 1.16 | 2.62 |
| AF001294 | TSSC3/IPL | −0.6 | 1.28 | 1.02 |
| AF035444 | TSSC3 | −0.44 | 0.51 | 0.7 |
| S81914 | IEX-1 | −0.08 | 1.65 | 0.91 |
| X78992 | ERF-2 | 0.31 | 0.54 | 0.99 |
| AF050110 | TIEG, EGRα | −0.8 | 0.3 | −0.11 |

Extracellular Matrix

| | | | | |
|---|---|---|---|---|
| X07820 | MMP10 | −1.3 | 3.59 | 2.2 |
| X05232 | MMP-3 | −0.46 | 1.23 | 0.93 |
| M13509 | MMP-1 | −1.61 | 0.06 | −0.22 |
| M93056 | serpinB1 | 0.7 | 0.1 | 0.57 |
| AJ228139 | Lekti | 0.48 | 0.27 | 0.75 |

Inflammation

| | | | | |
|---|---|---|---|---|
| U88879 | TLR3; toll-like receptor 3 | 0.6 | −1.4 | −0.91 |

VEGF-Related Ligands and Receptors

| | | | | |
|---|---|---|---|---|
| AF024710 | VEGF | 0.02 | 2.35 | 1.86 |
| AF022375 | VEGF | −0.5 | 1.1 | 0.78 |
| M63978 | VEGF | −0.48 | 0.9 | 1.09 |
| AF035121 | VEGFR2; VEGF receptor 2; KDR; FLK- | 0.6 | −0.18 | −0.28 |
| | 1kdr/flk-1 | | | |
| M36711 | AP-2α | −0.02 | 0.04 | −0.67 |

IFNα/β

| | | | | |
|---|---|---|---|---|
| M14660 | IFIT2; ISG-54K; (interferon stimulated gene) | 0.18 | 2.09 | 1.17 |
| M14660 | IFIT2; ISG-54K; (interferon stimulated gene) | −0.21 | 0.84 | 0.58 |
| AF026941 | IFIT4; IFI60; cig5; RIGG | −0.23 | 15.2 | 5.46 |
| L05072 | IRF-1; interferon regulatory factor 1 | 0.16 | 0.77 | 0.29 |
| U53831 | IRF-7b; interferon regulatory factor 7b | 0.16 | 0.61 | 0.49 |
| AJ225089 | TRIP14; ‘2-5’ oligoadenylate synthetase | −0.07 | 1.36 | 0.67 |
| M24594 | IFIT1; IFI56 | 0 | 0.19 | −0.61 |
| M24594 | IFIT1; IFI56 | 0.05 | 0.23 | −0.71 |
| M97935 | IRF-9; p48; ISGF3γ; Interferon-stimulated | −0.18 | −0.11 | −0.67 |
| | transcription factor 3γ | | | |

Interleukins

| | | | | |
|---|---|---|---|---|
| X04430 | IL6; Interleukin 6; IFNβα2a | 0.6 | 0.65 | 2.29 |
| D49950 | IL18; IGIF (IFNγ inducing factor) | −0.8 | 0.43 | −0.18 |
| X52560 | C/EBPβ; NF-IL6 | −0.28 | 0.96 | 0.83 |
| M83667 | C/EBPδ; NF-IL6-β | 0.04 | 0.55 | 0.31 |
| U20240 | C/EBPγ | −0.09 | 0.65 | 0.71 |
| S78771 | NF-κB subunit | −0.1 | 0.45 | 0.56 |
| X61498 | NF-κB subunit | −0.07 | 0.45 | 0.7 |
| S76638 | NFκB; p50 | −0.46 | 0.47 | 0.55 |

Proteinase-Activated Receptors

| | | | | |
|---|---|---|---|---|
| M62424 | PAR-1; thrombin receptor | −0.5 | 0.13 | −0.05 |
| D10923 | HM74; PAR1-related | 0.18 | −1 | −0.55 |
| AF055917 | PAR-4; protease-activated receptor 4 | −0.49 | −0.44 | −0.57 |
| U67058 | PAR-2; proteinase activated receptor-2 | 0.03 | 2.92 | 3.32 |
| U34038 | PAR-2; proteinase activated receptor-2 | −0.27 | 1.65 | 1.91 |
| U34038 | PAR-2; proteinase activated receptor-2 | −0.03 | 1.26 | 1.34 |

Prostaglandin Synthesis and Signalling

| | | | | |
|---|---|---|---|---|
| U04636 | COX-2, cyclooxygenase-2 | −1.2 | −0.18 | 0.2 |

EGF-Related Ligands and Receptors

| | | | | |
|---|---|---|---|---|
| M60278 | HB-EGF; heparin-binding egf-like growth | 0.33 | 1.53 | 3.32 |
| | factor | | | |
| X00588 | EGFR; precursor of epidermal growth factor | −0.6 | −0.34 | −0.69 |
| | receptor | | | |
| H06628 | ERBB3 precursor; similar to | 0 | −0.07 | −0.78 |
| M34309 | HER3; epidermal growth factor receptor | −0.28 | 0.04 | −1.12 |
| | (her3) | | | |
| M34309 | HER3; epidermal growth factor receptor | −0.6 | −0.04 | −1.56 |
| | (her3) | | | |

FGF-Related Ligands and Receptors

| | | | | |
|---|---|---|---|---|
| M27968 | bFGF; basic fibroblast growth factor; FGF2 | 0.1 | 0.91 | 0.82 |
| M87770 | FGFR2; FGF receptor 2 | 0.08 | −0.6 | −0.48 |
| M64347 | FGFR3; FGF receptor | −1.1 | −0.2 | −2.58 |

TGFβ-Related Ligands and Receptors

| | | | | |
|---|---|---|---|---|
| X02812 | TGFβ; transforming growth factor β | 0.9 | 0.32 | 0.46 |
| M22489 | BMP2a; bone morphogenetic protein 2a | −1.2 | 0.1 | 0.16 |
| | (bmp-2a) | | | |
| M62302 | GDF-1; growth/differentiation factor 1 (gdf-1) | −0.8 | −0.14 | −0.43 |
| U59423 | SMAD 1 | −0.27 | −0.34 | −0.57 |
| U68019 | SMAD3 | 0.5 | −0.29 | 0.01 |
| U68019 | SMAD3 | 0.18 | −0.6 | −0.09 |
| U44378 | SMAD4 | −0.7 | 0.06 | −0.67 |
| U59913 | SMAD5 | −0.9 | −0.3 | −0.84 |
| AF035528 | SMAD6 | −0.47 | −7.3 | −24.2 |
| AF010193 | SMAD7 | −0.19 | −0.27 | −0.63 |

WNT Signalling

| | | | | |
|---|---|---|---|---|
| I20861 | WNT5A | −0.47 | −0.7 | −2.22 |
| I20861 | WNT5A | −1.7 | −1.5 | −3.34 |
| I37882 | frizzled-2 | −0.03 | −0.27 | −1.38 |
| AB012911 | frizzled-6 | −0.5 | −0.7 | −1.16 |

IGF/Insulin Signalling

| | | | | |
|---|---|---|---|---|
| M35878 | IGF-BP 3; insulin-like growth factor-binding | 0.29 | 1.95 | 1.64 |
| | protein-3 gene | | | |
| M35878 | IGF-BP 3; insulin-like growth factor-binding | 0.22 | 1.81 | 1.73 |
| | protein-3 gene | | | |
| X96584 | NOV | −0.7 | 2.43 | 0.72 |

Jagged/Delta Signalling

| | | | | |
|---|---|---|---|---|
| AF029778 | jagged2 (jag2) | −0.02 | −0.6 | −0.67 |
| U97669 | NOTCH3 | −0.8 | −0.42 | −1.37 |

MAPK Pathway

| | | | | |
|---|---|---|---|---|
| M54968 | K-RAS | −0.6 | −0.23 | −0.64 |
| X02751 | N-RAS | −1.2 | 0.1 | −0.01 |
| D87116 | MAPKK3b; MKK3b | −0.06 | 0.38 | 0.62 |
| L35263 | MAPK14; p38; csaids binding protein (csbp1) | −0.38 | −0.25 | −0.53 |
| U09759 | MAPK9; JNK2 | −0.36 | −0.08 | −0.57 |
| U71087 | MAPKK MEK5b | −0.16 | −0.42 | −0.7 |
| D45906 | LIM kinase 2 (limk-2) | −0.3 | −0.02 | −0.66 |
| U43195 | p160ROCK | −0.28 | −0.31 | −0.55 |
| U67156 | MAPKKK5; ASK1 | −0.06 | −1.8 | −4.7 |
| U48807 | MAP kinase phosphatase (mkp-2) | 0 | 1.1 | 1.12 |
| U15932 | DUSP5 | −0.15 | 1.87 | 2.84 |
| X93921 | DUSP7 | −0.46 | 1.13 | 0.99 |

Differentiation Markers

| | | | | |
|---|---|---|---|---|
| AF019084 | keratin 2e (KRT2E); Keratin 2A | −0.46 | −0.17 | −0.69 |
| M21389 | keratin 5 | 0.9 | 0.11 | 0.81 |
| J00124 | keratin 15 | 1 | −0.11 | 0.96 |
| M28439 | keratin 16 | −2 | 0.08 | −0.38 |
| Z19574 | keratin 17 | 0.04 | 0.03 | 0.6 |
| M69225 | BPAG1; bullous pemphigoid antigen | −0.5 | 0.91 | 0.12 |
| M91669 | bullous pemphigoid autoantigen bp180 | −0.45 | 0.07 | −0.57 |
| X56807 | DSC2; desmocollin type 2a and 2b | −1.1 | 0.35 | −0.29 |
| D17427 | desmocollin type 4 | −1.3 | −0.19 | −1.71 |
| X53586 | integrin α6 | −1.1 | 0 | −0.76 |
| S66213 | integrin α6b | −0.9 | 0.04 | −0.46 |
| S66213 | integrin α6b | −1.3 | −0.16 | −0.73 |
| U40282 | ILK; integrin-linked kinase | −0.23 | −0.13 | −0.51 |
| AF099730 | connexin 31 | 0.17 | 1.91 | 2.3 |
| U03493 | connexin 45 | −0.06 | 0.68 | 0.26 |
| X05610 | collagen type IV, α-2 (COL4A2) | −0.13 | −0.6 | −0.98 |
| M58526 | collagen type IV, α-5 (COL4A5) | −0.8 | −1.1 | −1.16 |
| D21337 | collagen type IV, α-6 (COL4A6) | −0.44 | −0.18 | −0.71 |
| L02870 | collagen type VII, α-1 (COL7A1) | −0.21 | −0.15 | −0.83 |
| U70663 | KLF4; EZF (epithelial Zn finger) | −0.17 | 1.99 | 3.46 |

Cell Cycle
G1 Phase

| | | | | |
|---|---|---|---|---|
| M73812 | cyclin E | −0.23 | 1.6 | 1.75 |
| AF091433 | cyclin E2 | −0.25 | 0.89 | 0.26 |
| M33764 | ornithine decarboxylase | −0.2 | 1.25 | 0.57 |
| X16277 | ornithine decarboxylase | −0.32 | 0.73 | 0.37 |
| X77743 | CDK activating kinase | −0.01 | 0.4 | 0.57 |
| U22398 | CDK-inhibitor p57KIP2 (KIP2) mrna | −0.22 | 1.25 | 0.79 |
| U03106 | p21; wild-type p53 activated fragment-1 | −0.5 | 0.26 | 0.21 |
| | (WAF1) | | | |
| L25876 | CIP2; CDKN3 | −0.18 | −0.47 | −0.81 |
| X55504 | NOL1; p120 nucleolar antigen | 0.04 | 0.52 | 0.94 |
| AB024401 | p33; ING1b | −0.47 | 0.66 | 0.64 |
| L49229 | RB1 | −0.8 | −0.8 | −1.13 |
| X74594 | RB2/p130 | −0.6 | −0.6 | −0.85 |
| AL021154 | ID3; HEIR1 | −0.43 | −2.2 | −4.35 |
| X77956 | ID1 | −0.01 | −1.6 | −2.43 |
| X77956 | ID1 | −0.11 | −3.6 | −4.7 |
| D13891 | ID-2H | −0.7 | −1 | −2.68 |
| AL022726 | ID-4 | −0.08 | −7.2 | −0.49 |

S Phase: DNA Integrity Checkpoint, DNA Replication and Repair

| | | | | |
|---|---|---|---|---|
| L20046 | ERCC5; excision repair protein | −0.48 | −0.42 | −0.95 |
| U47077 | DNA-PK, catalytic subunit | −0.7 | −0.32 | −1.06 |
| M30938 | KU (p70/p80) | −0.18 | −0.28 | −0.64 |
| U40622 | XRCC4 | −0.05 | −0.9 | −0.01 |
| X65550 | mKI67a mrna (long type) for antigen of | −0.3 | −0.8 | −0.87 |
| | monoclonal antibody KI-67 | | | |
| X65550 | mKI67a mrna (long type) for antigen of | −0.17 | −1 | −1.59 |
| | monoclonal antibody KI-67 | | | |
| X67098 | rTS α | 0.13 | −0.7 | −0.47 |
| X02308 | thymidylate synthase | −0.12 | −0.23 | −0.56 |
| X84740 | DNA ligase III | −0.5 | −0.19 | −0.71 |
| X06745 | DNA polymerase α-subunit | −0.43 | −0.36 | −0.63 |
| X74331 | DNA primase (subunit p58) | −0.16 | −0.5 | −0.41 |
| L07493 | RPA; replication protein A 14 kda subunit | −0.04 | −0.08 | −0.52 |
| | (rpa) | | | |
| L47276 | α topoisomerase truncated-form | −0.43 | −1 | −0.81 |
| J04088 | TOP2; topoisomerase II | −0.22 | −0.7 | −0.86 |

G2/M Phase

| | | | | |
|---|---|---|---|---|
| U14518 | CENP-A; centromere protein-A | −0.13 | −0.7 | −1.82 |
| Z15005 | CENP-E; centromere protein-E | −0.37 | −1.4 | −1.44 |
| U30872 | CENP-F; mitosin | −0.42 | −1.2 | −1.22 |
| AF083322 | CEP110; centriole associated protein | 0.15 | −0.6 | −4.18 |
| AF011468 | STK15; BTAK | −0.18 | −0.7 | −1.85 |
| X62048 | WEE1 | −0.6 | 0.38 | 0.05 |
| AF053305 | BUB1; mitotic checkpoint kinase | −0.5 | −0.8 | −0.81 |
| AF053306 | MAD3L; mitotic checkpoint kinase | −0.18 | −1 | −2.45 |
| U37426 | KINESIN_LIKE 1; KNSL1; HKSP; EG5 | −0.04 | −0.6 | −0.92 |
| D14678 | KINESIN-LIKE 2; HSET | −0.04 | −0.3 | −0.72 |
| D14678 | KINESIN-LIKE 2; HSET | 0.03 | −0.42 | −1.15 |
| AL021366 | KINESIN-LIKE 2; HSET | −0.34 | −0.7 | −0.72 |
| X67155 | KINESIN-LIKE 5; KNSL5; MKLP-1; mitotic | −0.15 | −0.7 | −1.04 |
| | kinesin-like protein-1 | | | |
| U63743 | KINESIN-LIKE 6; KNSL6; MCAK; mitotic | −0.15 | −0.8 | −1.61 |
| | centromere-associated kinesin | | | |

Apoptosis

| | | | | |
|---|---|---|---|---|
| U19599 | BAXδ | 0.6 | −0.23 | 0.67 |
| L22475 | BAXγ | 0.8 | 0.17 | −0.34 |
| AB020735 | ENDOGL-2 | 0.8 | 0.35 | 0.41 |
| D90070 | NOXA | −0.26 | 0.85 | 0.74 |
| U67319 | caspase 7 | −0.09 | 0.62 | 0.31 |
| M96954 | TIAR; nucleolysin tiar | −0.45 | −0.7 | −0.08 |
| U13022 | caspase 2, ICH-1S | −0.31 | −0.23 | −0.62 |
| AF001433 | Requiem | 0.5 | 0.15 | 0.32 |
| U83857 | AAC11 | −0.6 | −0.19 | −0.05 |
| U37518 | TRAIL; TNF-related apoptosis inducing ligand | 0.15 | −3 | −2.66 |
| U77845 | TRIP | −0.06 | 0.04 | −1.35 |
| U84388 | CRADD; death domain containing protein | −0.25 | −0.46 | −0.87 |
| L41690 | TRADD; TNF receptor-1 associated protein | 0.23 | −0.19 | −0.85 |
| U79115 | RAIDD; death adaptor molecule | −0.22 | −0.4 | −0.66 |
| AF005775 | CLARP; CFLAR, alternatively spliced | −0.09 | −0.44 | −0.62 |

RA Targets

| | | | | |
|---|---|---|---|---|
| AF061741 | RETSDR1; retinal short-chain dehydrogenase/ | 1.1 | −0.9 | −0.06 |
| | reductase | | | |
| AJj005814 | HOXA7 | −0.43 | −0.31 | −0.52 |
| S82986 | HOXC6 | −0.07 | −0.11 | −0.82 |
| X59373 | HOXD4 | −0.5 | −0.8 | −0.76 |
| AF017418 | MEIS2 | 0.26 | −1.4 | −1.3 |
| M64497 | COUP-TF II; ARP1; apoA1 regulatory protein | 0.33 | 0.27 | 0.81 |
| U37146 | SMRT | −0.33 | −0.5 | −1.22 |
| X52773 | RXRα | −0.2 | −0.5 | −0.57 |
| U66306 | RXRα | −0.2 | −0.43 | −0.61 |

### A) β-Carotene Effects in Unirradiated Keratinocytes:

β-Carotene Reduced Stress Responses

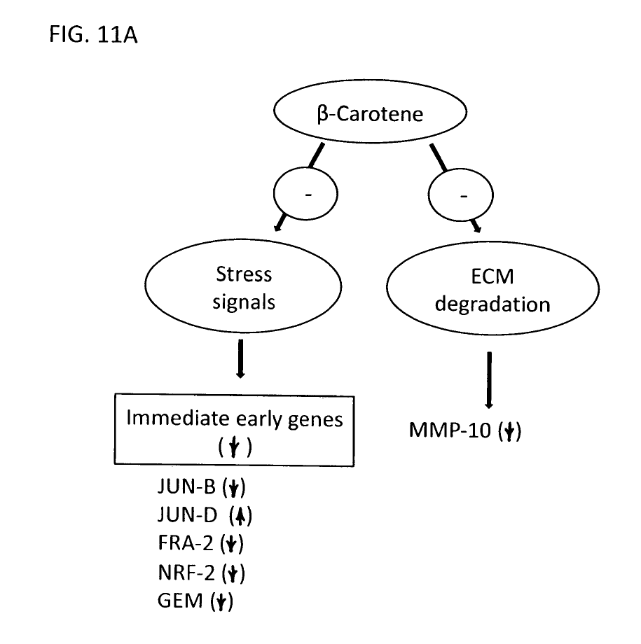

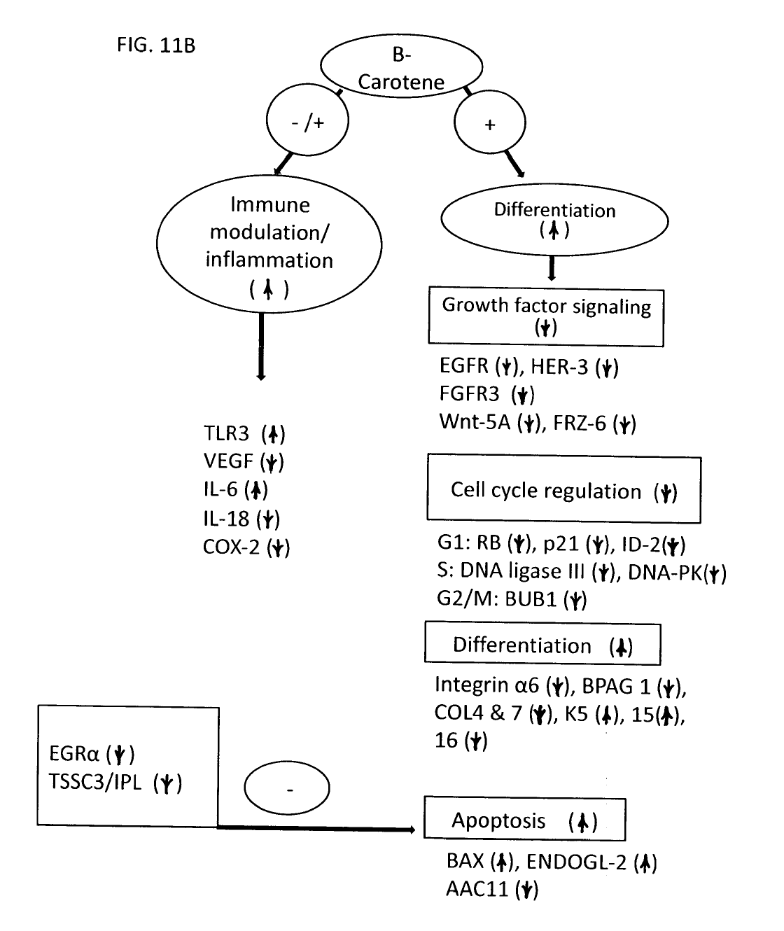

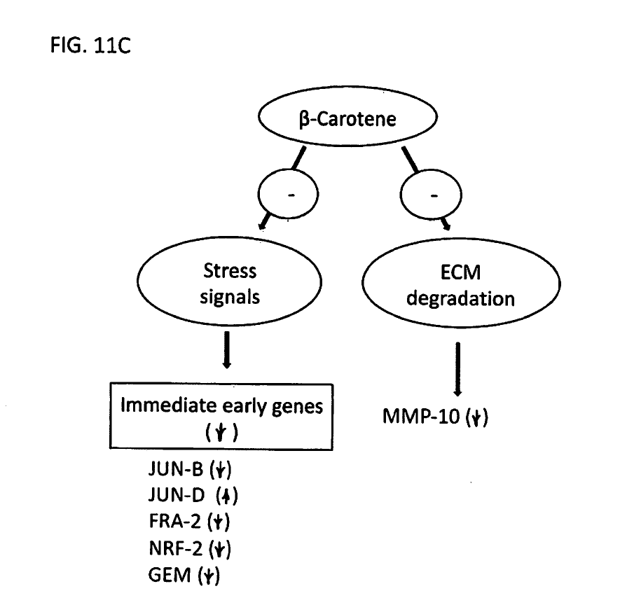

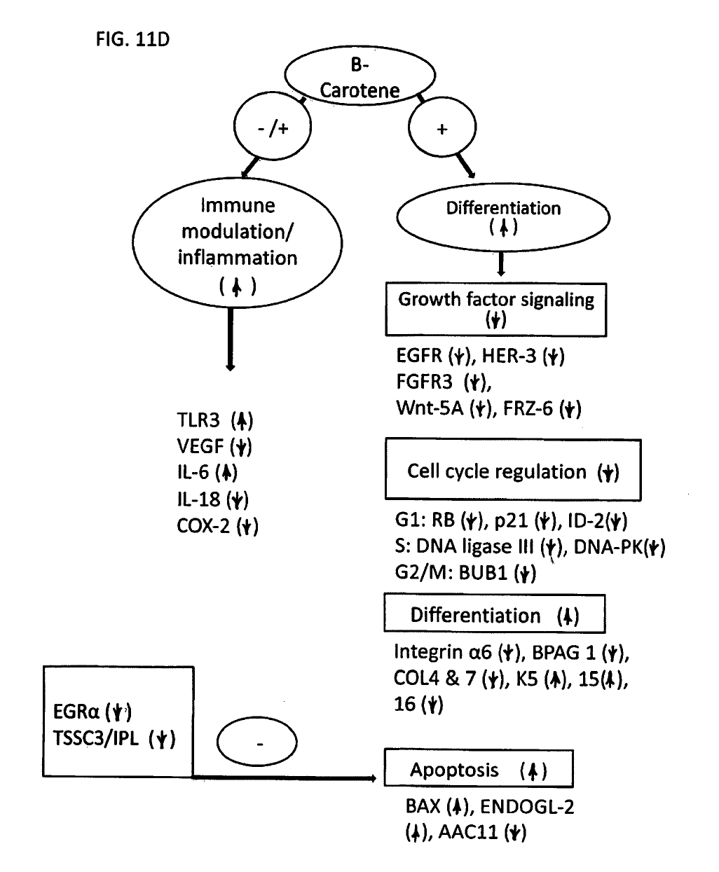

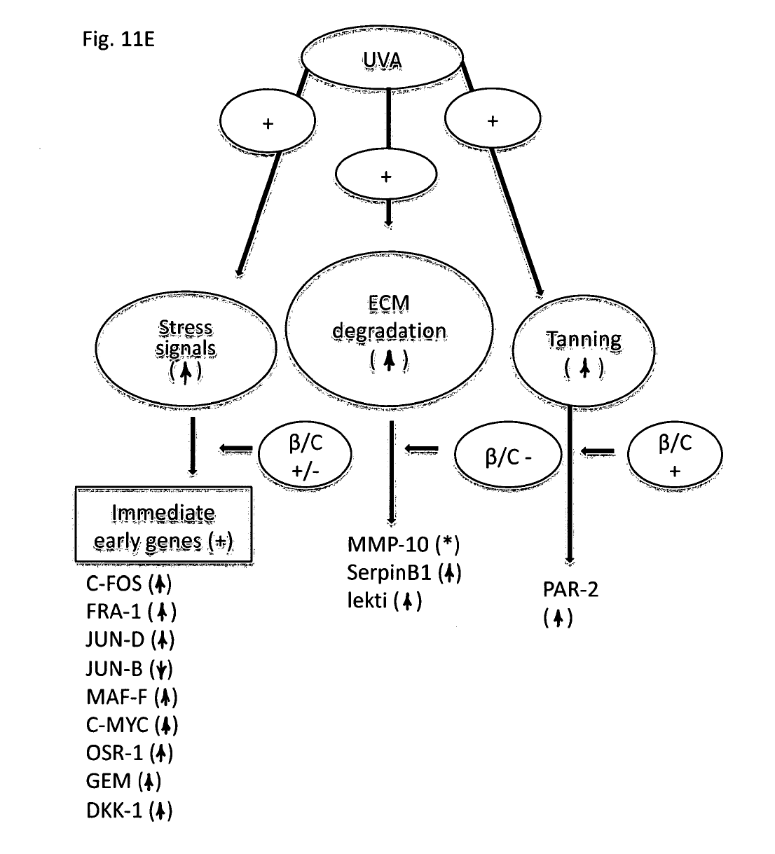

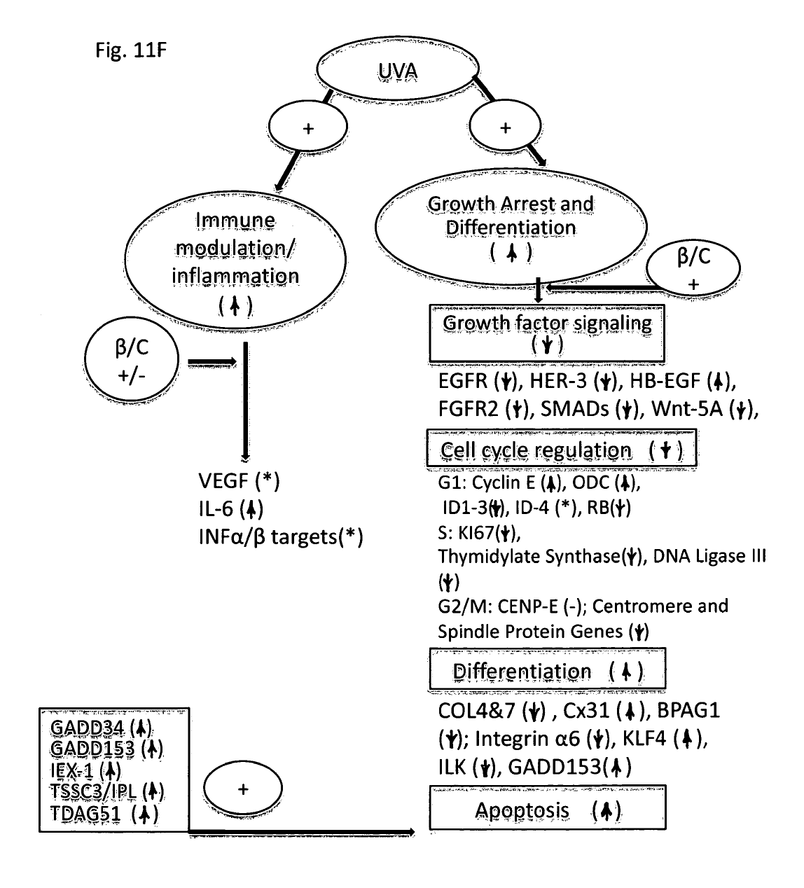

Stress stimuli, like UV irradiation or oxidative stress, e.g., resulting from ROS production in the respiratory chain, elicit a cellular stress response, leading to the induction of immediate early genes. (β-carotene downregulated several immediate early genes (GEM, KRS-2, JUN-8, FRA-2, EGRa) and oxidative stress defense genes (NCF2, NRF2β1). This suggests that β-carotene reduced cellular stress including oxidative stress in unirradiated keratinocytes. (FIGS. 11 a and 11 b)

### β-Carotene Reduced Basal MMP-10 Expression

Degradation of ECM molecules by matrix metalloproteases (MMPs) in skin is a key process in skin aging. β-carotene reduced the basal expression of MMP-10. This was confirmed by QRT-PCR in independent experiments [137). MMP-10 cleaves various ECM molecules, but also activates other MMPs. Due to its broad substrate specificity, MMP-10 is likely involved in MMP-mediated skin aging.

Together with the finding that (R-carotene mildly reduces basal MMP-1 expression[137], this indicated that β-carotene reduces ECM degradation in unirradiated skin, and can therefore delay skin aging.

### β-Carotene Promoted Normal Keratinocyte Differentiation

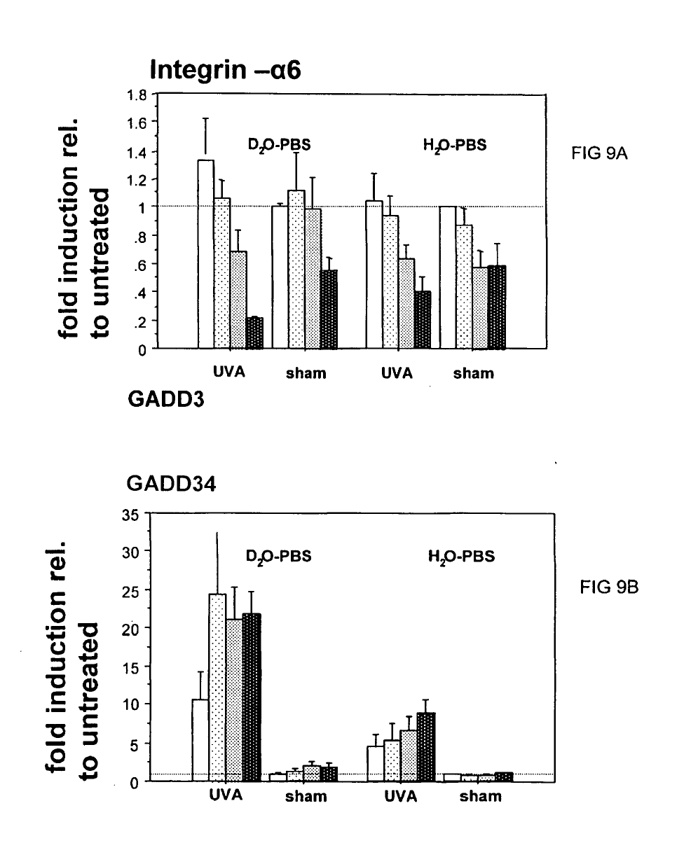

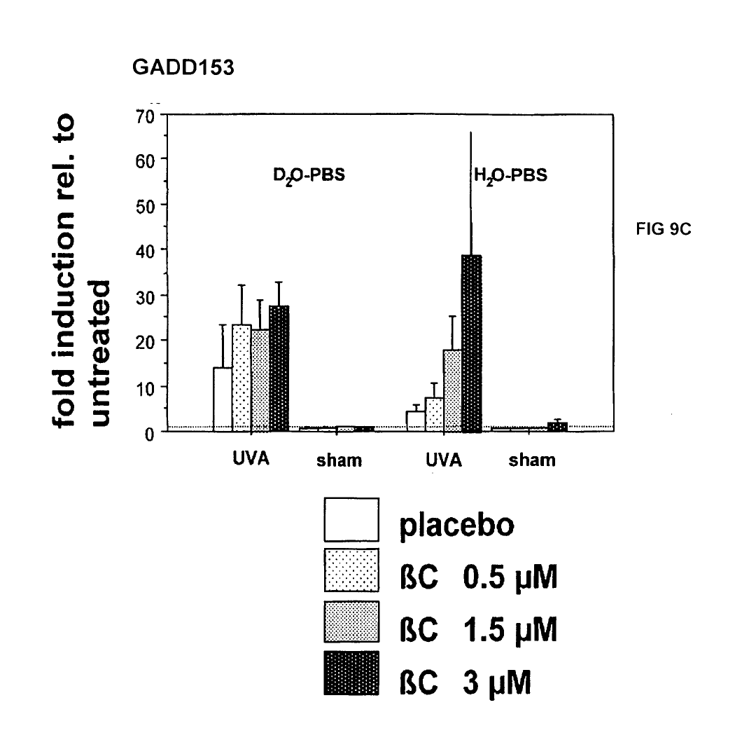

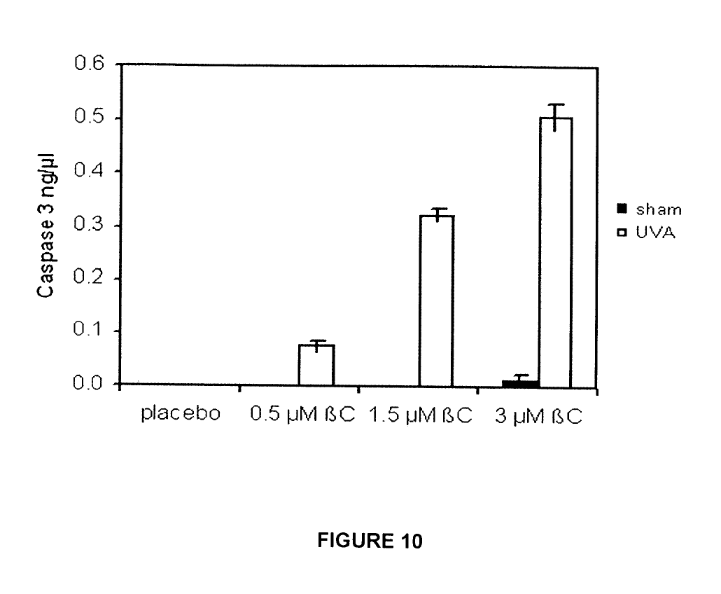

The response of HaCaT cells to β-carotene treatment was consistent with the cells undergoing differentiation. First, β-carotene downregulated genes associated with growth factor signaling (e.g., EGFR, NOTCH3, BMP2a, and Wnt5a) and cell cycle regulation (e.g., ID-2, DNA ligase III, and BUB1). Second, β-carotene regulated marker genes for physiological keratinocyte differentiation. Keratin 15 transcription was decreased and transcription of basement membrane collagen COL4A5 and the hem idesmosomal cell adhesion molecules BPAG1 and integrin_{α6} was decreased. QRT-PCR confirmed downregulation of integrin_{α6} (FIG. 9 a). Since keratinocyte differentiation involves apoptosis, it is interesting that β-carotene upregulated several proapoptotic genes (Bax, endogl-2, requiem). This was counterbalanced, in part, by downregulation of immediate early genes, some of which favor apoptosis (e.g., TSSC3/IPL, EGRa). Apparently, β-carotene treatment prepared cells for apoptosis, but was not sufficient to induce apoptosis, as confirmed in a functional apoptosis assay (FIG. 10; unirradiated cells). This indicated that β-carotene promoted differentiation, but did not induce terminal differentiation in keratinocytes.

### β-Carotene Differentially Regulated Immune Modulators

β-carotene reportedly stimulates immune function [127]. β-carotene upregulated TLR3, a receptor involved in innate immunity, and IL-6, an important regulator of inflammation, keratinocyte growth, and wound healing. β-carotene mildly downregulated VEGF, a key angiogenic factor, and COX-2, the rate-limiting enzyme in prostaglandin synthesis. Moreover, β-carotene downregulated IL-18, an IL-12-related growth and differentiation factor for Th1 cells. Overall, β-carotene differentially regulated inflammatory signals in unirradiated keratinocytes.

### β-Carotene Acted Predominantly Via RA-Independent Pathways

Among presumed RA-regulated genes, only retinol short chain dehydrogenase 1 (retSDR1) was induced by β-carotene. Other known RA targets [117] were either not altered by β-carotene, or were downregulated (e.g., HOXD4), indicating that the effects of β-carotene described here were mainly RA-independent.

### B) β-Carotene Effects in UVA-Irradiated Keratinocytes

β-Carotene Interacts with UVA by Multiple Mechanisms

UVA irradiation elicited downregulation of growth factor-dependent signalling cascades, moderate induction of proinflammatory genes, induction of immediate early genes including apoptotic regulators, and suppression of cell cycle genes (FIGS. 11 c and 11 d). He et al. [126] made very similar observations in UVA-irradiated HaCaT cells. Of the 568 UVA-regulated genes, β-carotene quenched the UVA effect on 143 genes, i.e. they had expression profiles expected for ¹O₂-induced genes. On the other hand, β-carotene enhanced the UVA effect for 180 genes and had no influence on UVA regulation of 245 genes. These different modes of interference imply several mechanisms of UVA/R-carotene interaction.

### β-Carotene Inhibited Expression of MMP-10 and Promoted Expression of Protease Inhibitors

Chronic sun exposure causes degradation of ECM proteins by inducing MMPs in skin, leading to premature skin aging. In our experiments, UVA irradiation induced MMP-10. β-carotene inhibited MMP-10 expression in UVA-irradiated keratinocytes. MMP-10 induction involves ¹O₂, and β-carotene dose-dependently inhibited MMP-10 induction by UVA/D₂O. Hence, β-carotene acts as a ¹O₂ quencher in living cells. β-carotene also reduced the basal and ¹O₂-induced expression of MMP-1 and downregulated UVA induction of MMP-3 [137]. Furthermore, β-carotene upregulated the protease inhibitors Lekti and serpinB1. TIMP-1, a likely MMP-10 inhibitor, was not influenced by the treatments.

Overall, the data indicated that β-carotene diminished UVA-induced ECM degradation, indicating that β-carotene at physiological concentrations may delay photoaging. Green and coworkers provided preliminary clinical evidence that & carotene supplementation may indeed reduce wrinkling. (D. Battistutta, G. M. Williams and A. G. Green: Effectiveness of daily sunscreen application and β-carotene intake for prevention of photoaging: a community-based randomised trial. International Congress on Photobiology; 28th Annual American Society for Photobiology Meeting, 2000, San Francisco).

### β-Carotene Differentially Regulated Proinflammatory Genes

The cellular UV response includes induction of proinflammatory cytokines, but also immune suppression. β-carotene prevents UV-induced immune suppression [120] and alleviates erythema after sun exposure [123, 134].

UVA induced mild signs of inflammation. β-carotene reduced UVA upregulation of VEGF and IFNα/β targets. VEGF induction by UVA relies on an AP-2 site in the VEGF promoter [122], suggesting a ¹O₂-dependent regulation. VEGF downregulation may explain how β-carotene reduces erythema formation after sun exposure. IL-6 expression was weakly upregulated by UVA and enhanced by β-carotene. IL-6 is induced by IL-1 via a ¹O₂-dependent positive autoregulatory loop [15]. IL-6 can also be induced by SAPK/JNK signaling [83]. As β-carotene did not quench the UVA induction of JNK/SAPK target genes, it appears that increased IL-6 induction by UVA and β-carotene occurred through JNK/SAPK signaling instead of the ¹O₂-dependent loop. IL-6 induction is expected to counteract the β-carotene-mediated VEGF reduction, thus impeding a stronger protection against erythema by β-carotene.

### β-Carotene Enhanced UVA Induction Of PAR-2

PAR-2, a receptor required for tanning, was expectedly induced by UVA and further increased by β-carotene. Tronnier et al. [136] report that carotenodermia positively influences pigmentation disorders independent of tanning. Raab, et al. [131] and Postaire, et al. [130], however, found an increased melanin content in skin after supplementation with β-carotene-containing antioxidant mixtures. β-carotene enhanced UVA induction of PAR-2 explains how carotenoid supplementation increases tanning after sun exposure.

### β-Carotene Acted Predominantly Via RA-Independent Pathways

UVA depletes cellular retinol stores [133], possibly leading to reduced RA availability. Accordingly, RA target genes [117] were downregulated by UVA irradiation. Except for retSDR1, β-carotene did not restore expression of RA target genes. HaCaT cells produce low amounts of retinoid activity from β-carotene [137], rendering HaCaT cells an excellent model to evaluate provitamin A-independent functions of β-carotene.

### β-Carotene Further Promoted Differentiation in Irradiated Keratinocytes

Expression of differentiation markers indicated that β-carotene promoted keratinocyte differentiation more strongly in UVA-irradiated cells than in unirradiated cells. UVA/β-carotene treatment downregulated more genes encoding basement membrane collagens than did the single treatments. Downregulation of BPAG1, integrin_{α6}, ILK, desmocollins, and Cx45, as well as upregulation of Cx31, KLF4 and GADD153 also indicate keratinocyte differentiation. This effect may render combined β-carotene/UVA treatment a promising therapy for skin disorders associated with disturbed differentiation, e.g., psoriasis.

### β-Carotene Did not Prevent UVA-Induced Stress Signals

Activation of JNK/SAPK, NFκB, and induction of their target genes are hallmarks of the cellular UV response. Massive transcriptional counterregulation of these signaling pathways occurred upon UVA irradiation. Expression profiles of protein kinases and phosphatases, and upregulation of target genes (C-FOS, FRA-1, JUND, ATF4, MAF-F, DKK-1, GEM) are consistent with a stress response induced by SAPK/JNK activation. β-carotene did not inhibit these UVA effects and enhanced some.

Few genes associated with oxidative stress were regulated. UVA induced, e.g., OSR-1/STK25, a ROS-activated kinase, and thioredoxin reductase, which together with thioredoxin (Trx) acts at the core of antioxidant defense. β-carotene favored these protective gene regulations.

Overall the data suggest that stress signalling was activated by UVA. β-carotene did not inhibit these UVA effects, and enhanced some.

### “UV Response” of Keratinocytes Undergoing β-Carotene-Induced Differentiation LED to Cell Cycle Arrest and Apoptosis

SAPK/JNK signaling often leads to cell cycle arrest and apoptosis. Expression profiles of cell cycle regulators indicated that cell cycle arrest was induced by UVA and further enhanced by β-carotene.

UVA induced several genes which function during the G₁ cell cycle phase (cyclin E, p57^{KIP2}, ornithine decarboxylase). The vast majority of cell cycle regulators functioning in later cell cycle phases were downregulated by UVA, indicating cell cycle arrest at the late G₁ phase. Examples include the proliferation marker Ki67 and genes involved in DNA replication or encoding mitotic spindle proteins. Moreover, UVA downregulated several growth factor receptors and members of the downstream signalling machinery. β-carotene alone also downregulated genes involved in growth factor signalling, and reduced expression of cell cycle regulators in the context of its differentiation-promoting activity. Combined UVA/β-carotene treatment led to a more pronounced cell cycle arrest than did the single treatments.

Following cell cycle arrest, cells can re-enter the cell cycle or undergo apoptosis. Here, UVA irradiation induced several apoptotic regulators, including the immediate early genes IEX-1, GADD34, GADD153, ERF-2, and TSSC3/IPL. β-carotene enhanced UVA induction of GADD153, GADD34, TDAG51 and ERF-2. The expression profiles of GADD153 and GADD34 were confirmed by QRT-PCR (FIGS. 1 b and 1 c). The data are consistent with previous evidence that UVA causes apoptosis subsequent to SAPK/JNK activation (see also He, 2004). β-carotene did not reduce this UVA effect. Some gene regulation was enhanced by β-carotene.

Apoptosis induction was confirmed by assessing caspase-3 activity. Caspase-3 activity 5 hours after UVA irradiation was quantified in five separate experiments using the CaspACE™ Assay System (Promega/Catalys, Switzerland). Neither UVA nor β-carotene alone activated caspase-3. β-carotene cooperated with UVA to induce caspase-3 activity in a dose-dependent manner (FIG. 2).

Together, cells pretreated with β-carotene and irradiated with UVA underwent G₁ cell cycle arrest and apoptosis. If this process takes place in vivo β-carotene should favor sun burn cell formation. However, while a mild reduction in sunburn erythema was found in several studies, β-carotene supplementation did not alter the number of sunburn cells in humans [121]. Induction of apoptosis in the p53-deficient HaCaT cells would imply a favorable removal of precancerous cells, and β-carotene supplementation in most cases indeed reduced skin carcinogenesis in rodents (e.g., [129]). Clinical intervention trials, however, have found no significant prevention of non-melanoma skin cancer [125], [124] by β-carotene. Besides carotenoids, the skin contains other antioxidants, which are believed to prevent β-carotene from enhancing some of the UVA effects in vivo. Furthermore, HaCaT cells are exceptionally sensitive to UV-induced apoptosis [118]. Thus, even though the consequences in skin might be less pronounced than in HaCaT cells, it is possible that the mechanisms identified here nevertheless apply in vivo.

### Relationship of the Modes of Action of β-Carotene to its Influence on UVA-Induced Biological Processes

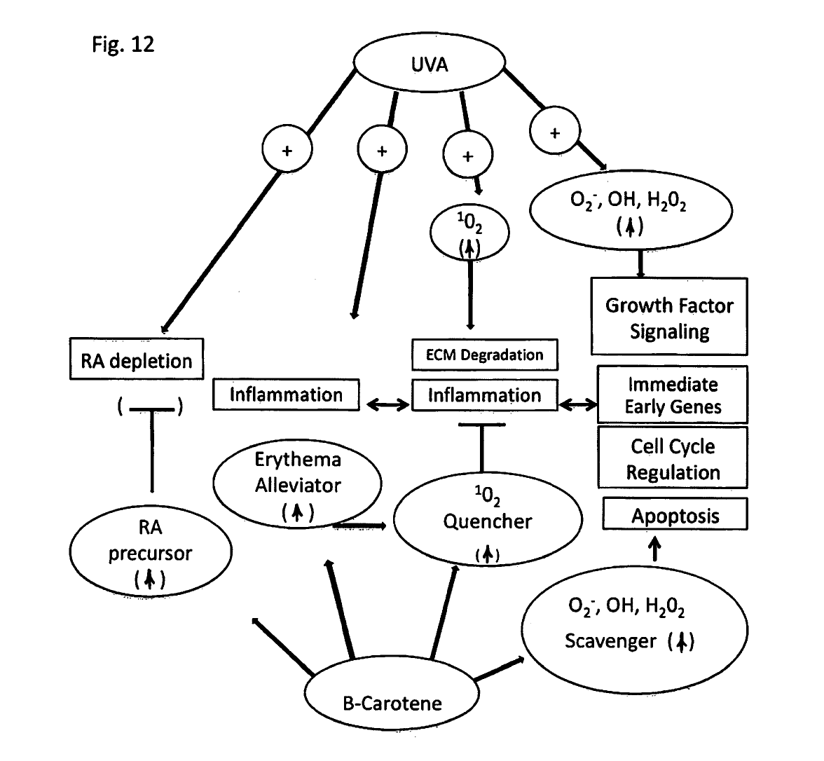

FIG. 12 shows the relationship of the modes of action of β-carotene to its influence on UVA-induced biological processes deduced from the experiments below. β-carotene reduced UVA-induction of genes involved in ECM degradation and inflammation as a ¹O₂ quencher. The mild photoprotective effect of β-carotene appears to be based on inhibition of these ¹O₂-induced gene regulations, rather than on a physical filter effect. A physical filter effect would be expected to reduce all UVA responses by the same amount. β-carotene, if scavenging ROS other than ¹O₂, is irreversibly damaged and converted into radicals, if not rescued by other antioxidants (Edge, 2000). Consistent with this observation, β-carotene did not inhibit UVA-induced stress signals and enhanced some. UVA exposure suppressed several RA target genes. Since HaCaT cells produce marginal amounts of retinoid activity from β-carotene, the provitamin A activity of β-carotene did not translate into restored expression of RA target genes in this system.

β-carotene at physiological concentrations interacted with UVA effects in keratinocytes by multiple mechanisms that included, but were not restricted to ¹O₂ quenching.

In unirradiated keratinocytes, β-carotene reduced expression of immediate early genes, indicating reduced stress signals. Moreover, gene regulation by β-carotene suggested decreased ECM degradation and increased keratinocyte differentiation. This effect on differentiation was unrelated to UVA exposure, but synergized with UVA effects.

In UVA-irradiated cells, β-carotene inhibited gene regulation by UVA, which promoted ECM degradation, indicating a photoprotective effect for β-carotene. β-carotene enhanced UVA-induced PAR-2 expression, suggesting that β-carotene enhanced tanning after UVA exposure. The combination of β-carotene-induced differentiation with the cellular “UV response” led to a synergistic induction of cell cycle arrest and apoptosis by UVA and β-carotene.

The retinoid effect of β-carotene was minor, indicating that the β-carotene effects reported here were predominantly mediated through vitamin A-independent pathways.

The results explain and integrate many conflicting reports on the efficacy of β-carotene as a ¹O₂ quencher and as a general antioxidant in living cells. The mechanisms identified, by which β-carotene acts on the skin, have implications on skin photoaging, as well as on relevant skin diseases, such as skin cancer and psoriasis.

### Example B Quantitative Real Time-Polymerase Chain Reaction

Key gene regulation was confirmed in three independent cell irradiation experiments using TaqMan® QRT-PCR as described [137]. The sequences of the primers and probes used are given in Table 2. In these experiments, cells were pretreated with 0.5, 1.5, or 3 μM β-carotene, to analyze for dose-dependent β-carotene effects. In addition, cells were irradiated either in D₂O-containing PBS or in H₂O-containing PBS, to analyze for the ¹O₂ inducibility of genes.

TABLE 2
Primers and probes used for QRT-PCR.

| Tran- | Forward | Reverse | |
|---|---|---|---|
| script | Primer | Primer | Probe |
| Integrin_{α5} | TTTCCCGTTTCT | TGGAAAAGGTAA | AGACTCCGTTAG |
| | TTCTTGAGTTGT | CTTGTGAGCCA | GTTCAGGGAGTT |
| | (SEQ ID | (SEQ ID | TATCTCCTTTT |
| | NO: 1) | NO: 2) | (SEQ ID |
| | | | NO: 3) |
| GADD34 | CGGACCCTG | AAGGCCAGAAAG | GAAATGGACAG |
| | AGACTCCCC | GTGCGCTTCTC | TGACCTTCTCG |
| | (SEQ ID | (SEQ ID | (SEQ ID |
| | NO: 4) | NO: 5) | NO: 6) |
| GADD153 | GCAAGAGGTCCT | CACCTCCTGGAAA | GGGTCAAGAGTG |
| | GTCTTCAGATG | TGAAGAGGAATCA | GTGAAGATTTTT |
| | (SEQ ID | (SEQ ID | (SEQ ID |
| | NO: 7) | NO: 8) | NO: 9) |
| 18S rRNA | CGGCTACCAC | GCTGGAATTAC | TGCTGGCACCAG |
| | ATCCAAGGAA | CGCGGCT | ACTTGCCCTC |
| | (SEQ ID | (SEQ ID | (SEQ ID |
| | NO: 10) | NO: 11) | NO: 12) |

### BIBLIOGRAPHY

1. Berneburg, M.; Clingen, P. H.; Harcourt, S. A.; Lowe, J. E.; Taylor, E. M.; Green, M. H.; Krutmann, J.; Arlett, C. F.; and Lehmann, A. R., The cancer-free phenotype in trichothiodystrophy is unrelated to its repair defect., Cancer Res, 60:431-8, 2000.
2. Kligman, A. M., Early destructive effect of sunlight on human skin, JAMA, 210:2377-80, 1969.
3. IARC, eds., Solar and UV radiation, Lyon: IARC, 1992.
4. Freeman, R. G., Data on the action spectrum for ultraviolet carcinogenesis, J Natl Cancer Inst, 55:1119-22, 1975.
5. Tyrrell, R. M., Activation of mammalian gene expression by the UV component of sunlight—from models to reality, Bioessays, 18:139-48, 1996.
6. Tyrrell, R. M., UV activation of mammalian stress proteins, Exs, 77:255-71, 1996.
7. Yasui, H. and Sakurai, H., Chemiluminescent detection and imaging of reactive oxygen species in live mouse skin exposed to UVA, Biochem Biophys Res Commun, 269:131-6, 2000.
8. Black, H. S., Potential involvement of free radical reactions in ultraviolet light-mediated cutaneous damage, Photochem Photobiol, 46:213-21, 1987.
9. Dalle Carbonare, M. and Pathak, M. A., Skin photosensitizing agents and the role of reactive oxygen species in photoaging, J Photochem Photobiol B, 14:105-24, 1992.
10. Grether-Beck, S. and Krutmann, J., Gene regulation by ultraviolet A radiation and singlet oxygen, Methods Enzymol, 319:280-90, 2000.
11. Basu-Modak, S. and Tyrrell, R. M., Singlet oxygen: a primary effector in the ultraviolet A/near-visible light induction of the human heme oxygenase gene, Cancer Res, 53:4505-10, 1993.
12. Scharffetter-Kochanek, K.; Wlaschek, M.; Briviba, K.; and Sies, H., Singlet oxygen induces collagenase expression in human skin fibroblasts, FEBS Lett, 331:304-6, 1993.
13. Wlaschek, M.; Briviba, K.; Stricklin, G. P.; Sies, H. and Scharffetter-Kochanek, K., Singlet oxygen may mediate the ultraviolet A-induced synthesis of interstitial collagenase, J Invest Dermatol, 104:194-8, 1995.
14. Keyse, S. M. and Tyrrell, R. M., Heme oxygenase is the major 32-kDa stress protein induced in human skin fibroblasts by UVA radiation, hydrogen peroxide, and sodium arsenite, Proc Natl Acad Sci USA, 86:99-103, 1989.
15. Wlaschek, M.; Heinen, G.; Poswig, A.; Schwarz, A.; Krieg, T. and Scharffetter-Kochanek, K., UVA-induced autocrine stimulation of fibroblast-derived collagenase/MMP-1 by interrelated loops of interleukin-1 and interleukin-6, Photochem Photobiol, 59:550-6, 1994.
16. Grether-Beck, S.; Olaizola-Horn, S.; Schmitt, H.; Grewe, M.; Jahnke, A.; Johnson, J. P.; Briviba, K.; Sies, H. and Krutmann, J., Activation of transcription factor AP-2 mediates UVA radiation- and singlet oxygen-induced expression of the human intercellular adhesion molecule 1 gene, Proc Natl Acad Sci USA, 93:14586-91, 1996.
17. Foote, C. S., Mechanisms of photosensitized oxidation. There are several different types of photosensitized oxidation which may be important in biological systems, Science, 162:963-970, 1968.
18. Merkel, P.; Nilsson, R. and Kearns, D. R., Deuterium effects on singlet oxygen lifetime in solutions, A new test of singlet oxygen reactions, J Am Chem Soc, 94:1030-1031, 1972.
19. Truscott, T., The photophysics and photochemistry of the carotenoids, J Photochem Photobiol B:Biol, 6:359-371, 1990.
20. Conn, P. F.; Lambert, C.; Land, E. J.; Schalch, W. and Truscott, T. G., Carotene-oxygen radical interactions, Free Radic Res Commun, 16:401-8, 1992.
21. Conn, P. F.; Schalch, W. and Truscott, T. G., The singlet oxygen and carotenoid interaction, J Photochem Photobiol B, 11:41-7, 1991.
22. Vahlquist, A.; Lee, J. B.; Michaelsson, G. and Rollman, O., Vitamin A in human skin: II Concentrations of carotene, retinol and dehydroretinol in various components of normal skin, J Invest Dermatol, 79:94-7, 1982.
23. Lee, R.; Mathews-Roth, M. M.; Pathak, M. A. and Parrish, J. A., The detection of carotenoid pigments in human skin, J Invest Dermatol, 64:175-7, 1975.
24. Biesalski, H. K.; Hemmes, C.; Hopfenmuller, W.; Schmid, C. and Gollnick, H. P., Effects of controlled exposure of sunlight on plasma and skin levels of beta-carotene, Free Radic Res, 24:215-24, 1996.
25. Davison, P. A.; Hunter, C. N. and Horton, P., Overexpression of beta-carotene hydroxylase enhances stress tolerance in Arabidopsis, Nature, 418:203-6, 2002.
26. Mathews-Roth, M. M.; Pathak, M. A.; Parrish, J.; Fitzpatrick, T. B.; Kass, E. H.; Toda, K. and Clemens, W., A clinical trial of the effects of oral beta-carotene on the responses of human skin to solar radiation, J Invest Dermatol, 59:349-53, 1972.
27. Mathews-Roth, M. M.; Pathak, M. A.; Fitzpatrick, T. B.; Harber, L. H. and Kass, E. H., Beta carotene therapy for erythropoietic protoporphyria and other photosensitivity diseases, Arch Dermatol, 113:1229-32, 1977.
28. Mathews-Roth, M. M., Erythropoietic protoporphyria: treatment with antioxidants and potential cure with gene therapy, Methods Enzymol, 319:479-84, 2000.
29. Anstey, A. V.; Ryan, A.; Rhodes, L. E.; Charman, C. R.; Arlett, C. F.; Tyrrell, R. M.; Taylor, C. R. and Pearse, A. D., Characterization of photosensitivity in the Smith-Lemli-Opitz syndrome: a new congenital photosensitivity syndrome, Br J Dermatol, 141:406-14, 1999.
30. Thomas, J. P.; Hall, R. D. and Girotti, A. W., Singlet oxygen intermediacy in the photodynamic action of membrane-bound hematoporphyrin derivative, Cancer Lett, 35:295-302, 1987.
31. Heinrich, U.; Gartner, C.; Wiebusch, M.; Eichler, O.; Sies, H.; Tronnier, H. and Stahl, W., Supplementation with beta-Carotene or a Similar Amount of Mixed Carotenoids Protects Humans from UV-Induced Erythema, J Nutr, 133:98-101, 2003.
32. Stahl, W. and Sies, H., Carotenoids and protection against solar UV radiation, Skin Pharmacol Appl Skin Physiol, 15:291-6, 2002.
33. Stahl, W.; Heinrich, U.; Jungmann, H.; Sies, H. and Tronnier, H., Carotenoids and carotenoids plus vitamin E protect against ultraviolet light-induced erythema in humans, Am J Clin Nutr, 71:795-8, 2000.
34. Alaluf, S.; Heinrich, U.; Stahl, W.; Tronnier, H. and Wiseman, S., Dietary carotenoids contribute to normal human skin color and UV photosensitivity, J Nutr, 132:399-403, 2002.
35. Gollnick, H. P. M.; HopfenmUller, W.; Hemmes, C.; Chun, S. C.; Schmid, C.; Sundermeier, K. and Biesalski, H. K., Systemic n-carotene plus topical UV-sunscreen are an optimal protection against harmful effects of natural UV-sunlight: results of the Berlin-Eilath study, Eur J Dermatol, 6:200-205, 1996.
36. Lee, J.; Jiang, S.; Levine, N. and Watson, R. R., Carotenoid supplementation reduces erythema in human skin after simulated solar radiation exposure, Proc Soc Exp Biol Med, 223:170-4, 2000.
37. Bialy, T. L.; Rothe, M. J. and Grant-Kels, J. M., Dietary factors in the prevention and treatment of nonmelanoma skin cancer and melanoma, Dermatol Surg, 28:1143-52, 2002.
38. Sayre, R. M. and Black, H. S., Beta-carotene does not act as an optical filter in skin, J Photochem Photobiol B, 12:83-90, 1992.
39. Wei, R. R.; Warner, W. G.; Lambert, L. A. and Kornhauser, A., beta-Carotene uptake and effects on intracellular levels of retinol in vitro. Nutr Cancer, 30:53-8, 1998.
40. Andersson, E.; Vahlquist, A. and Rosdahl, I., Beta-carotene uptake and bioconversion to retinol differ between human melanocytes and keratinocytes, Nutr Cancer, 39:300-6, 2001.
41. Griffiths, C. E.; Kang, S.; Ellis, C. N.; Kim, K. J.; Finkel, L. J.; Ortiz-Ferrer, L. C.; White, G. M.; Hamilton, T. A. and Voorhees, J. J., Two concentrations of topical tretinoin (retinoic acid) cause similar improvement of photoaging but different degrees of irritation. A double-blind, vehicle-controlled comparison of 0.1% and 0.025% tretinoin creams, Arch Dermatol, 131:1037-44, 1995.
42. Kligman, A. M., Topical retinoic acid (tretinoin) for photoaging: conceptions and misperceptions, Cutis, 57:142-4, 1996.
43. Fisher, G. J.; Wang, Z. Q.; Datta, S. C.; Varani, J.; Kang, S. and Voorhees, J. J., Pathophysiology of premature skin aging induced by ultraviolet light, N Engl J Med, 337:1419-28, 1997.
44. Fisher, G. J.; Talwar, H. S.; Lin, J.; Lin, P.; McPhillips, F.; Wang, Z.; Li, X.; Wan, Y.; Kang, S. and Voorhees, J. J., Retinoic acid inhibits induction of c-Jun protein by ultraviolet radiation that occurs subsequent to activation of mitogen-activated protein kinase pathways in human skin in vivo, J Clin Invest, 101:1432-40, 1998.
45. Fisher, G. J.; Talwar, H. S.; Lin, J. and Voorhees, J. J., Molecular mechanisms of photoaging in human skin in vivo and their prevention by all-trans retinoic acid, Photochem Photobiol, 69:154-7, 1999.
46. Fisher, G. J.; Datta, S.; Wang, Z.; Li, X. Y.; Quan, T.; Chung, J. H.; Kang, S. and Voorhees, J. J., c-Jun-dependent inhibition of cutaneous procollagen transcription following ultraviolet irradiation is reversed by all-trans retinoic acid, J Clin Invest, 106:663-70, 2000.
47. Griffiths, C. E., Dowling Oration delivered at the Royal College of Physicians, London, Friday 5 Jun. 1998, Retinoids: renaissance and reformation, Clin Exp Dermatol, 24:329-35, 1999.
48. Kang, S.; Duell, E. A.; Fisher, G. J.; Datta, S. C.; Wang, Z. Q.; Reddy, A. P.; Tavakkol, A.; Yi, J. Y.; Griffiths, C. E.; Elder, J. T. et al., Application of retinol to human skin in vivo induces epidermal hyperplasia and cellular retinoid binding proteins characteristic of retinoic acid but without measurable retinoic acid levels or irritation, J Invest Dermatol, 105:549-56, 1995.
49. Fuchs, E., Epidermal differentiation and keratin gene expression, J Cell Sci Suppl, 17:197-208, 1993.
50. Breitkreutz, D.; Stark, H. J.; Plein, P.; Baur, M. and Fusenig, N. E., Differential modulation of epidermal keratinization in immortalized (HaCaT) and tumorigenic human skin keratinocytes (HaCaT-ras) by retinoic acid and extracellular Ca2+, Differentiation, 54:201-17, 1993.
51. Varani, J.; Zeigler, M.; Dame, M. K.; Kang, S.; Fisher, G. J.; Voorhees, J. J.; Stoll, S. W. and Elder, J. T., Heparin-binding epidermal-growth-factor-like growth factor activation of keratinocyte ErbB receptors Mediates epidermal hyperplasia, a prominent side-effect of retinoid therapy, J Invest Dermatol, 117:1335-41, 2001.
52. Frederick, J. and Alberts, A., The natural UVA radiation environment, in: Urbach, F., eds., Biological responses to UVA radiation, Kansas: Valdenmar Publishing, 1992:7-18
53. Wertz, K.; Buchwald Hunziker, P.; Seifert, N.; Riss, G.; Neeb, M.; Steiner, G.;

Hunziker, W. and Goralczyk, R., β-Carotene interferes with UVA-induced gene expression by multiple pathways, in prep., 2003.
54. Ahn, K. S.; Moon, K. Y.; Lee, J. and Kim, Y. S., Downregulation of NF-kappaB activation in human keratinocytes by melanogenic inhibitors, J Dermatol Sci, 31:193-201, 2003.
55. Fukunaga, M.; Oka, M.; Ichihashi, M.; Yamamoto, T.; Matsuzaki, H. and Kikkawa, U. UV-induced tyrosine phosphorylation of PKC delta and promotion of apoptosis in the HaCaT cell line. Biochem Biophys Res Commun 289:573-9; 2001.
56. Chen, W.; Tang, Q.; Gonzales, M. S. and Bowden, G. T., Role of p38 MAP kinases and ERK in mediating ultraviolet-B induced cyclooxygenase-2 gene expression in human keratinocytes, Oncogene, 20:3921-6, 2001.
57. Saliou, C.; Kitazawa, M.; McLaughlin, L.; Yang, J. P.; Lodge, J. K.; Tetsuka, T.; Iwasaki, K.; Cillard, J.; Okamoto, T. and Packer, L., Antioxidants modulate acute solar ultraviolet radiation-induced NF-kappa-B activation in a human keratinocyte cell line, Free Radic Biol Med, 26:174-83, 1999.
58. Assefa, Z.; Garmyn, M.; Bouillon, R.; Merlevede, W.; Vandenheede, J. R. and Agostinis, P., Differential stimulation of ERK and JNK activities by ultraviolet B irradiation and epidermal growth factor in human keratinocytes, J Invest Dermatol 108:886-91, 1997.
59. Sitailo, L. A.; Tibudan, S. S. and Denning, M. F., Activation of caspase-9 is required for UV-induced apoptosis of human keratinocytes, J Biol Chem, 277:19346-52, 2002.
60. Leccia, M. T.; Richard, M. J.; Joanny-Crisci, F. and Beani, J. C., UV-A1 cytotoxicity and antioxidant defence in keratinocytes and fibroblasts, Eur J Dermatol, 8:478-82, 1998.
61. Li, D.; Turi, T. G.; Schuck, A.; Freedberg, I. M.; Khitrov, G. and Blumenberg, M., Rays and arrays: the transcriptional program in the response of human epidermal keratinocytes to UVB illumination, Faseb J, 15:2533-5, 2001.
62. Takao, J.; Ariizumi, K.; Dougherty, I I and Cruz, P. D., Jr., Genomic scale analysis of the human keratinocyte response to broad-band ultraviolet-B irradiation, Photodermatol Photoimmunol Photomed, 18:5-13, 2002.
63. Djavaheri-Mergny, M.; Mergny, J. L.; Bertrand, F.; Santus, R.; Maziere, C.; Dubertret, L. and Maziere, J. C., Ultraviolet-A induces activation of AP-1 in cultured human keratinocytes, FEBS Lett, 384:92-6, 1996.
64. Boukamp, P.; Petrussevska, R. T.; Breitkreutz, D.; Hornung, J.; Markham, A. and Fusenig, N. E., Normal keratinization in a spontaneously immortalized aneuploid human keratinocyte cell line, J Cell Biol, 106:761-71, 1988.
65. Edstrom, D. W., Porwit, A. and Ros, A. M., Effects on human skin of repetitive ultraviolet-A1 (UVA1) irradiation and visible light, Photodermatol Photoimmunol Photomed, 17:66-70, 2001.
66. Cantrell, A.; McGarvey, D. J.; George Truscott, T.; Rancan, F. and Bohm, F., Singlet oxygen quenching by dietary carotenoids in a model membrane environment, Arch Biochem Biophys, 412:47-54, 2003.
67. Truscott, G., Reduction potential and radical reactions of carotenoids, Meeting abstract. 13th International Carotenoid Symposium, Honolulu, 2002.
68. Yin, L.; Morita, A. and Tsuji, T., Alterations of extracellular matrix induced by tobacco smoke extract, Arch Dermatol Res, 292:188-94, 2000.
69. Herrmann, G.; Wlaschek, M.; Bolsen, K.; Prenzel, K.; Goerz, G. and Scharffetter-Kochanek,
K., Photosensitization of uroporphyrin augments the ultraviolet A-induced synthesis of matrix metalloproteinases in human dermal fibroblasts, J Invest Dermatol 107:398-403, 1996.
70. Fisher, G. J.; Datta, S. C.; Talwar, H. S.; Wang, Z. Q.; Varani, J.; Kang, S. and
Voorhees, J. J., Molecular basis of sun-induced premature skin ageing and retinoid antagonism, Nature, 379:335-9, 1996.
71. Wlaschek, M.; Wenk, J.; Brenneisen, P.; Briviba, K.; Schwarz, A.; Sies, H. and
Scharffetter-Kochanek, K., Singlet oxygen is an early intermediate in cytokine-dependent ultraviolet-A induction of interstitial collagenase in human dermal fibroblasts in vitro, FEBS Lett, 413:239-42, 1997.
72. Gack, S.; Vallon, R.; Schaper, J.; Ruther, U. and Angel, P., Phenotypic alterations in fos-transgenic mice correlate with changes in Fos/Jun-dependent collagenase type I expression. Regulation of mouse metalloproteinases by carcinogens, tumor promoters, cAMP, and Fos oncoprotein, J Biol Chem, 269:10363-9, 1994.
73. Edge, R.; Land, E. J.; McGarvey, D.; Mulroy, L. and Truscott, T. G., Relative one-electron reduction potentials of carotenoid radical cations with the vitamin E radical cation, J Am Chem Soc, 120:1998.
74. Paik, J.; During, A.; Harrison, E. H.; Mendelsohn, C. L.; Lai, K. and Blaner, W. S., Expression and characterization of a murine enzyme able to cleave beta-carotene, The formation of retinoids, J Biol Chem, 276:32160-8, 2001.
75. Yan, W.; Jang, G. F.; Haeseleer, F.; Esumi, N.; Chang, J.; Kerrigan, M.; Campochiaro, M.; Campochiaro, P.; Palczewski, K. and Zack, D. J., Cloning and characterization of a human beta,beta-carotene-15,15′-dioxygenase that is highly expressed in the retinal pigment epithelium, Genomics, 72:193-202, 2001.
76. von Lintig, J. and Wyss, A., Molecular analysis of vitamin A formation: cloning and characterization of beta-carotene 15,15′-dioxygenases, Arch Biochem Biophys, 385:47-52, 2001.
77. Wyss, A.; Wirtz, G.; Woggon, W.; Brugger, R.; Wyss, M.; Friedlein, A.; Bachmann, H. and Hunziker, W., Cloning and expression of beta,beta-carotene 15,15′-dioxygenase, Biochem Biophys Res Commun, 271:334-6, 2000.
78. Kiefer, C.; Hessel, S.; Lampert, J. M.; Vogt, K.; Lederer, M. O.; Breithaupt, D. E. and von Lintig, J., Identification and characterization of a mammalian enzyme catalyzing the asymmetric oxidative cleavage of provitamin, A. J Biol Chem, 276:14110-6, 2001.
79. Trekli, M. C.; Riss, G.; Goralczyk, R. and Tyrrell, R. M., Beta-carotene suppresses UVA-induced HO-1 gene expression in cultured FEK4, Free Radic Biol Med, 34:456-464, 2003.
80. Sorg, O.; Tran, C.; Carraux, P.; Didierjean, L.; Faison, F. and Saurat, J. H., Oxidative stress-independent depletion of epidermal vitamin A by UVA, J Invest Dermatol, 118:513-8, 2002.
81. Roe, D. A., Photodegradation of carotenoids in human subjects, Fed Proc, 46:1886-9, 1987.
82. Brenneisen, P.; Sies, H. and Scharffetter-Kochanek, K., Ultraviolet-B irradiation and matrix metalloproteinases: from induction via signaling to initial events, Ann N Y Acad Sci, 973:31-43, 2002.
83. Kick, G.; Messer, G.; Plewig, G.; Kind, P. and Goetz, A. E., Strong and prolonged induction of c-jun and c-fos proto-oncogenes by photodynamic therapy, Br J Cancer, 74:30-6, 1996.
84. Gange, R. W., Comparison of pigment responses in human skin to UVB and UVA radiation, Prog Clin Biol Res, 256:475-85, 1988.
85. Ariizumi, K.; Bergstresser, P. R. and Takashima, A., Wavelength-specific induction of immediate early genes by ultraviolet radiation, J Dermatol Sci, 12:147-55, 1996.
86. Obermuller-Jevic, U. C.; Francz, P. I.; Frank, J.; Flaccus, A. and Biesalski, H. K., Enhancement of the UVA induction of haem oxygenase-1 expression by beta-carotene in human skin fibroblasts, FEBS Lett, 460:212-6, 1999.
87. Obermuller-Jevic, U. C.; Schlegel, B.; Flaccus, A. and Biesalski, H. K., The effect of beta-carotene on the expression of interleukin-6 and heme oxygenase-1 in UV-irradiated human skin fibroblasts in vitro., FEBS Lett, 509:186-90, 2001.
88. Offord, E. A.; Gautier, J. C.; Avanti, O.; Scaletta, C.; Runge, F.; Kramer, K. and
Applegate, L. A., Photoprotective potential of lycopene, beta-carotene, vitamin E, vitamin C and carnosic acid in UVA-irradiated human skin fibroblasts, Free Radic Biol Med, 32:1293-303, 2002.
89. Moran, M. and Miceli, M. C., Engagement of GPI-linked CD48 contributes to TCR signals and cytoskeletal reorganization: a role for lipid rafts in T cell activation, Immunity, 9:787-96, 1998.
90. Roy, S.; Luetterforst, R.; Harding, A.; Apolloni, A.; Etheridge, M.; Stang, E.; Rolls, B.; Hancock, J. F. and Parton, R. G., Dominant-negative caveolin inhibits H-Ras function by disrupting cholesterol-rich plasma membrane domains, Nat Cell Biol, 1:98-105, 1999.
91. Eicker, J.; Kurten, V.; Wild, S.; Riss, G.; Goralczyk, R.; Krutmann, J. and Berneburg, M., Betacarotene supplementation protects from photoaging-associated mitochondrial DNA mutation, Photochem Photobiol Sci, 2:655-9, 2003.
92. Eichler, O.; Sies, H. and Stahl, W., Divergent optimum levels of lycopene, beta-carotene and lutein protecting against UVB irradiation in human fibroblastst, Photochem Photobiol, 75:503-6, 2002.
93. Kahari, V. M. and Saarialho-Kere, U., Matrix metalloproteinases in skin, Exp Dermatol, 6:199-213, 1997.
94. Sternlicht, M. D. and Werb, Z., How matrix metalloproteinases regulate cell behavior, Annu Rev Cell Dev Biol, 17:463-516, 2001.
95. Brennan, M.; Bhatti, H.; Nerusu, K. C.; Bhagavathula, N.; Kang, S.; Fisher, G. J.; Varani, J. and Voorhees, J. J., Matrix metalloproteinase-1 is the major collagenolytic enzyme responsible for collagen damage in UV-irradiated human skin, Photochem Photobiol, 78:43-8, 2003.
96. Berneburg, M.; Grether-Beck, S.; Kurten, V.; Ruzicka, T.; Briviba, K.; Sies, H. and Krutmann, J., Singlet oxygen mediates the UVA-induced generation of the photoaging-associated mitochondrial common deletion, J Biol Chem, 274:15345-9, 1999.
97. Valacchi, G.; Rimbach, G.; Saliou, C.; Weber, S. U. and Packer, L., Effect of benzoyl peroxide on antioxidant status, NF-kappaB activity and interleukin-lalpha gene expression in human keratinocytes, Toxicology, 165:225-34, 2001.
98. Shindo, Y.; Witt, E. and Packer, L., Antioxidant defense mechanisms in murine epidermis and dermis and their responses to ultraviolet light, J Invest Dermatol, 100:260-5, 1993.
99. Shindo, Y.; Witt, E.; Han, D. and Packer, L., Dose-response effects of acute ultraviolet irradiation on antioxidants and molecular markers of oxidation in murine epidermis and dermis, J Invest Dermatol, 102:470-5, 1994.
100. Torma, H.; Rollman, 0. and Vahlquist, A., The vitamin A metabolism and expression of retinoid-binding proteins differ in HaCaT cells and normal human keratinocytes, Arch Dermatol Res, 291:339-45, 1999.
101. Popa, C.; Dicker, A. J.; Dahler, A. L. and Saunders, N. A., Cytochrome P450, CYP26AI, is expressed at low levels in human epidermal keratinocytes and is not retinoic acid-inducible, Br J Dermatol, 141:460-8, 1999.
102. Wang, X. D.; Russell, R. M.; Liu, C.; Stickel, F.; Smith, D. E. and Krinsky, N. I., Beta-oxidation in rabbit liver in vitro and in the perfused ferret liver contributes to retinoic acid biosynthesis from beta-apocarotenoic acids, J Biol Chem, 271:26490-8, 1996.
103. Bachmann, H.; Desbarats, A.; Pattison, P.; Sedgewick, M.; Riss, G.; Wyss, A.; Cardinault, N.; Duszka, C.; Goralczyk, R. and Grolier, P., Feedback regulation of beta,beta-carotene 15,15′-monooxygenase by retinoic acid in rats and chickens, J Nutr, 132:3616-22, 2002.
104. Boulanger, A.; McLemore, P.; Copeland, N. G.; Gilbert, D. J.; Jenkins, N. A.; Yu, S. S.; Gentleman, S. and Redmond, T. M., Identification of beta-carotene 15, 15′-monooxygenase as a peroxisome proliferator-activated receptor target gene, Faseb J, 17:1304-6, 2003.
105. Takeda, A.; Morinobu, T.; Takitani, K.; Kimura, M. and Tamai, H., Measurement of retinoids and beta-carotene 15,15′-dioxygenase activity in HR-1 hairless mouse skin with UV exposure, J Nutr Sci Vitaminol (Tokyo), 49:69-72, 2003.
106. Elder, J. T.; Fisher, G. J.; Zhang, Q. Y.; Eisen, D.; Krust, A.; Kastner, P.; Chambon, P. and Voorhees, J. J., Retinoic acid receptor gene expression in human skin, J Invest Dermatol, 96:425-33, 1991.
107. Wang, Z.; Boudjelal, M.; Kang, S.; Voorhees, J. J. and Fisher, G. J., Ultraviolet irradiation of human skin causes functional vitamin A deficiency, preventable by all-trans retinoic acid pre-treatment, Nat Med, 5:418-22, 1999.
108. Hoffmann, B.; Lehmann, J. M.; Zhang, X. K.; Hermann, T.; Husmann, M.; Graupner, G. and Pfahl, M., A retinoic acid receptor-specific element controls the retinoic acid receptor-beta promoter, Mol Endocrinol, 4:1727-36, 1990.
109. Sucov, H. M.; Murakami, K. K. and Evans, R. M., Characterization of an autoregulated response element in the mouse retinoic acid receptor type beta gene, Proc Natl Acad Sci USA, 87:5392-6, 1990.
110. Brand, N. J.; Petkovich, M. and Chambon, P., Characterization of a functional promoter for the human retinoic acid receptor-alpha (hRAR-alpha), Nucleic Acids Res, 18:6799-806, 1990.
111. Lehmann, J. M.; Zhang, X. K. and Pfahl, M., RAR gamma 2 expression is regulated through a retinoic acid response element embedded in Sp1 sites, Mol Cell Blol, 12:2976-85, 1992.
112. Barger, P. M. and Kelly, D. P., Identification of a retinoid/chicken ovalbumin upstream promoter transcription factor response element in the human retinoid X receptor gamma2 gene promoter, J Biol Chem, 272:2722-8, 1997.
113. Guerin, E.; Ludwig, M. G.; Basset, P. and Anglard, P., Stromelysin-3 induction and interstitial collagenase repression by retinoic acid. Therapeutical implication of receptor-selective retinoids dissociating transactivation and AP-1-mediated transrepression, J Biol Chem, 272:11088-95, 1997.
114. Pan, L.; Eckhoff, C. and Brinckerhoff, C. E., Suppression of collagenase gene expression by all-trans and 9-cis retinoic acid is ligand dependent and requires both RARs and RXRs, J Cell Biochem, 57:575-89, 1995.
115. Lehmann, B., HaCaT cell line as a model system for vitamin D3 metabolism in human skin, J Invest Dermatol, 108:78-82, 1997.
116. Tetlow, L. C. and Woolley, D. E., The effects of 1 alpha, 25-dihydroxyvitamin D(3) on matrix metalloproteinase and prostaglandin E(2) production by cells of the rheumatoid lesion, Arthritis Res, 1:63-70, 1999.
117. Balmer, J. E. and Blomhoff, R, Gene expression regulation by retinoic acid, J Lipid Res, 43:1773-808, 2002.
118. Chaturvedi, V.; Qin J. Z.; Denning M. F.; Choubey D.; Diaz M. O. and Nickoloff B. J., Abnormal NF-kappaB signaling pathway with enhanced susceptibility to apoptosis in immortalized keratinocytes, J Dermatol Sci, 26:67-78, 2001.
119. Edge, R.; Land, E. J.; McGarvey, D. J.; Burke, M. and Truscott T. G., The reduction potential of the beta-carotene.+/beta-carotene couple in an aqueous micro-heterogeneous environment, FEBS Lett, 471:125-7, 2000.
120. Fuller, C. J.; Faulkner, H; Bendich, A.; Parker, R. S. and Roe, D. A., Effect of beta-carotene supplementation on photosuppression of delayed-type hypersensitivity in normal young men, Am J Clin Nutr, 56:684-90, 1992.
121. Garmyn, M.; Ribaya-Mercado, J. D.; Russel, R. M.; Bhawan, J. and Gilchrest, B. A., Effect of beta-carotene supplementation on the human sunburn reaction, Exp Dermatol, 4:104-11, 1995.
122. Gille, J.; Reisinger, K.; Asbe-Vollkopf, A.; Hardt-Weinelt, K. and Kaufmann, R., Ultraviolet-A-induced transactivation of the vascular endothelial growth factor gene in HaCaT keratinocytes is conveyed by activator protein-2 transcription factor, J Invest Dermatol, 115:30-6, 2000.
123. Gollnick, H. P. M.; HopfenmUller, W.; Hemmes, C.; Chun S C.; Schmid, C.; Sundermeier, K. and Biesalski, H. K., Systemic β-carotene plus topical UV-sunscreen are an optimal protection against harmful effects of natural UV-sunlight: results of the Berlin-Eilath study, Eur J Dermatol, 6:200-205, 1996.
124. Green, A.; Williams, G.; Neale, R. et al., Daily sunscreen application and betacarotene supplementation in prevention of basal-cell and squamous-cell carcinomas of the skin: a randomised controlled trial, Lancet, 354:723-9, 1999.
125. Greenberg, E. R.; Baron, J. A.; Stukel, T. A, et al., A clinical trial of beta carotene to prevent basal-cell and squamous-cell cancers of the skin, The Skin Cancer Prevention Study Group, N Engl J Med, 323:789-95, 1990.
126. He, Y. Y.; Huang, J. L.; Sik, R. H.; Liu J.; Waalkes M. P. and Chignell, C. F., Expression profiling of human keratinocyte response to ultraviolet A: implications in apoptosis, J Invest Dermatol, 122:533-43, 2004.
127. Hughes, D. A., Dietary carotenoids and human immune function, Nutrition, 17:823-7, 2001.
128. Krinsky, N. I. and Yeum K. J., Carotenoid-radical interactions, Biochem Biophys Res Commun, 305:754-60, 2003.
129. Mathews-Roth, M. M., Antitumor activity of beta-carotene, canthaxanthin and phytoene, Oncology, 39:33-7, 1982.
130. Postaire E, Jungmann H, Bejot M, Heinrich U, Tronnier H: Evidence for antioxidant nutrients-induced pigmentation in skin: results of a clinical trial. Biochem Mol Biol Int 42:1023-33, 1997
131. Raap W P, Tronnier H, Wiskemann A: Photoprotection and skin coloring by oral carotenoids. Dermatologica 171:371-3, 1985
132. Siler U, Barella L, Spitzer V, Schnorr J, Lein M, Goralczyk R, Wertz K: Lycopene and vitamin E interfere with autocrine/paracrine loops in the Dunning prostate cancer model. Faseb J 18:1019-21, 2004
133. Sorg, O.; Tran, C.; Carraux, P.; Didierjean, L.; Faison, F. and Saurat, J. H., Oxidative stress-independent depletion of epidermal vitamin A by UVA, J Invest Dermatol, 118:513-8, 2002.
134. Stahl, W.; Heinrich, U.; Jungmann, H.; Sies, H. and Tronnier, H., Carotenoids and carotenoids plus vitamin E protect against ultraviolet light-induced erythema in humans, Am J Clin Nutr, 71:795-8, 2000.
135. Thurmann, P. A.; Steffen, J.; Zwernemann, C.; Aebischer, C. P.; Cohn, W.; Wendt,
G. and Schalch, W., Plasma concentration response to drinks containing beta-carotene as carrot juice or formulated as a water dispersible powder, Eur J Nutr, 41:228-35, 2002.
136. Tronnier, H., Protective effect of beta-carotene and canthaxanthin against UV reactions of the skin, Z Haufkr, 59:859-70, 1984.
137. Wertz, K.; Seifert, N.; Buchwald Hunziker, P.; Riss, G.; Wyss, A.; Lankin, C. and Goralczyk, R., β-Carotene inhibits UVA-induced MMP 1 and 10 expression in keratinocytes by a singlet oxygen-dependent mechanism, Free Radic Biol Med, 37:654-670, 2004.

The scope of the present invention is not limited by the description, examples, and suggested uses herein, and modifications may be made without departing from the spirit of the invention. Thus, it is intended that the present invention cover modifications and variations of this invention provided that they come within the scope of the appended Claims and their equivalents.

1. A method for screening for a compound for the treatment and/or prevention of skin aging, comprising:
a) contacting a sample of eukaryotic cells with the compound to be evaluated to produce pretreated cells;
b) irradiating the pretreated cells, and control cells that were not contacted with the compound, with UV radiation;
c) quantifying the expression level of at least one MMP gene in the pretreated cells and in the control cells,
d) comparing the expression level of the at least one MMP gene in the pretreated cells to the expression level of the at least one MMP gene in the control cells; and
e) identifying compounds that inhibit UVA-induction of said at least one MMP gene as indicated by significantly reduced UVA-induced MMP expression in the treated cells as compared to the control cells.

2. The method of claim 1, wherein the eukaryotic cells are keratinocytes.

3. The method of claim 1, wherein the at least one MMP gene is MMP-1.

4. The method of claim 1, wherein the at least one MMP gene is MMP-3.

5. The method of claim 1, wherein the at least one MMP gene is MMP-10.

6. The method of claim 1, wherein the expression level of the at least one MMP gene is quantified at the RNA level by PCR, Northern blotting and/or microarray.

7. The method of claim 1, wherein the expression level of the at least one MMP gene is quantified at the protein level by ELISA, Western blotting and/or microarray.

8. The method of claim 1, wherein the compound inhibits UVA-induction of the at least one MMP gene by at least 30%.

9. The method of claim 1, wherein the expression level of MMP-1, MMP2 and MMP-3 is quantified by microarray.

10. The method of claim 9, wherein the microarray includes at least one control selected from the group consisting of normalization controls, mismatch controls and expression level controls.

11. The method of claim 10 wherein the control is a housekeeper gene.

12. The method of claim 9, wherein the microarray further includes genes selected from the group consisting of: immediate early genes, oxidative defense genes, extracellular matrix genes, pro-inflammatory genes, VEGF-related ligand and receptor genes, IFN alpha/beta genes, interleukin genes, proteinase-activated receptor genes, prostaglandin synthesis and signalling genes, EGF-related ligand and receptor genes, FGF-related ligand and receptor genes, TGF-beta-related ligand and receptor genes, Wnt signalling genes, IGF/insulin signalling genes, Jagged/Delta signalling genes, MAPK pathway genes, differentiation marker genes, cell cycle genes, apoptosis genes, and combinations thereof.

13. The method of claim 9, wherein the microarray is a nucleotide microarray.

14. The method of claim 13, wherein the nucleotide microarray is a cDNA microarray.

15. The method of claim 13, where the nucleotide microarray is an oligonucleotide microarray.

16. The method of claim 1, wherein the wherein the expression level of MMP-1, MMP2 and MMP-3 is quantified by microarray and the quantifying step comprises calculating the difference in hybridization signal intensity between at least one nucleotide probe and its corresponding mismatch control probe.

17. The method of claim 16, further comprising calculating the average difference in hybridization signal intensity between at least one nucleotide probe and its corresponding mismatch control probe.

18. The method of claim 1, wherein the irradiating step involves doses of UVA ranging from 50 to 300 kJ/m².

19. The method of claim 18, wherein the irradiating step involves doses of UVA ranging from 270 to 300 kJ/m².

20. The method of claim 1, wherein the quantifying step is performed 5 hours after the irradiating step.

21. The method of claim 9, wherein the microarray further includes at least one gene selected from the group consisting of: C-FOS, FRA-1, JUN-D, JUN-B, MAF-F, C-MYC, OSR-1, GEM, DKK-1, GADD34, GADD153, IEX-1, TSSC3/IPL, TDAG51, serpinB1, lekti, PAR-2, VEGF, IL-6, HB-EGF, SMADs, EGFR, HER3, Wnt5A, FGFR2, cyclin E, ODC, ID1-3, ID-4, RB, KI67, thymidylate synthase, DNA ligase III, CENP-E, centromere and spindle protein genes, COL4, COL7, Cx31, BPAG1, integrin alpha6, KLF4, and ILK.
